(19) United States
(12) Patent Application Publication
(10) Pub. No.: US 2014/0275257 A1
(43) Pub. Date: Sep. 18, 2014

(54) N-ACETYL CYSTEINE COMPOSITIONS IN THE TREATMENT OF SYSTEMIC LUPUS ERYTHEMATOSUS

(71) Applicant: Foundation for the State University of New York, Albany, NY (US)

(72) Inventor: Andras Perl, Jamesville, NY (US)

(21) Appl. No.: 13/826,183

(22) Filed: Mar. 14, 2013

(51) Int. Cl. A61K 31/198; A61K 45/06

(52) U.S. Cl. CPC A61K 31/198; A61K 45/06
USPC 514/562

(57) ABSTRACT

The described invention provides a method and kit for treating a lupus condition with N-acetyl-L-cysteine (NAC) compositions that improve lupus disease activity driven by a decrease in the activity of the mammalian target of rapamycin (mTOR). The compositions of the described invention is effective to: (1) reduce fatigue; (2) reduce cognitive/inattentive component of attention deficit and hyperactivity (ADHD) self-report scale (ASRS); (3) reduce inflammation, for example, as measured by the systemic lupus erythematosus disease activity index (SLEDAI), and the British Isles Lupus Assessment Group (BILAG) score; (4) modulate mitochondrial potential and (5) reduce T cell cycle dysfunction driven by a decrease in the activity of the mammalian target of rapamycin (mTOR) in patients suffering from systemic lupus erythematosus (SLE).

### STATEMENT OF GOVERNMENT FUNDING

This invention was made with government support awarded by the National Institute of Health. The government has certain rights in the invention.

### FIELD OF THE INVENTION

The described invention relates to autoimmune disease mechanisms, oxidative stress, and regulation of T-cell differentiation and apoptosis.

### BACKGROUND

### Autoimmune Disorders

One of the most important features of the immune system is its ability to discriminate between antigenic determinants expressed on foreign substances, such as pathogenic microbes, and antigenic determinants expressed by host tissues (i.e., self-antigens). This ability of the system to ignore host antigens is an active process involving the elimination or inactivation of cells that could recognize self-antigens through a process known as immunologic tolerance.

Failures in establishing immunologic tolerance or unusual presentations of self-antigens can give rise to tissue-damaging immune responses directed against antigenic determinants on host molecules. These result in auto-immune disorders. The term “autoimmune disorder” as used herein refers to a disease, disorder or condition in which the body's immune system, which normally fights infections and viruses, is misdirected and attacks the body's own normal, healthy tissue. Examples of autoimmune disorders include, without limitation, systemic lupus erythematosus (SLE), rheumatoid arthritis, insulin-dependent diabetes mellitus, multiple sclerosis, myasthenia gravis, and regionic enteritis.

Systemic autoimmunity encompasses autoimmune conditions in which autoreactivity is not limited to a single organ or organ system. This definition includes, but is not limited to, such autoimmune diseases as systemic lupus erythematosus (SLE), systemic sclerosis (scleroderma), rheumatoid arthritis (RA), chronic graft-versus-host disease (GVHD), and various forms of vasculitis. (Paul, W. E., “Chapter 1: The immune system: an introduction,” Fundamental Immunology, 4^{th} Edition, Ed. Paul, W. E., Lippicott-Raven Publishers, Philadelphia (1999)).

Autoimmunity is caused by a complex interaction of multiple gene products, unlike immunodeficiency diseases, where a single dominant genetic trait is often the main disease determinant. (Reviewed in Fathman, C. G. et al., “An array of possibilities for the study of autoimmunity” Nature, 435(7042): 605-611 (2005); Anaya, J.-M., “Common mechanisms of autoimmune diseases (the autoimmune tautology),” Autoimmunity Reviews, 11(11): 781-784 (2012)).

Autoimmune diseases are major causes of morbidity and mortality throughout the world and are difficult to treat. (Reviewed in for example in Hayter, S. M. et al., “Updated assessment of the prevalence, spectrum and case definition of autoimmune disease,” Autoimmunity Reviews, 11(10): 754-765 (2012); and Rioux, J. D. et al., “Paths to understanding the genetic basis of autoimmune disease,” Nature, 435(7042): 584-589 (2005)).

Regulatory T (T_{Reg}) cells have been targeted for therapeutic intervention in a wide varierty of autoimmune disorders (Reviewed in Kronenberg, M. et al., “Regulation of immunity by self-reactive T cells,” Nature, 435(7042): 598-604 (2005)).

Other components of the pathological cascade in autoimmune disorders that have received attention include, for example, factors involved in lymphocyte homing to target tissues; enzymes that are critical for the penetration of blood vessels and the extracellular matrix by immune cells; cytokines that mediate pathology within the tissues; various cell types that mediate damage at the site of disease, cell antigens; specific adaptive receptors, including the T-cell receptor (TCR) and immunoglobulin; and toxic mediators, such as complement components and nitric oxide. (Reviewed in Feldmann, M. et al., “Design of effective immunotherapy for human autoimmunity,” Nature, 435(7042): 612-619 (2005)).

Although mutations in a single gene can cause autoimmunity, most autoimmune diseases are associated with multiple sequence variants. (Reviewed in Rioux, J. D. et al., “Paths to understanding the genetic basis of autoimmune disease,” Nature, 435(7042): 584-589 (2005); and Goodnow, C. C. et al., “Cellular and genetic mechanisms of self-tolerance and autoimmunity,” Nature, 435(7042): 590-596 (2005)).

Autoantibodies originate in apoptotic cells. Specifically, apoptotic vesicles exposed on the surface of apoptotic cells contain cellular debris, including nucleic acids and nucleotides. Under normal conditions, the monocytic-macrophagic system removes the apoptotic debris from circulation. The complement system, and other molecules, secreted by the apoptotic cells, also participate in this process, such as lyophosphatidyl choline that attract phagocytes as well as molecules exposed on their surface, such as oxidized phosphatidyl serine, recognized by scavenger receptors on the surface of the phagocyte such as CD36 and oxLDL, facilitating their internalization.

Systemic lupus erythematosus (SLE) is associated with enhanced apoptosis and defective clearance of apoptotic cells resulting in the occurrence of large quantities of autoantbodies. Both alterations lead to accumulation of secondary necrotic material, which may trigger inflammation, and modified nuclear fragments that act as danger signals for the immune system leading to the production of antibodies for their neutralization by self-reactive B-lymphocytes. (Reviewed in Sifuentes, W. A. et al., “New therapeutic targets in systemic lupus,” Reumatol. Clin., 8(4): 201-207 (2012)).

### The Immune System

Multicellular organisms have developed two defense mechanisms to fight infection by pathogens: innate and adaptive immune responses Innate immune responses are triggered immediately after infection and are independent of the host's prior exposure to the pathogen. Adaptive immune responses operate later in an infection and are highly specific for the pathogen that triggered them. The function of adaptive immune responses is to destroy the invading pathogens and any toxic molecules they produce. (“Chapter 24: The adaptive immune system,” Molecular Biology of the Cell, Alberts, B. et al., Garland Science, N.Y., 2002).

The immune system consists of a wide range of distinct cell types, amongst which white blood cells called lymphocytes play a central role in dertermining immune specificity. Other cells, such as monocytes, macrophages, dendritic cells, Langerhans' cells, natural killer (NK) cells, mast cells, basophils, and other members of the myeloid lineage of cells, interact with the lymphocytes and play critical functions in antigen presentation and mediation of immunologic functions. (Paul, W. E., “Chapter 1: The immune system: an introduction,” Fundamental Immunology, 4^{th} Edition, Ed. Paul, W. E., Lippicott-Raven Publishers, Philadelphia (1999)).

Lymphocytes are found in central lymphoid organs, the thymus, and bone marrow, where they undergo developmental steps that enable them to orchestrate immune responses. A large portion of lymphocytes and macrophages comprise a recirculating pool of cells found in the blood and lymph, providing the means to deliver immunocompetent cells to localized sites in need. (Paul, W. E., “Chapter 1: The immune system: an introduction,” Fundamental Immunology, 4^{th} Edition, Ed. Paul, W. E., Lippicott-Raven Publishers, Philadelphia (1999)).

Lymphocytes are specialized cells, committed to respond to a limited set of structurally related antigens. This commitment, which exists before the first contact of the immune system with a given antigen, is expressed by the presence on the lymphocyte's surface of receptors that are specific for specific determinants or epitopes on the antigen. Each lymphocyte possesses a population of cell-surface receptors, all of which have identical combining regions. One set of lymphocyte, referenced to as a “clone” differs from another in the structure of the combining region of its receptors, and thus differs in the epitopes being recognized. The ability of an organism to respond to any nonself antigen is achieved by large number of different clones of lymphocytes, each bearing receptors specific for a distinct epitope. (Paul, W. E., “Chapter 1: The immune system: an introduction,” Fundamental Immunology, 4^{th} Edition, Ed. Paul, W. E., Lippicott-Raven Publishers, Philadelphia (1999)).

There are two broad classes of adaptive immune responses that are carried out by different classes of lymphocytes: antibody responses mediated by B-lymphocytes (or B-cells); and cell-mediated immune responses carried out by T-lymphocytes (or T-cells). B-cells are bone-marrow-derived and are precursors of immunoglobulin- (Ig-) or antibody-expressing cells while T-cells are thymus-derived. (Paul, W. E., “Chapter 1: The immune system: an introduction,” Fundamental Immunology, 4^{th} Edition, Ed. Paul, W. E., Lippicott-Raven Publishers, Philadelphia (1999)).

Primary immune responses are initiated by the encounter of an individual with a foreign antigenic substance, generally an infectious microorganism. The infected individual responds with the production of immunoglobulin (Ig) molecules specific for the antigenic determinants of the immunogen and with the expansion and differentiation of antigen-specific regulatory and effector T-lymphocytes. The latter include both T-cells that secrete cytokines as well as natural killer T-cells that are capable of lysing the cell. (Paul, W. E., “Chapter 1: The immune system: an introduction,” Fundamental Immunology, 4^{th} Edition, Ed. Paul, W. E., Lippicott-Raven Publishers, Philadelphia (1999)).

As a consequence of the initial response, the immunized individual develops a state of immunologic memory. If the same (or closely related) microorganism or foreign object is encountered again, a secondary response is triggered. This generally consists of an antibody response that is more rapid and greater in magnitude than the primary (initial) response and is more effective in clearing the microbe from the body. A similar and more effective T-cell response then follows. The initial response often creates a state of immunity such that the individual is protected against a second infection, which forms the basis for immunizations. (Paul, W. E., “Chapter 1: The immune system: an introduction,” Fundamental Immunology, 4^{th} Edition, Ed. Paul, W. E., Lippicott-Raven Publishers, Philadelphia (1999)).

The immune response is highly specific. Primary immunization with a given microorganism evokes antibodies and T-cells that are specific for the antigenic determinants or epitopes found on that microorganism but that usually fail to recognize (or recognize only poorly) antigenic determinants of unrelated microbes. (Paul, W. E., “Chapter 1: The immune system: an introduction,” Fundamental Immunology, 4^{th} Edition, Ed. Paul, W. E., Lippicott-Raven Publishers, Philadelphia (1999)).

B-Lymphocytes

B-lymphocytes are derived from hematopoietic cells of the bone marrow. A mature B-cell can be activated with an antigen that expresses epitopes that are recognized by its cell surface. The activation process may be direct, dependent on cross-linkage of membrane Ig molecules by the antigen (cross-linkage-dependent B-cell activation), or indirect, via interaction with a helper T-cell, in a process referred to as cognate help. In many physiological situations, receptor cross-linkage stimuli and cognate help synergize to yield more vigorous B-cell responses. (Paul, W. E., “Chapter 1: The immune system: an introduction,” Fundamental Immunology, 4^{th} Edition, Ed. Paul, W. E., Lippicott-Raven Publishers, Philadelphia (1999)).

Cross-linkage dependent B-cell activation requires that the antigen express multiple copies of the epitope complementary to the binding site of the cell surface receptors because each B-cell expresses Ig molecules with identical variable regions. Such a requirement is fulfilled by other antigens with repetitive epitopes, such as capsular polysaccharides of microorganisms or viral envelope proteins. Cross-linkage-dependent B-cell activation is a major protective immune response mounted against these microbes. (Paul, W. E., “Chapter 1: The immune system: an introduction,” Fundamental Immunology, 4^{th} Edition, Ed. Paul, W. E., Lippicott-Raven Publishers, Philadelphia (1999)).

Cognate help allows B-cells to mount responses against antigens that cannot cross-link receptors and, at the same time, provides costimulatory signals that rescue B cells from inactivation when they are stimulated by weak cross-linkage events. Cognate help is dependent on the binding of antigen by the B-cell's membrane immunoglobulin (Ig), the endocytosis of the antigen, and its fragmentation into peptides within the endosomal/lysosomal compartment of the cell. Some of the resultant peptides are loaded into a groove in a specialized set of cell surface proteins known as class II major histocompatibility complex (MHC) molecules. The resultant class II/peptide complexes are expressed on the cell surface and act as ligands for the antigen-specific receptors of a set of T-cells designated as CD4+ T-cells. The CD4+ T-cells bear receptors on their surface specific for the B-cell's class II/peptide complex. B-cell activation depends not only on the binding of the T cell through its T cell receptor (TCR), but this interaction also allows an activation ligand on the T-cell (CD40 ligand) to bind to its receptor on the B-cell (CD40) signaling B-cell activation. In addition, T helper cells secrete several cytokines that regulate the growth and differentiation of the stimulated B-cell by binding to cytokine receptors on the B cell. (Paul, W. E., “Chapter 1: The immune system: an introduction,” Fundamental Immunology, 4^{th} Edition, Ed. Paul, W. E., Lippicott-Raven Publishers, Philadelphia (1999)).

During cognate help for antibody production, the CD40 ligand is transiently expressed on activated CD4+ T helper cells, and it binds to CD40 on the antigen-specific B cells, thereby tranducing a second costimulatory signal. The latter signal is essential for B cell growth and differentiation and for the generation of memory B cells by preventing apoptosis of germinal center B cells that have encountered antigen. Hyperexpression of the CD40 ligand in both B and T cells is implicated in the pathogenic autoantibody production in human SLE patients. (Desai-Mehta, A. et al., “Hyperexpression of CD40 ligand by B and T cells in human lupus and its role in pathogenic autoantibody production,” J. Clin. Invest., 97(9): 2063-2073 (1996)).

T-Lymphocytes

T-lymphocytes derive from precursors in hematopoietic tissue, undergo differentiation in the thymus, and are then seeded to peripheral lymphoid tissue and to the recirculating pool of lymphocytes. T-lymphocytes or T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on their expression of specific cell surface molecules and the secretion of cytokines (Paul, W. E., “Chapter 1: The immune system: an introduction,” Fundamental Immunology, 4^{th} Edition, Ed. Paul, W. E., Lippicott-Raven Publishers, Philadelphia (1999)).

T cells differ from B cells in their mechanism of antigen recognition. Immunoglobulin, the B cell's receptor, binds to individual epitopes on soluble molecules or on particulate surfaces. B-cell receptors see epitopes expressed on the surface of native molecules. Antibody and B-cell receptors evolved to bind to and to protect against microorganisms in extracellular fluids. In contrast, T cells recognize antigens on the surface of other cells and mediate their functions by interacting with, and altering, the behavior of these antigen-presenting cells (APCs). There are three main types of antigen-presenting cells in peripheral lymphoid organs that can activate T cells: dendritic cells, macrophages and B cells. The most potent of these are the dendritic cells, whose only function is to present foreign antigens to T cells. Immature dendritic cells are located in tissues throughout the body, including the skin, gut, and respiratory tract. When they encounter invading microbes at these sites, they endocytose the pathogens and their products, and carry them via the lymph to local lymph nodes or gut associated lymphoid organs. The encounter with a pathogen induces the dendritic cell to mature from an antigen-capturing cell to an antigen-presenting cell that can activate T cells. APCs display three types of protein molecules on their surface that have a role in activating a T cell to become an effector cell: (1) MHC proteins, which present foreign antigen to the T cell receptor; (2) costimulatory proteins which bind to complementary receptors on the T cell surface; and (3) cell-cell adhesion molecules, which enable a T cell to bind to the antigen-presenting cell for long enough to become activated. (“Chapter 24: The adaptive immune system,” Molecular Biology of the Cell, Alberts, B. et al., Garland Science, N.Y., 2002).

T-cells are subdivided into two distinct classes based on the cell surface receptors they express. The majority of T cells express T cell receptors (TCR) consisting of α and β chains. A small group of T cells express receptors made of γ and δ chains. Among the α/β T cells are two important sublineages: those that express the coreceptor molecule CD4 (CD4+ T cells); and those that express CD8 (CD8+ T cells). These cells differ in how they recognize antigen and in their effector and regulatory functions.

CD4+ T cells are the major regulatory cells of the immune system. Their regulatory function depends both on the expression of their cell-surface molecules, such as CD40 ligand whose expression is induced when the T cells are activated, and the wide array of cytokines they secrete when activated.

T cells also mediate important effector functions, some of which are determined by the patterns of cytokines they secrete. The cytokines can be directly toxic to target cells and can mobilize potent inflammatory mechanisms. In addition, T cells particularly CD8+ T cells, can develop into cytotoxic T-lymphocytes (CTLs) capable of efficiently lysing target cells that express antigens recognized by the CTLs. (Paul, W. E., “Chapter 1: The immune system: an introduction,” Fundamental Immunology, 4^{th} Edition, Ed. Paul, W. E., Lippicott-Raven Publishers, Philadelphia (1999)).

T cell receptors (TCRs) recognize a complex consisting of a peptide derived by proteolysis of the antigen bound to a specialized groove of a class II or class I MHC protein. The CD4+ T cells recognize only peptide/class II complexes while the CD8+ T cells recognize peptide/class I complexes. (Paul, W. E., “Chapter 1: The immune system: an introduction,” Fundamental Immunology, 4^{th} Edition, Ed. Paul, W. E., Lippicott-Raven Publishers, Philadelphia (1999)).

The TCR's ligand (i.e., the peptide/MHC protein complex) is created within antigen-presenting cells (APCs). In general, class II MHC molecules bind peptides derived from proteins that have been taken up by the APC through an endocytic process. These peptide-loaded class II molecules are then expressed on the surface of the cell, where they are available to be bound by CD4+ T cells with TCRs capable of recognizing the expressed cell surface complex. Thus, CD4+ T cells are specialized to react with antigens derived from extracellular sources. (Paul, W. E., “Chapter 1: The immune system: an introduction,” Fundamental Immunology, 4^{th} Edition, Ed. Paul, W. E., Lippicott-Raven Publishers, Philadelphia (1999)).

In contrast, class I MHC molecules are mainly loaded with peptides derived from internally synthesized proteins, such as viral proteins. These peptides are produced from cytosolic proteins by proteolysis by the proteosome and are translocated into the rough endoplasmic reticulum. Such peptides, generally nine amino acids in length, are bound into the class I MHC molecules and are brought to the cell surface, where they can be recognized by CD8+ T cells expressing appropriate receptors. (Paul, W. E., “Chapter 1: The immune system: an introduction,” Fundamental Immunology, 4^{th} Edition, Ed. Paul, W. E., Lippicott-Raven Publishers, Philadelphia (1999)).

T cells can also be classified based on their function as helper T cells; T cells involved in inducing cellular immunity; suppressor T cells; and cytotoxic T cells.

Helper T Cells

Helper T cells are T cells that stimulate B cells to make antibody responses to proteins and other T cell-dependent antigens. T cell-dependent antigens are immunogens in which individual epitopes appear only once or a limited number of times such that they are unable to cross-link the membrane immunoglobulin (Ig) of B cells or do so inefficiently. B cells bind the antigen through their membrane Ig, and the complex undergoes endocytosis. Within the endosomal and lysosomal compartments, the antigen is fragmented into peptides by proteolytic enzymes and one or more of the generated peptides are loaded into class II MHC molecules, which traffic through this vesicular compartment. The resulting peptide/class II MHC complex is then exported to the B-cell surface membrane. T cells with receptors specific for the peptide/class II molecular complex recognize this complex on the B-cell surface. (Paul, W. E., “Chapter 1: The immune system: an introduction,” Fundamental Immunology, 4^{th} Edition, Ed. Paul, W. E., Lippicott-Raven Publishers, Philadelphia (1999)).

B-cell activation depends both on the binding of the T cell through its TCR and on the interaction of the T-cell CD40 ligand (CD40L) with CD40 on the B cell. T cells do not constitutively express CD40L. Rather, CD40L expression is induced as a result of an interaction with an APC that expresses both a cognate antigen recognized by the TCR of the T cell and CD80 or CD86. CD80/CD86 is generally expressed by activated, but not resting, B cells so that the helper interaction involving an activated B cell and a T cell can lead to efficient antibody production. In many cases, however, the initial induction of CD40L on T cells is dependent on their recognition of antigen on the surface of APCs that constitutively express CD80/86, such as dendritic cells. Such activated helper T cells can then efficiently interact with and help B cells. Cross-linkage of membrane Ig on the B cell, even if inefficient, may synergize with the CD40L/CD40 interaction to yield vigorous B-cell activation. The subsequent events in the B-cell response, including proliferation, Ig secretion, and class switching (of the Ig class being expressed) either depend or are enhanced by the actions of T cell-derived cytokines (Paul, W. E., “Chapter 1: The immune system: an introduction,” Fundamental Immunology, 4^{th} Edition, Ed. Paul, W. E., Lippicott-Raven Publishers, Philadelphia (1999)).

CD4+ T cells tend to differentiate into cells that principally secrete the cytokines IL-4, IL-5, IL-6, and IL-10 (T_{H2} cells) or into cells that mainly produce IL-2, IFN-γ, and lymphotoxin (T_{H1} cells). The T_{H2} cells are very effective in helping B-cells develop into antibody-producing cells, whereas the T_{H1} cells are effective inducers of cellular immune responses, involving enhancement of microbicidal activity of monocytes and macrophages, and consequent increased efficiency in lysing microorganisms in intracellular vesicular compartments. Although the CD4+ T cells with the phenotype of T_{H2} cells (i.e., IL-4, IL-5, IL-6 and IL-10) are efficient helper cells, T_{H1} cells also have the capacity to be helpers. (Paul, W. E., “Chapter 1: The immune system: an introduction,” Fundamental Immunology, 4^{th} Edition, Ed. Paul, W. E., Lippicott-Raven Publishers, Philadelphia (1999)).

T Cells Involved in Induction of Cellular Immunity

T cells also may act to enhance the capacity of monocytes and macrophages to destroy intracellular microorganisms. In particular, IFN-γ produced by helper T cells enhances several mechanisms through which mononuclear phagocytes destroy intracellular bacteria and parasitism including the generation of nitric oxide and induction of tumor necrosis factor (TNF) production. The T_{H1} cells are effective in enhancing the microbicidal action because they produce IFN-γ. By contrast, two of the major cytokines produced by T_{H2} cells, IL-4 and IL-10, block these activities. (Paul, W. E., “Chapter 1: The immune system: an introduction,” Fundamental Immunology, 4^{th} Edition, Ed. Paul, W. E., Lippicott-Raven Publishers, Philadelphia (1999)).

Suppressor or Regulatory T (Treg) Cells

A controlled balance between initiation and downregulation of the immune response is important to maintain immune homeostasis. Both apoptosis and T cell anergy (a tolerance mechanism in which the T cells are instrinsically functionally inactivated following an antigen encounter (Scwartz, R. H., “T cell anergy,” Annu Rev. Immunol., 21: 305-334 (2003)) are important mechanisms that contribute to the downregulation of the immune response. A third mechanism is provided by active suppression of activated T cells by suppressor or regulatory CD4+ T (Treg) cells. (Reviewed in Kronenberg, M. et al., “Regulation of immunity by self-reactive T cells,” Nature 435: 598-604 (2005)). CD4+ Tregs that constitutively express the IL-2 receptor alpha (IL-2Rα) chain (CD4+CD25+) are a naturally occurring T cell subset that are anergic and suppressive. (Taams, L. S. et 1., “Human anergic/suppressive CD4+CD25+ T cells: a highly differentiated and apoptosis-prone population,” Eur. J. Immunol., 31: 1122-1131 (2001)). Depletion of CD4⁺CD25⁺ Tregs results in systemic autoimmune disease in mice. Furthermore, transfer of these Tregs prevents development of autoimmune disease. Human CD4⁺CD25⁺ Tregs, similar to their murine counterpart, are generated in the thymus and are characterized by the ability to suppress proliferation of responder T cells through a cell-cell contact-dependent mechanism, the inability to produce IL-2, and the anergic phenotype in vitro. Human CD4⁺CD25⁺ T cells can be split into suppressive (CD25high) and nonsuppressive (CD25low) cells, according to the level of CD25 expression. A member of the forkhead family of transcription factors, FOXP3, has been shown to be expressed in murine and human CD4⁺CD25⁺ Tregs and appears to be a master gene controlling CD4⁺CD25⁺ Treg development. (Battaglia, M. et al., “Rapamycin promotes expansion of functional CD4+CD25+Foxp3+ regulator T cells of both healthy subjects and type 1 diabetic patients,” J. Immunol., 177: 8338-8347 (200)).

Cytotoxic T Lymphocytes (CTL)

The CD8+ T cells that recognize peptides from proteins produced within the target cell have cytotoxic properties in that they lead to lysis of the target cells. The mechanism of CTL-induced lysis involves the production by the CTL of perforin, a molecule that can insert into the membrane of target cells and promote the lysis of that cell. Perforin-mediated lysis is enhanced by a series of enzymes produced by activated CTLs, referred to as granzymes. Many active CTLs also express large amounts of fas ligand on their surface. The interaction of fas ligand on the surface of CTL with fas on the surface of the target cell initiates apoptosis in the target cell, leading to the death of these cells. CTL-mediated lysis appears to be a major mechanism for the destruction of virally infected cells.

### Systemic Lupus Erythematosus (SLE)

Lupus or lupus erythematosus is an autoimmune multisystem disorder of unknown etiology characterized by the presence of antinuclear antibodies (ANAs) and associated with inflammation that may be chronic or subacute. Lupus can be of several kinds Systemic lupus erythematosus (SLE) is the most common form of lupus that can affect almost every vital organ in the body, including the joints, skin, kidneys, heart, lungs, blood vessels and brain, and often causes debilitating and potentially life threatening consequences. Discoid lupus erythematosus is a type of lupus that mainly affects the skin. Discoid lupus is associated with red raised rashes on the face or scalp. A small percentage of people who have discoid lupus can also develop SLE. Lupus nephritis is a form of lupus that mainly affects the renal system. Drug-induced lupus is a form of lupus caised by medication. Lupus symptoms persist as long as the drug is administered. Neonatal lupus erythematosus affects newborns of mothers who have lupus or other immune system disorders. (The Patient Education Institute, Inc. ©1995-2009). Profundus lupus erythematosus is characterized by subcutaneous inflammation of adipose tissue (panniculitis) usually on the face with marked lymphocyte infiltration of fat lobules giving rise to deep-seated, firm, rubbery nodules that sometimes become ulcerated.

SLE is a highly heterogenous autoimmune disorder characterized by the prevalence of autoantibodies directed against double-stranded DNA. Worldwide, SLE occurs in approximately 52 per 100,000 individuals and may be highest among individuals of Afro-Caribbean origin at 159 per 100,000. (Danchenko, N. et al., “Epidemiology of systemic lupus erythematosus: a comparison of worldwide disease burden,” Lupus, 15(5): 308-318 (2006). In the United States, SLE is 2.6 times more common in persons of African rather than European origin (19.5 versus 7.4 per 100,000), reflecting a disproportionate ethnic disease burden. For adult-onset SLE, the female:male ratio is 9:1. (Mina, R. et al., “Pediatric lupus—are there differences in presentation, genetics, response to therapy, and damage accrual compared with adult lupus?” Rheumatic Disease Clinics of North America, 36(1): 53-80 (2010); reviewed in Connolly, J. J. et al., “Role of cytokines in systemic lupus erythematosus” Journal of Biomedicineand Biotechnology, 2012: Article ID 798924, pp. 1-17 (2012)).

SLE symptoms may vary from person to person. Almost everyone with SLE has joint pain and swelling, most frequently affecting joints of fingers, hands, wrists and knees. Some patients may develop arthritis. Other common symptoms include, but are not limited to, chest pain, fatigue, fever with no cause, general discomfort, uneasiness or ill feeling, hair loss, mouth sores, sensitivity to sunlight, skin rash (usually over the cheeks and nose bridge), and swollen lymph nodes. In addition, depending on the part of the body affected, other symptoms may include, for example, headaches, numbness, tingling, seizures, vision problems, and personality changes in case lupus affecting brain and nervous system; abdominal pain, nausea and vomiting, in cases of lupus affecting the digestive tract; abnormal heart rhythms (or arrhythmias) in cases of lupus affecting the heart; blood in cough and difficulty in breathing in cases of lupus affecting the lung; and patchy skin, skin rashes, fingers that change color when cold in cases of lupus affecting the skin.

### Assessment of Systemic Lupus Erythematosus (SLE)

Assessment of SLE can be divided into 4 components: 1) diagnosis; 2) monitoring disease activity; 3) assessment of chronic damage; and 4) assessment of the patient's health status throughout the disease course. (Lam, G. K. W. and Petri, M., “Assessment of systemic lupus erythematosus,” Clin. Exp. Rheumatol., 23 (Supp. 39): S120-S132 (2005)).

Diagnosis of SLE

The patient's history, physical examination and lupus-relevant laboratory analyses are critical for accurate diagnosis of SLE. Constitutional symptoms include but are not limited to malaise, fatigue, fever, and unintentional weight loss. However, these symptoms are not specific to SLE alone and may be associated with other etiologies such as fibromyalgia, depression, infection, malignancy, endocrinopathy, or other connective tissue diseases. In addition, environmental triggers such as exposure to ultraviolet radiation, infection, or the use of certain medications (such as Echinacea, sulfonamide antibiotics, minocycline and anti-TNF biologics) may give rise to similar constitutional symptoms. SLE can affect any organ system and can present in differing combinations. (Lam, G. K. W. and Petri, M., “Assessment of systemic lupus erythematosus,” Clin. Exp. Rheumatol., 23 (Supp. 39): S120-S132 (2005)).

The most frequent manifestations include but are not limited to: arthritis, arthralgias, skin lesions, renal disease, Raynaud's phenomenon, central nervous system involvement, gastrointestinal symptoms, pleurisy, pericarditis, lymphadenopathy, nephritic syndrome, lung involvement, thrombophlebitis, myositis, and myocarditis. Arthritis and arthralgias are the most common presenting manifestations of SLE. Any joint may be affected, but the small joints of the hands and wrists, and occasionally knees are typically involved. Skin manifestations are also common. They are usually classified based on their appearance and duration as acute, subacute and chronic. Malar rash, usually triggered by exposure to ultraviolet light, is the most common acute lesion that is characterized by erythema and elevation in a butterfly rash around the nose bridge. Renal disease is also prevalent in a majority of SLE patients, a form of lupus known as lupus nephritis. Neurological and psychiatric manifestations have also been reported but are difficult to estimate because most such symptoms are non-specific, such as headache, depression, and anxiety. Neurological features may include but are not limited to seizures, stroke, movement disorders (chorea), intractable headaches, and cranial neuropathy. (Lam, G. K. W. and Petri, M., “Assessment of systemic lupus erythematosus,” Clin. Exp. Rheumatol., 23 (Supp. 39): S120-S132 (2005)).

Neuropsychiatric manifestations are a significant cause of morbidity in SLE. The American College of Rheumatology (ACR) formulated case definitions for neuropsychiatric SLE syndromes that include cognitive dysfunction in patients with difficulties in attention, concentration, memory and word-finding. (Liang, M. H. et al., “The American College of Rheumatology nomenclature and case definitions for neuropsychiatric lupus syndromes,” Arth. Rheum. 42(4): 599-608 (1999)). Attention deficit and hyperactivity disorder (ADHD), may be an early sign of cognitive impairment and progressive mania or depression. (Biederman, J. et al., “Adult psychiatric outcomes of girls with attention deficit hyperactivity disorder: 11-year follow-up in a longitudinal case-control study,” Am. J. Psychiatry, 167(4): 409-417 (2010).

SLE is an autoimmune disease in which autoantibodies are frequently targeted against intracellular antigens of the cell nucleus (including both double-stranded (ds−) and single-stranded (ss−) DNA), histones, and extractable nuclear antigens (ENAs). Many of these autoantibodies may not be specific to SLE and may be produced non-specifically as a result of polyclonal B cell activation. A majority of lupus-relevant laboratory tests focus on the detection of antinuclear antibodies (ANA), anti-DNA antibodies, anihistone antibodies, anti-ENA antibodies, Ribosomal P antibodies, antiphospholipid antibodies, acute phase cytokines, complement, anti-C1q antibodies, anti-endothelial cell antibodies, antineutrophil cytoplasmic antibodies, etc. No test or test panel can currently perform all these tasks. Therefore, a variety of laboratory tests are usually necessary for accurate diagnosis of SLE. (Egner, W., “The use of laboratory test in the diagnosis of SLE,” J. Clin. Pathol., 53: 424-432 (2000)).

Serologically, the production of various autoantibodies is the immunopathologic basis of disease. As an initial step, a positive ANA implicates autoimmunity. (Lam, G. K. W. and Petri, M., “Assessment of systemic lupus erythematosus,” Clin. Exp. Rheumatol., 23 (Supp. 39): S120-S132 (2005)). ANA are seen in 90-95% of patients with SLE. ANA is traditionally detected by indirect immunofluorescence (IF) assay in which the antibodies of the patients' sera that bind to the nucleus of Hep-2 human epipharynx carcinoma cells are detected by fluorescein isothiocyanate (FITC)-conjugated anti-human IgG, using fluorescence microscopy. The IF technique provides information on the pattern of fluorescence (such as homogenous, peripheral, nucleolar, or speckled) that is relevant for antigen specificity and has been associated with autoimmune disease subsets. (Tan, E. M. et al., “Range of antinuclear antibodies in “healthy” individuals,” Arthritis Rheum, 40: 1601-1611 (1997)). Flow cytometry with autoantigen-coated fluorescent beads (FB), also commonly referred to as Reflex ANA, offers many advantages over the IF technique, such as simultaneous testing for recognition of several antigens, automation, cost effectiveness, and high sensitivity. (Shovman, O. et al., “Multiplexed AtheNA multi-lyte immunoassay for ANA screening in autoimmune diseases,” Autoimmunity, 38: 105-109 (2005)). However, an analysis of the two methods for ANA detection showed that the IF assay has superior sensitivity for detection of ANA. (Bonilla, E. et al., “Immunofluorescence microscopy is superior to fluorescent beads for detection of antinuclear antibody reactivity in systemic lupus erythematosus patients,” Clin. Immunol., 124: 18-21 (2007)).

While the hallmark of SLE is the presence of antinuclear antibodies (ANA), a number of laboratory abnormalities may be used to characterize lupus. Antibodies to double-stranded DNA (anti-dsDNA) are found in 40-60% of SLE patients. (Lam, G. K. W. and Petri, M., “Assessment of systemic lupus erythematosus,” Clin. Exp. Rheumatol., 23 (Supp. 39): S120-S132 (2005)). The Farr assay is used to quantify the amount of anti-double-stranded (anti-ds) DNA antibodies in serum. It is a radioimmunoassay based on ammonium sulfate precipitation to separate DNA/anti-DNA complexes from free radiolabelled DNA in a liquid phase. The Farr assay detects high affinity anti-ds DNA antibodies with no distinction of isotypes. (Rouquette, A. M. and Desgruelles, C., “Detection of antobodies to dsDNA: an overview of laboratory assays,” Lupus, 15: 403-407 (2006)).

Antiphospholipid antibodies may also be found in lupus (50%) and can cause venous and arterial thromboses, as well as recurrent fetal loss. Assessment is by detection of antibodies to cardiolipin or to beta-2 glycoprotein 1, or by the presence of a lupus anticoagulant. (Lam, G. K. W. and Petri, M., “Assessment of systemic lupus erythematosus,” Clin. Exp. Rheumatol., 23 (Supp. 39): S120-S132 (2005)).

Autoantibodies lead to the formation of immune complexes, which activate and consume complement. Hence, measuring levels of C3, C4, or total hemolytic complement CH50 may be helpful in the diagnosis of lupus. (Lam, G. K. W. and Petri, M., “Assessment of systemic lupus erythematosus,” Clin. Exp. Rheumatol., 23 (Supp. 39): S120-S132 (2005); Schur, P. H. and Snadson, J., “Immunologic factors and clinical activity in systemic lupus erythematosus,” New Engl. J. Med., 278: 533-538 (1968))).

Criteria for Classification of Systemic Lupus Erythematosus

The Diagnostic and Therapeutic Criteria Committee of the American College of Rheumatology (ACR) published revised criteria for the classification of systemic lupus erthematosus (SLE) in 1982 and then an update of the revised criteria in 1997 (Tan, E. M. et al., “The 1982 revised criteria for the classification of systemic lupus erythematosus,” Arthritis Rheum., 25: 1270-1277 (1982); Hochberg, M. C., “Updating the American College of Rheumatology revised criteria for the classification of systemic lupus erythematosus,” Arthritis Rheum., 40(9): 1725 (1997)). Table 1 lists the eleven updated ACR diagnostic criteria for SLE.

TABLE 1
1997 Update of the 1982 American College of Rheumatology Revised
Criteria for Classification of Systemic Lupus Erythematosus (SLE)
Table 1: 1997 Update of 1982 ACR Diagnotic Criteria for SLE

| CRITERION | DEFINITION |
|---|---|
| 1. Malar Rash | Fixed erythema, flat or raised, over the malar eminences, tending to spare |
| | the nasolabial folds |
| 2. Discoid Rash | Erythematous raised patches with adherent keratotic scaling and |
| | follicular plugging; atrophic scarring may occur in older lesions |
| 3. Photosensitivity | Skin rash as a result of unusual reaction to sunlight, by patient history or |
| | physician observation |
| 4. Oral Ulcers | Oral or nasopharyngeal ulceration, usually painless, observed by |
| | physician |
| 5. Nonerosive | Involving 2 or more peripheral joints, characterized by tenderness, |
| Arthritis | swelling, or effusion |
| 6. Pleuritis or | a. Pleuritis--convincing history of pleuritic pain or rubbing heard by |
| Pericarditis | a physician or evidence of pleural effusion |
| | OR |
| | b. Pericarditis--documented by electrocardigram or rub or evidence |
| | of pericardial effusion |
| 7. Renal Disorder | a. Persistent proteinuria >0.5 grams per day or > than 3+ if |
| | quantitation not performed |
| | OR |
| | b. Cellular casts--may be red cell, hemoglobin, granular, tubular, or |
| | mixed |
| 8. Neurologic | a. Seizures--in the absence of offending drugs or known metabolic |
| Disorder | derangements; e.g., uremia, ketoacidosis, or electrolyte imbalance |
| | OR |
| | b. Psychosis--in the absence of offending drugs or known metabolic |
| | derangements, e.g., uremia, ketoacidosis, or electrolyte imbalance |
| 9. Hematologic | a. Hemolytic anemia--with reticulocytosis |
| Disorder | OR |
| | b. Leukopenia--<4,000/mm³ on ≧2 occasions |
| | OR |
| | c. Lymphopenia--<1,500/mm³ on ≧2 occasions |
| | OR |
| | d. Thrombocytopenia--<100,000/mm³ in the absence of offending |
| | drugs |
| 10. Immunologic | a. Anti-DNA: antibody to native DNA in abnormal titer |
| Disorder | OR |
| | b. Anti-Sm: presence of antibody to Sm nuclear antigen |
| | OR |
| | c. Positive finding of antiphospholipid antibodies on: |
| | 1. an abnormal serum level of IgG or IgM |
| | anticardiolipin antibodies, |
| | 2. a positive test result for lupus anticoagulant using |
| | a standard method, or |
| | 3. a false-positive test result for at least 6 months |
| | confirmed by Treponema pallidum |
| | immobilization or fluorescent treponemal |
| | antibody absorption test |
| 11. Positive | An abnormal titer of antinuclear antibody by immunofluorescence or an |
| Antinuclear | equivalent assay at any point in time and in the absence of drugs |
| Antibody | |

Standardized Measures of Disease Activity in SLE

The term “disease activity” as used herein is defined as reversible manifestations of the underlying inflammatory process in systemic lupus erthematosus. It is a reflection of the type and severity of organ involvement at each point in time. (Bombardier, C. et al., “Derivaion of the SLEDAI: a disease activity index for lupus patients,” Arthritis Rheum., 35(6): 630-640 (1992)). Because no single measure can describe status in all SLE patients, standardized indices for assessing SLE disease activity have been described. Of these, the SLE Disease Activity Index (SLEDAI), and the British Isles Lupus Assessment Group (BILAG) are the most common. (Lam, G. K. W. and Petri, M., “Assessment of systemic lupus erthematosus,” Clin. Exp. Rheumatol., 23 (Supp. 39): S120-S132 (2005)).

SLEDAI offers an assessment tool for assessing disease activity in SLE. Twenty-four features that are attributed to lupus are listed, with a weighted score given to any one that is present. The more serious manifestations (such as renal, neurologic, and vasculitis) are weighted more than others (such as cutaneous manifestations). The maximum possible score is 105. (Lam, G. K. W. and Petri, M., “Assessment of systemic lupus erthematosus,” Clin. Exp. Rheumatol., 23 (Supp. 39): S120-S132 (2005)). Table 2 shows the systemic lupus erythematosus disease activity index (SLEDAI) modified from Bombardier, C. et al., “Derivation of the SLEDAI: a disease activity index for lupus patients,” Arthritis Rheum., 35(6): 630-640 (1992).

TABLE 2
Systemic Lupus Erythematosus Disease Activity Index (SLEDAI)
Table 2: SLEDAI

| SCORE | DESCRIPTOR | DEFINITION |
|---|---|---|
| 8 | Seizure | Recent onset. |
| | | Exclude metabolic, infectious, or drug-related causes. |
| 8 | Psychosis | Altered ability to function in normal activity due to severe |
| | | disturbance in the perception of reality. Includes |
| | | hallucinations; incoherence; marked loose associations; |
| | | impoverished thought content; marked illogical thinking; |
| | | bizarre, disorganized or catatonic behavior. |
| | | Exclude the presence of uremia and offending drugs. |
| 8 | Organic brain | Altered mental function with impaired orientation or |
| | syndrome | impaired memory or syndrome other intellectual function, |
| | | with rapid onset and fluctuating clinical features. |
| | | Includes a clouding of consciousness with a reduced |
| | | capacity to focus and an inability to sustain attention on |
| | | environment, and at least two of the following: perceptual |
| | | disturbance, incoherent speech, insomnia or daytime |
| | | drowsiness, increased or decreased psychomotor activity. |
| | | Exclude metabolic, infectious, and drug-related causes. |
| 8 | Visual | Retinal changes from systemic lupus erythematosus: cytoid |
| | | bodies, retinal hemorrhages, serous exudates or |
| | | hemorrhages in the choroid, optic neuritis (not due to |
| | | hypertension, drugs, or infection). |
| 8 | Cranial nerve | New onset of a sensory or motor neuropathy involving a |
| | | cranial nerve. |
| 8 | Lupus headache | Severe, persistent headache; may be migranous; |
| | | unresponsive to narcotics |
| 8 | Cerebrovascular | New syndrome. |
| | accident | Exclude arteriosclerosis. |
| 8 | Vasculitis | Ulceration, gangrene, tender finger nodules, periungual |
| | | infarction, splinter hemorrhages. Vasculitis confirmed by |
| | | biopsy or angiogram |
| 4 | Arthritis | More than 2 joints with pain and signs of inflammation. |
| 4 | Myositis | Proximal muscle aching or weakness associated with |
| | | elevated creatine phosphokinase/aldolase levels, |
| | | electromyographic changes, or a biopsy showing myositis. |
| 4 | Casts | Heme, granular, or erythrocyte. |
| 4 | Hematuria | More than 5 erythrocytes per high power field. |
| | | Exclude other causes (stone, infection). |
| 4 | Proteinuria | More than 0.5 grams of urinary protein excreted per 24 h. |
| | | New onset or recent increase of >0.5 g/24 h. |
| 4 | Pyuria | More than 5 leukocytes per high-power field. |
| | | Exclude infection. |
| 2 | New malar rash | New onset or recurrence of an inflammatory type of rash. |
| 2 | Alopecia | New or recurrent. Apatch of abnormal, diffuse hair loss. |
| 2 | Mucous membranes | New onset or recurrence of oral or nasal ulcerations. |
| 2 | Pleurisy | Pleuritic chest pain with pleural rub or effusion, or pleural |
| | | thickening. |
| 2 | Pericarditis | Pericardial pain with at least one of rub or effusion. |
| | | Confirmation by electro- or echocardiography. |
| 2 | Low complement | A decrease in CH50, C3, or C4 level (to less than the lower |
| | | limit of the laboratory-determined normal range). |
| 2 | Increased DNA | More than 25% binding by Farr assay (to >the upper limit of |
| | binding | the laboratory-determined normal range, e.g. 25%). |
| 2 | Fever | More than 38° C. after the exclusion of infection. |
| 2 | Thrombocytopenia | Fewer than 100,000 platelets |
| 2 | Leukopenia | Leukocyte count of <3000/mm³ (not due to drugs) |

The BILAG index is more comprehensive than the SLEDAI, recording clinical disease activity in 8 different organ systems for a total of 101 items. Each item is measured qualitatively by clinical observation (yes/no, improving/same/worse/new) or quantitatively by measuring hematologic and renal lab values. Based on these items, each of the 8 organ systems is allocated an alphabetical score of A (most active), B (moderate activity), C (minor activity), D (stable), or E (never present). Normally, a total score is not calculated. However, it can be converted into a disease activity scale by assigning points to the alphabetical score: A=9, B=3, C=1, D=0, E=0, with a maximum potential score of 72. (Lam, G. K. W. and Petri, M., “Assessment of systemic lupus erthematosus,” Clin. Exp. Rheumatol., 23 (Supp. 39): S120-S132 (2005)). The classical BILAG index was subsequently updated as the BILAG 2004 index. Table 3 shows the BILAG 2004 index adapted from Isenberg, D. A. et al., “BILAG 2004. Development and initial validation of an updated version of the British Isles Lupus Assessment Group's disease activity index for patients with systemic lupus erythematosus,” Rheumatology, 44: 902-906 (2005). According to the BILAG 2004 index, all features must be attributable to SLE and refer to the last 4 weeks compared with the previous 4 weeks; in some manifestations, it may be difficult to differentiate SLE from other causes as there may not be any specific test and the decision would then lies with the physician's judgement on the balance of probabilities. The term “improvement” is defined as (a) the amount that is sufficient for consideration of reduction in therapy; and (b) that which is present ≧2 weeks of the previous 4 weeks. Scoring for features is indicated as: (0) not present; (1) Improving; (2) Same; (3) Worse; (4) New.

TABLE 3
British Isles Lupus Assessment Group 2004 (BILAG 2004) Index
Table 3. BILAG 2004 Index

| CATEGORY | DESCRIPTOR | DEFINITION |
|---|---|---|
| CONSTITUTIONAL | 1. Pyrexia | temperature >37.5° C. documented |
| | 2. Unintentional weight loss >5% | |
| | 3. Lymphadenopathy | palpable lymph node more than 1 cm diameter |
| | 4. Fatigue or malaise or | |
| | lethargy | |
| | 5. Anorexia | |
| MUCOCUTANEOUS | 6. Severe eruption | >18% body surface area or bullous includes |
| | | discoid lesion |
| | | body surface area (BSA) is defined using the |
| | | rules of nines (used to assess extent of burns) |
| | | as follows: |
| | | palm (excluding fingers) = 1% BSA |
| | | each lower limb = 18% BSA |
| | | each upper limb = 9% BSA |
| | | torso (front) = 18% BSA |
| | | torso (back) = 18% BSA |
| | | head = 9% BSA |
| | | genital (male) = 1% BSA |
| | 7. Mild eruption | ≦18% body surface area includes discoid |
| | | lesion |
| | 8. Angio-oedema | potentially life-threatening eg: stridor |
| | 9. Severe mucosal ulceration | disabling extensive &/or deep ulceration |
| | 10. Mild mucosal ulceration | localised non-disabling ulceration |
| | 11. Severe panniculitis | any one: affecting the face |
| | | >9% body surface area |
| | | threatens integrity of epithelium &/or |
| | | subcutaneous tissue |
| | 12. Mild panniculitis | ≦9% body surface area and does not fulfil any |
| | | criteria for severe panniculitis |
| | 13. Cutaneous | resulting in gangrene or ulceration or skin |
| | vasculitis/thrombosis | infarction |
| | 14. Digital infarct/nodular | localised single or multiple infarct(s) over |
| | vasculitis | digit(s) or tender erythematous nodule(s) |
| | 15. Severe alopecia | clinically detectable diffuse or patchy hair loss |
| | | with scalp inflammation |
| | 16. Mild alopecia | not clinically detectable and little/no scalp |
| | | inflammation (may be diffuse & must be |
| | | spontaneous) |
| | 17. Peri-ungual erythema or | |
| | chilblains | |
| | 18. Splinter haemorrhages | |
| NEUROPSYCHIATRIC | 19. Aseptic meningitis | criteria (all): acute/subacute onset |
| | | headache |
| | | photophobia |
| | | neck stiffness |
| | | fever |
| | | signs of meningeal irritation |
| | | abnormal CSF but negative cultures |
| | | exclude CNS/meningeal infection, intracranial |
| | | haemorrhage |
| | 20. Cerebral vasculitis | should be present with features of vasculitic in |
| | | another system and supportive imaging &/or |
| | | biopsy findings |
| | 21. Demyelinating syndrome | discrete white matter lesion with associated |
| | | neurological deficit not recorded elsewhere |
| | | there must have been at least one previously |
| | | recorded event |
| | | exclude multiple sclerosis |
| | 22. Myelopathy | acute onset of rapidly evolving paraparesis or |
| | | quadriparesis and/or sensory level |
| | | exclude intramedullary and extramedullary |
| | | space occupying lesion |
| | 23. Acute confusional state | acute disturbance of consciousness or level of |
| | | arousal with reduced ability to focus, maintain |
| | | or shift attention |
| | | includes hypo- and hyperaroused states and |
| | | encompasses the spectrum from delirium to |
| | | coma |
| | 24. Psychosis | delusion or hallucinations |
| | | does not occur exclusively during course of a |
| | | delirium |
| | | exclude drugs, substance abuse, primary |
| | | psychotic disorder |
| | 25. Acute inflammatory | Criteria: |
| | demyelinating | progressive polyradiculoneuropathy |
| | polyradiculoneuropathy | loss of reflexes |
| | | symmetrical involvement |
| | | increased CSF protein without |
| | | pleocytosis |
| | | supportive abnormal nerve conduction |
| | | study |
| | 26. Mononeuropathy | nerve conduction study not essential |
| | (single/multiplex) | |
| | 27. Cranial neuropathy | except optic neuropathy which is classified |
| | | elsewhere |
| | 28. Plexopathy | disorder of brachial or lumbosacral plexus |
| | | resulting in neurological deficit not |
| | | corresponding to territory of single root or |
| | | nerve |
| | | positive electrophysiology study required |
| | 29. Polyneuropathy | symmetrical distal sensory and/or motor deficit |
| | | positive electrophysiology study required |
| | 30. Seizure disorder | independent description of seizure by reliable |
| | | witness |
| | 31. Status epilepticus | a seizure or series of seizures lasting ≧30 |
| | | minutes without full recovery to baseline |
| | 32. Cerebrovascular disease | any one with supporting imaging: |
| | (not due to vasculitis) | stroke syndrome |
| | | transient ischaemic attack |
| | | intracranial haemorrhage |
| | | exclude hypoglycaemia, cerebral sinus |
| | | thrombosis, vascular malformation, tumour, |
| | | abscess |
| | | cerebral sinus thrombosis not included as |
| | | definite thrombosis not considered part of lupus |
| | | activity |
| | 33. Cognitive dysfunction | significant deficits in any cognitive functions: |
| | | simple attention |
| | | complex attention |
| | | memory |
| | | visual-spatial processing |
| | | language |
| | | reasoning/problem solving |
| | | psychomotor speed |
| | | executive functions |
| | | neuropsychological testing should be done if |
| | | possible or corroborating history from third |
| | | party that it is interfering with daily activities |
| | | exclude substance abuse |
| | 34. Movement disorder | exclude drugs |
| | 35. Autonomic disorder | any one: |
| | | fall in blood pressure to standing >30/ |
| | | 15 mmHg (systolic/diastolic) |
| | | increase in heart rate to standing ≧30 |
| | | bpm |
| | | loss of heart rate variation with |
| | | respiration |
| | | (max-min <15 bpm, |
| | | expiration:inspiration |
| | | ratio <1.2, Valsalva ratio <1.4) |
| | | loss of sweating over body and limbs |
| | | (anhidrosis) by sweat test |
| | | exclude drugs and diabetes mellitus |
| | 36. Cerebellar ataxia | |
| | 37. Severe headache | disabling headache unresponsive to narcotic |
| | (unremitting) | analgesia & lasting ≧3 days |
| | | exclude intracranical space occupying lesion |
| | | and CNS infection |
| | 38. Migraine with/without aura | recurrent attacks of headache lasting 4-72 |
| | | hours |
| | | may be preceded by neurological aura (lasting |
| | | up to 1 hour) |
| | 39. Tension headache | recurrent episodes of headaches lasting minutes |
| | | to days |
| | 40. Cluster headache | attacks of severe unilateral headache lasting |
| | | 15-180 minutes |
| | | attacks at least once every other day and up to 8 |
| | | times a day |
| | | attacks occur in clusters (series of weeks or |
| | | months) separated by remissions of usually |
| | | months or years |
| | 41. Headache from IC | exclude cerebral sinus thrombosis |
| | hypertension | |
| | 42. Mood disorder | prominent & persistent disturbance in mood |
| | (depression/mania) | characterised by depressed mood or markedly |
| | | diminished interest or pleasure in almost all |
| | | activities or elevated, expansive or irritable |
| | | mood |
| | | should result in significant distress or impaired |
| | | functioning |
| | 43. Anxiety disorder | prominent anxiety, panic disorder, panic attacks |
| | | or obsessions or compulsions resulting in |
| | | clinically significant distress or impaired |
| | | functioning |
| MUSCULOSKELETAL | 44. Definite myositis | ≧3 Bohan & Peter criteria*: |
| | | proximal muscle weakness |
| | | elevated muscle enzymes |
| | | positive muscle biopsy |
| | | abnormal EMG |
| | | *Bohan, A. and Peter, J. B., “Polymyositis and |
| | | dermatomyositis,” N. Engl. J. Med. 292: 344-347, |
| | | 403-407 (1975) |
| | 45. Incomplete myositis | 2 Bohan & Peter criteria* |
| | | *Bohan, A. and Peter, J. B., “Polymyositis and |
| | | dermatomyositis,” N. Engl. J. Med. 292: 344-347, |
| | | 403-407 (1975) |
| | 46. Severe polyarthritis | observed active synovitis ≧2 joints with |
| | | significant impairment of activities of daily |
| | | living and has been present on several days |
| | | (cumulatively) over the last 4 weeks |
| | 47. Arthritis or Tendonitis | tendonitis or active synovitis ≧1 joint with |
| | | some impairment of function, which has been |
| | | present on several days over the last 4 weeks |
| | 48. Arthralgia or Myalgia | inflammatory joint or muscle pain which does |
| | | not fulfil the above criteria for arthritis or |
| | | myositis |
| CARDIORESPIRATORY | 49. Mild myocarditis | inflammation of myocardium with raised |
| | | cardiac enzymes &/or ECG changes and |
| | | without resulting cardiac failure, arrhythmia or |
| | | valvular dysfunction |
| | 50. Cardiac failure | cardiac failure due to myocarditis or non- |
| | | infective inflammation of endocardium or |
| | | cardiac valves (endocarditis) |
| | 51. Arrhythmia | arrhythmia (except sinus tachycardia) due to |
| | | myocarditis or non-infective inflammation of |
| | | endocardium or cardiac valves (endocarditis) |
| | 52. New valvular dysfunction | new cardiac valvular dysfunction due to |
| | | myocarditis or non-infective inflammation of |
| | | endocardium or cardiac valves (endocarditis) |
| | 53. Mild serositis (pleuro- | in absence of cardiac tamponade or pleural |
| | pericardial pain) | effusion with dyspnoea |
| | 54. Cardiac tamponade | |
| | 55. Pleural effusion with | |
| | dyspnoea | |
| | 56. Pulmonary | inflammation of pulmonary vasculature with |
| | haemorrhage/vasculitis | haemoptysis &/or dyspnoea &/or pulmonary |
| | | hypertension |
| | | supporting imaging &/or histological diagnosis |
| | 57. Interstitial | radiological features of alveolar infiltration not |
| | alveolitis/pneumonitis | due to infection or haemorrhage reduced |
| | | corrected gas transfer Kco (<70% normal) |
| | 58. Shrinking lung syndrome | reduced lung volumes (<70% predicted) in |
| | | presence of normal corrected gas transfer Kco |
| | | with dysfunctional diaphragmatic movements |
| | 59. Aortitis | inflammation of aorta with or without |
| | | dissection with supporting imaging |
| | | abnormalities |
| | | accompanied by >10 mmHg difference in |
| | | blood pressure (BP) between arms &/or |
| | | claudication of extremities &/or vascular bruits |
| | 60. Coronary vasculitis | inflammation of coronary vessels with |
| | | radiographic evidence of non-atheromatous |
| | | narrowing, obstruction or aneurismal changes |
| GASTROINTESTINAL | 61. Peritonitis | serositis presenting as acute abdomen with |
| | | rebound/guarding |
| | 62. Serositis | not presenting as acute abdomen |
| | 63. Lupus enteritis or colitis | vasculitis or inflammation of small or large |
| | | bowel with supportive imaging &/or biopsy |
| | | findings |
| | 64. Malabsorption | diarrhoea with abnormal D- xylose absorption |
| | | test or increased faecal fat excretion after |
| | | exclusion of coeliac's disease (poor response to |
| | | gluten-free diet) and gut vasculitic |
| | 65. Protein-losing enteropathy | diarrhea with hypoalbuminaemia or increased |
| | | fecal excretion of iv radiolabeled albumin after |
| | | exclusion of gut vasculitic |
| | 66. Intestinal pseudo- | subacute intestinal obstruction due to intestinal |
| | obstruction | hypomotility |
| | 67. Hepatitis | raised transaminases in absence of |
| | | autoantibodies specific to autoimmune hepatitis |
| | | (eg: anti-smooth muscle, anti-liver cytosol 1) |
| | | &/or biopsy appearance of chronic active |
| | | hepatitis |
| | 68. Acute cholecystitis | after exclusion of gallstones and infection |
| | 69. Acute pancreatitis | |
| OPHTHALMIC | 70. Orbital inflammation | |
| | 71. Severe keratitis | sight threatening |
| | | includes: |
| | | corneal melt |
| | | peripheral ulcerative keratitis |
| | 72. Mild keratitis | not sight threatening |
| | 73. Anterior uveitis | |
| | 74. Severe posterior uveitis | sight-threatening &/or retinal vasculitic |
| | &/or retinal vasculitis | not due to vaso-occlusive disease |
| | 75. Mild posterior uveitis &/or | not sight-threatening |
| | retinal vasculitis | not due to vaso-occlusive disease |
| | 76. Episcleritis | |
| | 77. Severe scleritis | necrotising anterior scleritis |
| | | anterior &/or posterior scleritis requiring |
| | | systemic steroids/immunosuppression &/or not |
| | | responding to NSAIDs |
| | 78. Mild scleritis | anterior &/or posterior scleritis not requiring |
| | | systemic steroids |
| | | excludes necrotising anterior scleritis |
| | 79. Retinal/choroidal vaso- | includes: |
| | occlusive disease | retinal arterial & venous occlusion |
| | | serous retinal &/or retinal pigment |
| | | epithelial detachments secondary to |
| | | choroidal vasculopathy |
| | 80. Isolated cotton-wool spots | also known as cytoid bodies |
| | 81. Optic neuritis | excludes anterior ischaemic optic neuropathy |
| | 82. Anterior ischaemic optic | visual loss with pale swollen optic disc due to |
| | neuropathy | occlusion of posterior ciliary arteries |
| RENAL | 83. Systolic blood pressure | |
| | 84. Diastolic blood pressure | |
| | 85. Accelerated hypertension | blood pressure rising to >170/110 mmHg within |
| | | 1 month with grade 3 or 4 Keith-Wagener-Barker |
| | | retinal changes (flame-shaped haemorrhages or |
| | | cotton-wool spots or papilloedema) |
| | 86. Urine dipstick | |
| | 87. Urine albumin-creatinine | on freshly voided urine sample |
| | ratio | |
| | 88. Urine protein-creatinine ratio | on freshly voided urine sample |
| | 89. 24 hour urine protein | |
| | 90. Nephrotic syndrome | criteria: |
| | | heavy proteinuria (>50 mg/kg/day or |
| | | >3.5 g/day or protein-creatinine ratio >350 mg/mmol |
| | | or albumin-creatinine ratio |
| | | >350 mg/mmol) |
| | | hypoalbuminaemia |
| | | oedema |
| | 91. Plasma/Serum creatinine | |
| | 92. GFR | MDRD formula: |
| | | GFR = 170 × [serum creatinine(mg/dl)^{-0.999} × |
| | | [age]^{-0.176} × [serum urea(mg/dl]^{-0.17} × |
| | | [serum albumin(g/dl)]^{0.318} × [0.762 if |
| | | female] × [1.180 if black] |
| | | conversion: |
| | | serum creatinine − mg/dl = (μmol/l)/88.5 |
| | | serum urea − mg/dl = (mmol/l) × 2.8 |
| | | creatinine clearance not recommended as it is not |
| | | reliable |
| | 93. Active urinary sediment | Uncentrifuged specimen: pyuria (>5 WCC/hpf), |
| | | haematuria (>5 RBC/hpf) or red cell casts in |
| | | absence of other causes |
| | 94. Histology of active nephritis | WHO Class III, IV or V* |
| | | within last 3 months or since previous |
| | | assessments if seen less than 3 months ago |
| | | glomerular sclerosis without inflammation not |
| | | counted |
| | | *Goldbus, J. and McClune, W. J., “Lupus |
| | | nephritis: Classification, prognosis, |
| | | immunopathogenesis and treatment,” Rheum. |
| | | Dis. Clin. North Am., 20: 213-242 (1994) |
| HAEMATOLOGY | 95. Haemoglobin | |
| | 96. White cell count | |
| | 97. Neutrophil count | |
| | 98. Lymphocyte count | |
| | 99. Platelet count | |
| | 100. Evidence of active | positive Coomb's test & evidence of haemolysis |
| | haemolysis | (raised bilirubin or raised reticulocyte count or |
| | | reduced haptoglobulins) |
| | 101. Isolated positive Coomb's | |
| | test | |

Other standardized measures of disease activity that can be incorporated into routine clinical care and provide quick snapshots of a patient's physical status include, but are not limited to, the Systemic Lupus Activity Measure (SLAM), the Lupus Activity Index (LAI), and the European Consensus Lupus Activity Measurement (ECLAM). (Lam, G. K. W. and Petri, M., “Assessment of systemic lupus erthematosus,” Clin. Exp. Rheumatol., 23 (Supp. 39): S120-S132 (2005)).

Assessment of Chronic Damage of SLE

Although survival rate with SLE has increased over time with improved therapies, a substantial amount of organ damage may accumulate throughout a patient's life. The Systemic Lupus International Collaborating Clinics (SLICC) and ACR damage index (SLICC/ACR damage index) provides reliable measures for organ damage after the diagnosis of lupus. The SLICC/ACR damage index complements the other measures of disease activity and is an important outcome measure. It is usually completed (or updated yearly). (Lam, G. K. W. and Petri, M., “Assessment of systemic lupus erthematosus,” Clin. Exp. Rheumatol., 23 (Supp. 39): S120-S132 (2005)).

Assessment of Health Status of Patients

While the diagnosis, assessment of disease activity and assessment of chronic damage of SLE is performed by the physician, a patient's own perception of his or her health and quality of life is an equally important component in the overall assessment of SLE. The Short-Form 36 (SF-36) is the most widely used and comprehensive index for this purpose. The SF-36 includes one multi-item scale that assesses 8 health concepts: 1) limitations in physical activities because of health problems; 2) limitations in social activities because of physical or emotional problems; 3) limitations in usual role activities because of physical health problems; 4) bodily pain; 5) general mental health; 6) limitations in usual role activities because of emotional problems; 7) vitality (energy and fatigue); and 8) general health perceptions. (Lam, G. K. W. and Petri, M., “Assessment of systemic lupus erthematosus,” Clin. Exp. Rheumatol., 23 (Supp. 39): S120-S132 (2005)).

Fatigue, a nonspecific symptom that is highly prevalent among patients in primary care, is not only an important component of many diseases or disorders, including SLE, but can also play a substantial role in healthy populations. Fatigue is considered the most disabling symptom in a majority of SLE patients. (Krupp, L. B. et al., “A study of fatigue in systemic lupus erythematosus,” J. Rheumatol., 17: 1450-1452 (1990)). The Fatigue Assessment Scale (FAS) is a 10-item unidimensional subjective fatigue scale that measures chronic fatigue. Table 4 lists the 10 statements of the Fatigue Assessment Scale that refer to how one feels. For each statement, one chooses one out of five answer categories varying from “never” to “always: 1=never; 2=sometimes; 3=regularly; 4=often; and 5=always. (Michielsen, H. J. et al., “Psychometric qualities of a brief self-rated fatigue measure: The Fatigue Assessment Scale,” Journal of Psychosomatic Research, 54: 345-352 (2003)).

TABLE 4
The Fatigue Assessment Scale (FAS)
Table 4: The Fatigue Assessment Scale (FAS)

| | Answer Categories | | | | |
|---|---|---|---|---|---|
| Statements | Never | Sometimes | Regularly | Often | Always |
| 1. I am bothered by | 1 | 2 | 3 | 4 | 5 |
| fatigue. | | | | | |
| 2. I get tired very | 1 | 2 | 3 | 4 | 5 |
| quickly. | | | | | |
| 3. I don't do much | 1 | 2 | 3 | 4 | 5 |
| during the day. | | | | | |
| 4. I have enough | 1 | 2 | 3 | 4 | 5 |
| energy for everyday | | | | | |
| life. | | | | | |
| 5. Physically, I feel | 1 | 2 | 3 | 4 | 5 |
| exhausted. | | | | | |
| 6. I have problems | 1 | 2 | 3 | 4 | 5 |
| starting things. | | | | | |
| 7. I have problems | 1 | 2 | 3 | 4 | 5 |
| thinking clearly. | | | | | |
| 8. I feel no desire to | 1 | 2 | 3 | 4 | 5 |
| do anything. | | | | | |
| 9. Mentally, I feel | 1 | 2 | 3 | 4 | 5 |
| exhausted | | | | | |
| 10. When I am | 1 | 2 | 3 | 4 | 5 |
| doing something, | | | | | |
| I can concentrate | | | | | |
| very well. | | | | | |

As for neuropsychiatric manifestations in the form of attention deficit and hyperactivity disorder (ADHD), the World Health Organization (WHO) Adult ADHD Self-Report Scale (ASRS) provides a subjective self-report screening scale of adult ADHD. The ASRS is an 18-item scale that is used to assess the current status of the 18 DSM-IV symptoms of ADHD in adults. (Kessler, R. C. et al., “The World Health Organization adult ADHD self-report scale (ASRS): a short screening scale for use in the general population,” Psychological Medicine, 35: 245-256 (2005)). Symptoms are rated on a frequency basis: 0=never, 1=rarely, 2=sometimes, 3=often, and 4=very often. Nine items assess inattention and nine assess hyperactivity-impulsivity. The 9 inattentive symptoms are summed to create the ASRS A subscale; the 9 hyperactive-impulsive symptoms are summed to compute the ASRS B subscale. These two scales are summed to compute the total score. For all scales, higher scores indicate more symptoms. The scale has high concurrent validity with a rater-administered ADHD symptom scale. (Adler, L. A. et al., “Validity of Pilot Adult ADHD Self-Report Scale (ASRS) to Rate Adult ADHD Symptoms,” Ann. Clin. Psychiatry, 18(3):145-148 (2006)). Table 5 lists the World Health Organization (WHO) Adult ADHD Self-Report Scale (ASRS) questions.

TABLE 5
The World Health Organization (WHO) Adult ADHD Self-Report
Scale (ASRS) Questions
Table 5: The ASRS Questions

| I. | Inattention | |
|---|---|---|
| | 1. | How often do you make careless mistakes when you have to work on a |
| | | boring or difficult project? |
| | 2. | How often do you have difficulty keeping your attention when you are doing |
| | | boring or repetitive work? |
| | 3. | How often do you have difficulty concentrating on what people say to you, |
| | | even when they are speaking to you directly?* |
| | 4. | How often do you have trouble wrapping up the fine details of a project, once |
| | | the challenging parts have been done?*† |
| | 5. | How often do you have difficulty in getting things in order when you have to |
| | | do a task that requires organization?*† |
| | 6. | How often do you have a task that requires a lot of thought, how often do you |
| | | avoid or delay getting started?† |
| | 7. | How often do you misplace or have trouble finding things at home or at |
| | | work? |
| | 8. | How often are you distracted by activity or noise around you? |
| | 9. | How often do you have problems remembering appointments or obligations?*† |
| II. | Hyperactivity; Impulsivity | |
| | 1. | How often do you fidget or squirm with your hands or your feet when you |
| | | have to sit down for a long time?† |
| | 2. | How often do you leave your seat during meetings or other situations in |
| | | which you are expected to remain seated?* |
| | 3. | How often do you feel restless or fidgety? |
| | 4. | How often do you have difficulty unwinding or relaxing when you have time |
| | | to yourself? |
| | 5. | How often do you feel overly active and compelled to do things, like you |
| | | were driven by a motor?† |
| | 6. | How often do you find yourself talking too much when you are in a social |
| | | situation? |
| | 7. | When you are in a conversation, how often do you find yourself finishing |
| | | sentences of the people that you are talking to, before they can finish them |
| | | themselves?* |
| | 8. | How often do you have difficulty waiting your turn in situations when turn |
| | | taking is required? |
| | 9. | How often do you interrupt others when they are busy?* |

Response options are: never, rarely, sometimes, often, and very often. Patients are asked to answer the questions using a 6-month recall period.
*Clinically significant symptoms levels were defined for these seven questions as responses of sometimes, often and very often. For remaining 11 questions, often and very often were the clinically significant symptom levels.
†The short six-question ASRS screener.

### Pathophysiology of Systemic Lupus Erythematosus

While the cause of SLE is unknown, its pathogenesis involves cellular dysfunction of the immune system and the production of anti-nuclear auto-antibodies. SLE is characterized by overactive B cells that differentiate into autoantibody-forming cells, mainly against nuclear material. These responses are initiated, propagated, or both by activated T cells and dendritic cells, and the production of proinflammatory cytokines and chemokines Activated T cells express CD40 ligand (CD40L) and support B cells to differentiate into plasma cells through the interaction with CD40 present on the surface of B cells. (Kyttaris, V. C. et al., “Immune cells and cytokines in systemic lupus erythematosus: an update,” Curr. Opin. Rheumatol. 17: 518-522 (2005); Tenbrock, K. et al., “Altered signal transduction in SLE T cells,” Rheumatology, 46: 1525-1530 (2007)).

The pathogenesis of SLE can be differentiated into two distinct phases. (Reviewed in Sifuentes Giraldo, W. A. et al., “New therapeutic targets in systemic lupus,” Reumatol. Clin., 8(4): 201-207 (2012)). On the one hand, interactions of genetic and exogenous environmental factors lead to the production of autoantibodies that trigger a flare in autoimmunity, which is associated with an amplifying effect involving nuclear antigens and their corresponding autoantibodies through mechanisms of both innate and adaptive immunity. The second phase of SLE pathogenesis is the development of inflammation and damage to target organs.

### Inflammation

Deregulated cytokine production in SLE contributes to immune dysfunction and mediates tissue inflammation and organ damage. Inflammatory cytokines, like type I and type II interferons and interleukin-6 (IL-6), IL-1, and tumor necrosis factor-alpha (TNF-α) as well as immunomodulatory cytokines like IL-10 and TGF-β, have been identified as important players in SLE. In addition, IL-17, IL-21 and IL-2 are implicated to play a role in autoimmunity. (Reviewed in Ohl, K. et al., “Inflammatory cytokines in systemic lupus erythematosus,” Journal of Biomedicine and Biotechnology, Vol. 2011, Article ID 432595 (2011)).

Type I Interferons

IFNs are thus responsible for a self-reinforcing vicious circle that maintains and multiplies the mechanism of autoimmunity. The primary function of Type I interferons (IFNs) is to mediate the early immune response to viral infections. Viral RNA and DNA are recognized by Toll-like receptors (TLRs) and trigger IFN release of leukocytes. Although all leukocytes produce IFN, plasmacytoid dendritic cells (PDCs) that constitutively express Toll-like receptor 7 (TLR7) and Toll-like receptor 9 (TLR9) are the primary producers with the ability to release high amounts of IFN. Upon secretion, IFN binds to its heteromeric type I IFN receptor on target cells, transduces signals mainly through JAK/STAT pathways, and initiates gene transcription of interferon-stimulated genes. IFNs activate genes that are responsible for antimicrobial responses, antigen processing, and inflammation, thereby exerting several key immunomodulatory effects in both innate and adaptive immune responses.

SLE patients often have enhanced IFN-α serum levels that also correlate with anti-ds DNA production. (Reviewed in Ohl, K. et al., “Inflammatory cytokines in systemic lupus erythematosus,” Journal of Biomedicine and Biotechnology, Vol. 2011, Article ID 432595 (2011)). The hallmark of SLE is the formation of immune complexes (ICs). Causes of IC formation in SLE are an increased apoptosis and defective clearance of apoptotic material on one hand and high occurrence of autoantibodies on the other. (Reviewed in Ohl, K. et al., “Inflammatory cytokines in systemic lupus erythematosus,” Journal of Biomedicine and Biotechnology, Vol. 2011, Article ID 432595 (2011)). The secretion of autoantibodies and their binding to nuclear antigens lead to the formation of immune complexes (ICs), which primarily contain DNA/anti-DNA complexes, with a capacity to deposit in tissues where the ICs, along with participation of the complement system, produce lesions. These ICs with nuclear products are also taken up by plasmocytoid dendrtic cells (PDCs) through FCγ-IIAr receptors and engulfed into endosomes. Toll-like receptors TLR 7 and 9 anchored within the endosomal membranes are activated by the ICs triggering a transduction cascade that leads to the activation of IRF 7/5 and NFκB transcription factors that in turn drive the production of IFNα, and to a lesser extent other proinflammatory cytokines, such as IL-6. (Reviewed in Sifuentes, W. A. et al., “New therapeutic targets in systemic lupus,” Reumatol. Clin., 8(4): 201-207 (2012)).

The overproduction of IFNs in SLE exerts wide effects. Firstly, IFN-α promotes feedback loops by the induction of TLR7 in the dendritic cells and monocytes, which enhance the synthesis of IFN. Secondly, IFNs contribute to disruption of peripheral tolerance by promoting DC maturation (mDC), thereby reducing the number of immature DCs that are important for maintenance of immune tolerance and regulatory T (Treg) cells. In addition, immature DCs promote deletion of self-reactive T cells by presenting self-peptide MHC complexes in the absence of costimulatory signals to self-reactive T cells. Activated and self-reactive T cells provide help for B cells. Thirdly, mDCs can also directly enhance selection and survival of autoreactive B cells by producing B cell activating factor (BAFF). Finally, IFN-α drives disease activity by enhancing cytotoxicity of CD8+ T cells and directly increases numbers of autoreactive CD4+ T cells by upregulation of the costimulatory molecules CD80 and CD86 on antigen-presenting cells (APCs). (Reviewed in Ohl, K. et al., “Inflammatory cytokines in systemic lupus erythematosus,” Journal of Biomedicine and Biotechnology, Vol. 2011, Article ID 432595 (2011); and Sifuentes, W. A. et al., “New therapeutic targets in systemic lupus,” Reumatol. Clin., 8(4): 201-207 (2012)).

Interleukin-6

IL-6 is produced in many cell types, including but not limited to, monocytes, fibroblasts, endothelial cells, and also T and B lymphocytes and has a wide range of biological activities on various target cells. IL-6 serves as a differentiation factor for several hematopoetic cells and as a major hepatocyte stimulation factor, and is also responsible for induction of differentiation of B cells into plasma cells, induction of IgG production, differentiation and proliferation of T cells and macrophages, bone marrow stem cell maturation, activation of neutrophils, and stimulation of the production of platelets from megacaryocytes and osteoclast differentiation. 11-6 is also a key cytokine in determining the differentiation of naïve T cells into regulatory T cells with a suppressive phenotype or into T cells with a proinflammatory phenotype. (Reviewed in Ohl, K. et al., “Inflammatory cytokines in systemic lupus erythematosus,” Journal of Biomedicine and Biotechnology, Vol. 2011, Article ID 432595 (2011)).

IL-6 signaling occurs via its heteromeric receptor complex, consisting of two glycoproteins, an IL-6 specific binding chain (IL-6R) and a signal transducing chain (gp130). Binding of IL-6 on Il-6R triggers dimerization of gp130, which activates JAK1 and tyrosine phosphorylation of gp130. This in turn activates the ERK/MAPK signaling pathway and p-STAT3-mediated pathways.

Murine lupus models indicate involvement of IL-6 in B-cell hyperactivation and onset of autoimmune disease. Patients with active SLE have increased IL-6 serum levels that correlate with disease activity or anti-DNA levels. Elevated IL-6 levels are associated with B-cell hyperactivity and autoantibody production. In addition to systemic effects, IL-6 is also implicated in local inflammation; for example in lupus nephritis, patients show elevated levels of IL-6 in urine. IL-6 is increased during cardiopulmonary complications of SLE, and SLE patients with neuropsychiatric syndromes show elevated IL-6 levels in the cerebrospinal fluid. (Reviewed in Ohl, K. et al., “Inflammatory cytokines in systemic lupus erythematosus,” Journal of Biomedicine and Biotechnology, Vol. 2011, Article ID 432595 (2011)).

Interferon-Gamma

Interferon-gamma (IFN-γ) activates macrophages at the site of inflammation, contributes to cytotoxic T-cell activity, has antiviral capacities, and is strongly associated with T helper 1 (T_{H1}) responses. It induces differentiation of naïve T cells into T_{H1} cells and triggers T_{H1} differentiation. IFN-γ signaling induces phosphorylation of STAT1 which leads to expression of the Th1-lineage specific transcription factors and subsequent expression of IFN-γ. (Reviewed in Ohl, K. et al., “Inflammatory cytokines in systemic lupus erythematosus,” Journal of Biomedicine and Biotechnology, Vol. 2011, Article ID 432595 (2011)).

The role of IFN-γ in SLE has been studied in several mouse models. For example, T-helper cells expressing IFN-γ correlate with age and development of disease in NZB/W F1 mice. IFN-γ accelerated development of disease, while administration of monoclonal antibodies against IFN-γ resulted in remission of disease. (Reviewed in Ohl, K. et al., “Inflammatory cytokines in systemic lupus erythematosus,” Journal of Biomedicine and Biotechnology, Vol. 2011, Article ID 432595 (2011)).

Interleukin-2

T cells are the main producer and responder cells of interleukin-2 (IL-2). IL-2 production is induced after T-cell receptor (TCR) activation. IL-2 is a growth factor that is crucial in preventing formation of autoimmunity, and is important in maintaining functionality and homeostasis of regulatory T (Treg) cells on one hand and also in preventing overproduction of IL-17.

SLE T cells show reduced IL-2 production, and IL-2 deficiency is also paralleled by low numbers of Treg cells in SLE patients. The molecular mechanism of the IL-2 defect in SLE is caused, for example, by overexpression of cAMP response element modulator alpha (CREMα), a transcription factor which binds to the IL-2 promoter and inhibits IL-2 transcription. Defective IL-2 production in SLE T cells contributes to several immune alterations including reduced numbers and function of Treg cells, decreased activation induced cell death (AICD), which is a controlled apoptotic mechanism by which effector cells are eliminated, decreased cytotoxic T cell (CTL) responses, and upregulation of IL-17 production. (Reviewed in Ohl, K. et al., “Inflammatory cytokines in systemic lupus erythematosus,” Journal of Biomedicine and Biotechnology, Vol. 2011, Article ID 432595 (2011)).

Interleukin-21

IL-21 is produced by a range of differentiated CD4+ T cell subsets and natural killer (NK) T cells. IL-21 signals through a heterodimeric receptor, which is formed by common gamma chain (shared with IL-2, IL-4, IL-7, IL-9, IL-13 and IL-15 receptors) and an IL-21 specific receptor (IL-21R). Since IL-21 is expressed on CD4+, CD8+, T cells, B cells, NK cells, dendritic cells, macrophages, and keratinocytes, IL-21 acts on a range of lymphoid lineages and exerts pleiotropic effects. IL-21 is a stimulator of CD8+ T cell proliferation. In synergy with IL-15 and IL-7, it promotes CD8+ T cell expansion; it drives differentiation of naïve T cells into TH17 cells. Induced Treg cells are negatively regulated by IL-21 and IL-21 in turn counteracts the suppressive effects of Treg cells.

SLE patients have higher serum IL-21, while IL-21 and IL-21R polymorphisms are associated with susceptibility to SLE. (Reviewed in Ohl, K. et al., “Inflammatory cytokines in systemic lupus erythematosus,” Journal of Biomedicine and Biotechnology, Vol. 2011, Article ID 432595 (2011)).

Interleukin-17

IL-17 is produced by several T-cell subsets including T helper cells (CD4+ T cells), cytotoxic T cells (CD8+ T cells), double negative (CD4− CD8− CD3+) T cells, gamma-delta T cells, natural killer (NK) cells and neutrophils. IL-17 exerts many effects on diverse cell types. For example, on T cells, IL-17 induces production of proinflammatory IL-6, IL-1beta, and IL-21 providing a feedback loop, and enhances recruitment of T cells to inflamed tissue. In B cells, IL-17 drives B-cell differentiation into plasma cells and production of autoantibodies. IL-17 receptors are broadly expressed not only on immune cells, but also on epithelial and endothelial cells. IL-17 signaling through its receptors increases production of chemokines (interleukin-8 (IL-8), monocytes chemoattractant protein-1, growth-related oncogene protein-alpha), which leads to recruitment of monocytes and neutrophils into inflamed tissue. IL-17 induces T-cell infiltration by upregulating the expression of intercellular adhesion molecule 1 (ICAM1). IL-17 induces secretion of many proinflammatory proteins, such as prostaglandin E2, granulocyte-macrophage colony stimulating factor (GM-CSF), and granulocyte colony stimulating factor, and also cytokines which induce a positive feedback loop and lead to further production of IL-17, IL-6, IL-1β and IL-21.

SLE patients have raised serum levels of IL-17. Enhanced percentages of IL-17 producing cells and plasma IL-17 levels correlate with disease activity. One source of IL-17 in SLE patients are the double negative T cells (DNs). SLE patients have expanded numbers of DNTs compared to healthy individuals. IL-17 producing cells infiltrate skin, lung, and kidneys of SLE and lupus nephritis patients.

### T-Cell Cycle Dysfunction in SLE

Abnormal T cell activation and cell death are hallmarks of SLE pathology. Potentially autoreactive T and B lymphocytes are removed by apoptosis during development and after completion of an immune response. However, SLE T cells exhibit both enhanced spontaneous apoptosis and defective activation-induced cell death (AICD). (Fernandez, D. and Perl, A., “Metabolic control of T-cell activation and death in SLE,” Autoimmun. Rev., 8(3): 184-189 (2009)).

Activation of the Mammalian Target of Rapamycin (mTOR)

The mammalian target of rapamycin (mTOR) is located in the outer mitochondrial membrane and serves as a sensor of the Δψm in T cells. mTOR is a key eukaryotic signaling protein conserved from yeast to humans, which regulates protein synthesis and energy expenditure. It acts as a central junction that integrates many inputs relaying information about nutritional status of the cell, including mitochondrial potential, oxygen tension, growth signals, amino acids, and ATP. In conditions of nutrient sufficiency, mTOR signaling is active, permitting protein synthesis and increased cell size. In nutrient deficient conditions, mTOR activity decreases, limiting energy expenditure by inhibiting protein synthesis, decreasing cell size, and preventing cell proliferation. (Fernandez, D. and Perl, A., “Metabolic control of T-cell activation and death in SLE,” Autoimmun. Rev., 8(3): 184-189 (2009); Fernandez, D. and Perl, A., “mTOR Signaling: a central pathway to pathogenesis in systemic lupus erythematosus,” Discov. Med., 9(46): 173-178 (2010); Laplante, M. and Sabatini, D. M., “mTOR signaling at a glance,” J. Cell Sci., 122: 3589-3594 (2009)).

mTOR skews cell death signal processing, modulates T-cell differentiation, and, in particular, inhibits the development of CD4⁺/CD25⁺/Foxp3⁺ regulatory T cells, which are deficient in patients with active SLE (Fernandez, D. R. and Perl, A., “mTOR signaling: a central pathway to pathogenesis in systemic lupus erythematosus?” Discov. Med. 9: 173-178 (2010); Fernandez, D. and Perl, A., “Metabolic control of T cell activation and death in SLE,” Autoimmun. Rev. 8:184-189 (2009); Battaglia, M. et al., “Rapamycin Promotes Expansion of Functional CD4+CD25+FOXP3+Regulatory T Cells of Both Healthy Subjects and Type 1 Diabetic Patients,” J. Immunol. 177: 8338-8347 (2006); Crispin, J. C. et al., “Quantification of regulatory T cells in patients with systemic lupus erythematosus,” J. Autoimmun., 21: 273-276 (2003); Valencia, X. et al., “Deficient CD4+CD25high T Regulatory Cell Function in Patients with Active Systemic Lupus Erythematosus,” J. Immunol., 178: 2579-2788 (2007)).

Although mTOR is highly conserved and controls protein translation and other metabolic pathways in all mammalian cells, it plays a critical role in T cell activation Inhibition of mTOR by rapamycin blocks T cell function. mTOR activity is increased in lupus T cells. Activation of mTOR is inducible by NO, a key trigger for MHP and mitochondrial biogenesis. In turn, NO-induced stimulation of HRES-1/Rab4 is reduced by rapamycin. Thus, NO-dependent MHP lies upstream, whereas enhanced expression of HRES-1/Rab4 lies downstream of mTOR activation in lupus T cells. Further downstream, CD4, Lck, and TCRζ protein levels are depleted, whereas Syk and FcεRIγ levels are augmented in lupus T cells, all of which can be reversed in SLE patients treated with rapamycin in vivo. Depletion of TCRζ in lupus T cells is reversed by HRES-1/Rab4 knockdown as well as by inhibition of lysosomal function in vitro, indicating that activation of mTOR causes the loss of TCRζ through HRES-1/Rab4-dependent lysosomal degradation. (Fernandez, D. R. et al., “Activation of mammalian target of rapamycin controls the loss of TCRζ in Lupus T cells through HRES-1/Rab4-regulated lysosomal degradation,” J. Immunol., 182: 2063-2073 (2009)).

Blockade of mTOR with rapamycin, a potent and expensive immunosuppressant, improves disease activity in murine lupus. Rapamycin normalized T-cell mitogen-stimulated splenocyte proliferation and IL-2 production, prevent increase in anti-double-stranded DNA antibody and urinary albumin levels and glomerulonephritis (GN), and prolonged survival of lupusprone MRL/lpr lupus mouse model. (Warner, L. M. et al., “Rapamycin prolongs survival and arrests pathophysiologic changes in murine systemic lupus erythematosus,” Arth. Rheum. 37: 289-297 (1994)). Treatment of human SLE patients resistant or intolerant to conventional medications with rapamycin improves disease activity. Treatment of rapamycin is also associated with normalization of baseline Ca²⁺ levels in the cytosol and mitochondria and of CD3/CD28-induced Ca²⁺ fluxing with no effect on mitochondrial potential, which remained elevated in both the treated and control groups. This observation indicated that increased Ca²⁺ fluxing is downstream or independent of MHP in the pathogenesis of T cell dysfunction in SLE. (Fernandez, D. et al., “Rapamycin reduces disease activity and normalizes T-cell activation-induced calcium fluxing in patients with systemic lupus erythematosus,” Arth. Rheum. 54(9): 2983-2988 (2006)).

Oxidative Stress

Both cell proliferation and apoptosis are energy dependent processes. Energy in the form of ATP is available through glycolysis and oxidative phosphorylation. The site of oxidative phosphorylation is the mitochondria. Each mitochondrion is bounded by two specialized membranes (i.e., the inner and outer mitochondrial membranes) that create two separate mitochondrial compartments: the internal matrix and the intermembrane space. ATP synthesis is driven by an electrochemical gradient across the inner mitochondrial membrane maintained by an electron transport chain. The transfer of electrons is coupled to proton (H+) uptake and release and allosteric changes in energy-converting transmembrane protein pumps. The net result is the pumping of H+ across the inner mitochondrial membrane from the matrix to the intermembrane space, driven by the energetically favorable flow of electrons. This movement of H+ has two consequences: (1) the generation of a pH gradient across the inner mitochondrial membrane, with the pH higher in the matrix than in the cytosol, where the pH is generally close to 7; and (2) generation of a membrane potential (Δψₘ) across the inner mitochondrial membrane, with the inside negative and outside positive as a result of the net outflow of positive ions. (“Chapter 14: Energy conversion: mitochondria and chloroplasts,” Molecular Biology of the Cell, Alberts, B. et al., Garland Science, N.Y., 2002, pp. 775; Fernandez, D. and Perl, A., “Metabolic control of T-cell activation and death in SLE,” Autoimmun. Rev., 8(3): 184-189 (2009)).

Mitochondrial hyperpolarization (MHP) refers to the generation of the mitochondrial membrane potential (Δψₘ)). It is the result of an electrochemical gradient maintained by two transport systems—the electron transport chain and the F₀F₁-ATPase complex. The electron transport chain catalyzes the flow of electrons from NADH to molecular oxygen and the translocation of protons across the inner mitochondrial membrane, thus creating a voltage gradient with negative charges inside the mitochondrial matrix. A small fraction of electrons react directly with oxygen and form reactive oxygen intermediates (ROIs) Innate and adaptive immune responses depend on the controlled production of ATP and ROIs in the mitochondria. (Perl, A. et al., “Mitochondrial hyperpolarization: a checkpoint of T-cell life, death and autoimmunity,” Trends in Immunology, 25(7): 360-367 (2004)).

With MHP and extrusion of H+ ions from the mitochondrial matrix, the cytochromes within the electron transport chain become more reduced, which elevates ROI production and generates oxidative stress. ROIs modulate various aspects of T cell activation, cytokine production, and apoptosis. (Perl, A. et al., “Mitochondrial hyperpolarization: a checkpoint of T-cell life, death and autoimmunity,” Trends in Immunology, 25(7): 360-367 (2004); Perl, A., “Systems Biology of lupus: mapping the impact of genomic and environmental factors on gene expression signatures, cellular signaling, metabolic pathways, hormonal and cytokine imbalance, and selecting targets for treatment,” Autoimmunity, 43(1): 32-47 (2010)).

Regulation of the mitochondrial membrane potential (Δψₘ) is an important checkpoint in determining T cell fate. Mitochondrial hyperpolarization (MHP) is an early event of T-cell activation and death that is mediated through inhibition of F₀F₁-ATPase or dephosphorylation of cytochrome c oxidase. Nitric oxide (NO), acting as a competitive antagonist of oxygen, can also reversibly inhibit cytochrome c oxidase and cause MHP. Using the energy of ATP, F₀F₁-ATPase can pump protons out of the mitochondrial matrix into the intermembrane space, thus causing Δψₘ elevation. MHP leads to uncoupling of oxidative phosphorylation (i.e. continued and enhanced ROI production in the absence of ATP synthesis), which disrupts Δψₘ and damages the integrity of the inner mitochondrial membrane. (Perl, A. et al., “Mitochondrial hyperpolarization: a checkpoint of T-cell life, death and autoimmunity,” Trends in Immunology, 25(7): 360-367 (2004); Fernandez, D. and Perl, A., “Metabolic control of T-cell activation and death in SLE,” Autoimmun. Rev., 8(3): 184-189 (2009)).

The antigen-binding αβ or γδ T-cell receptor (TCR) is associated with a multimeric receptor module comprising the CD3 γδε and ζ chains. The cytoplasmic domain of CD3ζ chain contains an immunoglobulin receptor family tyrosine-based activation motif (ITAM) which is crucial for coupling of intracellular tyrosine kinases. Expression of CD3ζ is suppressed by ROIs. Binding of p56lck (a lymphocyte-specific protein tyrosine kinase) to CD4 or CD8 attracts this kinase to the TCR-CD3 complex, leading to phosphorylation of ITAM. Phosphorylation of both tyrosines of each ITAM is required for SH-2-mediated binding by zeta-associated protein-70 (ZAP-70) or the related SYK. ZAP-70 is activated through phosphorylation by p56lck. Activated ZAP-70 and SYK target two key adaptor proteins, LAT and SLP-76. (Perl, A., “Systems Biology of lupus: mapping the impact of genomic and environmental factors on gene expression signatures, cellular signaling, metabolic pathways, hormonal and cytokine imbalance, and selecting targets for treatment,” Autoimmunity, 43(1): 32-47 (2010)).

Phosphorylated LAT binds directly to phospholipase C-γ1 (PLC γ1) that controls hydrolysis of phosphatidylinositol-4,5-biphosphate (PIP2) to inositol-1,4,5-triphosphate (IP3) and diacylglycerol (DAG). Phosphorylation of inositol lipid second messengers is mediated by phosphatidylinositol 3′ hydroxyl kinase (PI3K). IP3 binds to its receptors in the endoplasmic reticulum (ER), opening Ca²⁺ channels that release Ca²⁺ to the cytosol. Decreased ER Ca²⁺ concentration activates the Ca²⁺ release-activated Ca²⁺ channel (CRAC) in the cell membrane. The resultant Ca²⁺ influx activates the phosphatase calcineurin, which dephosphorylates a transcription factor called nuclear factor of activated T cells (NFAT). Dephosphorylated NFAT can translocate to the nucleus where it promotes transcription of IL-2 and NF-κB. Lupus T cells have decreased amounts of DNA-binding 98 kDa form of the Elf-1 transcription factor that reduces the expression of TCRζ. Lupus T cells exhibit persistent MHP and ATP depletion, which causes predisposition to death by necrosis that is highly proinflammatory. (Perl, A. et al., “Mitochondrial hyperpolarization: a checkpoint of T-cell life, death and autoimmunity,” Trends in Immunology, 25(7): 360-367 (2004); Fernandez, D. and Perl, A., “Metabolic control of T-cell activation and death in SLE,” Autoimmun. Rev., 8(3): 184-189 (2009); (Perl, A., “Systems Biology of lupus: mapping the impact of genomic and environmental factors on gene expression signatures, cellular signaling, metabolic pathways, hormonal and cytokine imbalance, and selecting targets for treatment,” Autoimmunity, 43(1): 32-47 (2010)).

Nitric Oxide (NO) Exposure

NO provides a link between T cell activation and mitochondrial function. NO is produced by nitric oxide synthases (NOS) that requires Ca²⁺ to function and use NADPH and arginine as substrates. Three isoforms of NOS exist: endothelial NOS (eNOS), neuronal NOS (nNOS), and inducible NOS (iNOS), of which T cells express the former two. NO induces MHP and mitochondrial biogenesis, increases Ca²⁺ in the cytosol and mitochondria of normal T cells, and recapitulates the enhanced CD3/CD28-induced Ca²⁺ fluxing of lupus T cells. (Fernandez, D. and Perl, A., “Metabolic control of T-cell activation and death in SLE,” Autoimmun. Rev., 8(3): 184-189 (2009)).

NO induces (1) the expression of HRES-1/Rab4 (a small GTPase encoded in the HRES-1 human endogenous retrovirus genome) that regulates receptor recycling and targets CD4 and TCRζ for lysosomal degradation; and (2) promotes mitochondrial biogenesis and mitochondrial storage of Ca²⁺. (Perl, A., “Systems Biology of lupus: mapping the impact of genomic and environmental factors on gene expression signatures, cellular signaling, metabolic pathways, hormonal and cytokine imbalance, and selecting targets for treatment,” Autoimmunity, 43(1): 32-47 (2010)).

Endothelial NOS is recruited to the site of T-cell receptor engagement, locally increasing NO at the immunological synapse in a Ca²⁺ and PI3K-dependent manner, resulting in reduced IL-2 production which is characteristic of SLE. (Fernandez, D. and Perl, A., “Metabolic control of T-cell activation and death in SLE,” Autoimmun. Rev., 8(3): 184-189 (2009)).

Depletion of Intracellular Glutathione (GSH)

Mitochondrial membrane integrity is dependent on the oxidation-reduction equilibrium of ROIs, pyridine nucleotides (NADH/NAD and NADPH/NADP) and reduced glutathione (GSH) levels. GSH is a small ubiquitous cysteine-containing tripeptide (γ-glutamylcysteinylglycine) found in millimolar concentrations in animal cells, and provides the principal intracellular defense against oxidative stress and participates in detoxication of many molecules.

A normal reducing atmosphere, required for cellular integrity, is provided by reduced GSH, which protects cells from damage by ROIs. Synthesis of GSH from its oxidized form, GSSG, is completely dependent on NADPH produced by the pentose phosphate pathway (PPP). A fundamental function of PPP is to maintain glutathione in a reduced state and thus provide protection of sulfhydryl groups and cellular integrity from emerging oxygen radicals. PPP comprises two separate but functionally connected branches: the oxidative and the nonoxidative branches. Reactions in the oxidative branch are irreversible, whereas all reactions of the nonoxidative branch are fully reversible. The nonoxidative branch can convert ribose 5-phosphate into glucose 6-phosphate for the oxidative branch, and thus, indirectly, it can also contribute to generation of NADPH. The rate-limiting enzymes for the two branches are different. The oxidative phase is primarily dependent on glucose-6-phosphate dehydrogensae (G6PD). While the control of the nonoxidative branch is less well established, transaldolase (TAL) has been proposed as its rate-limiting enzyme. TAL catalyzes the transfer of a 3-carbon fragment, corresponding to dihydroxyacetone, to D-glyceraldehyde 3-phosphate, D-erythrose 4-phosphate, and a variety of other acceptor aldehydes, including nonphosphorylated trioses and tetroses. (Banki, K. et al., “Glutathione levels and sensitivity to apoptosis are regulated by changes in transaldolase expression,” J. Biol. Chem., 271(51): 32994-33001 (1996)). ROI levels and the mitochondrial membrane potential (Δψₘ) are regulated by transaldolase through the supply of reducing equivalents from PPP, Ca²⁺ fluxing and nitric oxide (NO) production.

Reduced GSH constitutes the majority of the intracellular glutathione in the body. Oxidized GSH (GSSG) is formed during normal oxidative metabolism. It is also produced when cells are subjected to oxidative stress or to exogenous toxins that are detoxified by conjugation to GSH. GSSG and GSH conjugates are released rapidly from cells and excreted from the body; relatively little GSSG is recycled to GSH. GSH levels commonly are measured by HPLC or mass spectrometry as total GSH reduced and extracted from circulating erythrocytes (or separately as erythrocyte GSH and GSSG). Stores of reduced GSH are influenced greatly by nutritional status, presence of certain disease states, and exposures to oxidative stressors and molecules that are detoxified by conjugation with GSH.

Viral, bacterial, and fungal infections, malnutrition, chronic and acute alcohol consumption, diabetes, certain metabolic diseases, and consumption of oxidative drugs all have been shown to decrease GSH. GSH deficiency has been demonstrated in many diseases including but not limited to hepatic conditions (e.g. acetaminophen toxicity, alcohol, hepatitis, liver disease, liver transplantation), renal conditions (e.g., chronic kidney failure, dialysis, alpha-amanintin), cardiovascular disorders (e.g., angina, arteriosclerosis/cardiac risk, myocardial infarction, cardiomyopathy), endocrine disorders (e.g., diabetes), pulmonary disorders (e.g., bronchopulmonary disorders, fibrosing alveolitis, chronic asthma, chronic bronchitis, acute respiratory distress syndrome (ARDS), chronic obstructive pulmonary disease (COPD), cystic fibrosis, pulmonary fibrosis, smoking, lung cancer), conditions encounted in critical care (e.g., sepsis, septic shock, malnutrition), infections (e.g., human immunodeficiency virus (HIV), Helicobacter pylori, influenza, malaria, epilepsy), gastrointestinal disorders (e.g., inflammatory bowel disease, Barrett's esophagus), optic conditions (e.g., blepharitis, cataract, Eale's disease), skin conditions (e.g., psoriasis, photodermatitis), immune conditions (e.g., rheumatoid arthritis, autoimmune disorders), urogenital disorders, muscular conditions (e.g., exercise), aging, toxic agents (e.g., arsenic poisoning), perinatal conditions (e.g., preeclampsia, neonates), and metabolism (e.g., phenylketonuria). GSH is essential to cell survival. It plays key roles in cellular metabolism and protection against oxidative and other toxic molecules, including those generated in response to attack by cytokines that induce pain and fever.

The natural antioxidant glutathione (GSH) is also depleted in the peripheral blood leukocytes (PBLs) and lymphocytes of patients with SLE. Low GSH in T cells over-expressing transaldolase predispose to mitochondrial hyperpolarization (MHP). The effect of nitric oxide (NO) on MHP is tightly related to GSH levels. GSH depletion triggers MHP upon exposure to NO. Diminished production of GSH in face of MHP and increased ROI production is suggestive of a metabolic defect in de novo GSH synthesis or maintenance of its reduced state due to deficiency of NADPH. (Fernandez, D. and Perl, A., “Metabolic control of T-cell activation and death in SLE,” Autoimmun. Rev., 8(3): 184-189 (2009); Shah, D. et al., “Association between T lymphocyte sub-sets apoptosis and peripheral blood mononuclear cells oxidative stress in systemic lupus erythematosus,” Free Rad. Res., 45: 559-567 (2011)). GSH regulates the elevation of mitochondrial transmembrane potential (Δψₘ) or mitochondrial hyperpolarization (MHP), which in turn activates the mammalian target of rapamycin (mTOR) in lupus T cells (Fernandez, D. R. et al., “Activation of mTOR controls the loss of TCRζ in lupus T cells through HRES-1/Rab4-regulated lysosomal degradation,” J. Immunol. 182: 2063-2073 (2009)).

### N-Acetylcysteine (NAC)

N-acetylcysteine (NAC), which serves as a precursor of glutathione (GSH) and an antioxidant in and of itself, inhibits mTOR in vitro and improves the outcome of murine lupus in vivo. (O'Loghlen, A. et al., “N-acetyl-cysteine abolishes hydrogen peroxide-induced modification of eukaryotic initiation factor 4F activity via distinct signalling pathways,” Cell. Signal 18: 21-31 (2006); Suwannaroj, S. et al., “Antioxidants suppress mortality in the female NZB×NZW F1 mouse model of systemic lupus erythematosus (SLE),” Lupus, 10: 258-265 (2001)). GSH is a tripeptide composed of cysteine, glutamic acid, and glycine. The availability of cysteine is rate-limiting for GSH synthesis. (Wernerman, J. and Hammarqvist, F., “Modulation of endogenous glutathione availability,” Curr. Opin. Clin. Nutr. Metab. Care 2:487-92 (1999)).

NAC, the N-acetylated form of L-cysteine (also known as acetylcysteine, mercapturic acid) is a direct antioxidant (electron donor), biochemical precursor and stable transport form of cysteine and efficient prodrug for glutathione (GSH). It has the advantages of resistance to oxidation and permeability through cell membrane over other forms of cysteine supplementation. (Wernerman, J. and Hammarqvist, F., “Modulation of endogenous glutathione availability,” Curr. Opin. Clin. Nutr. Metab. Care 2:487-92 (1999)). It can effectively raise intracellular GSH of lymphocytes both in vitro and in vivo (Banki, K. et al., “Glutathione Levels and Sensitivity to Apoptosis Are Regulated by changes in Transaldolase expression,” J. Biol. Chem., 271: 32994-33001 (1996); Herzenberg, L. A. et al., “Glutathione deficiency is associated with impaired survival in HIV disease,” Proc. Natl. Acad. Sci. U.S.A. 94: 1967-1972 (1997)).

In a European study of idiopathic pulmonary fibrosis patients, “high-dose” oral NAC (1.8 g/day) diminished disease severity and reduced the toxicity of pro-oxidant and immunosuppressant medications commonly used in patients with SLE ((Demedts, M. et al., “High-dose acetylcysteine in idiopathic pulmonary fibrosis,” N. Engl. J. Med., 353: 2229-2242 (2005); Francis, L. and Perl, A., “Pharmacotherapy of systemic lupus erythematosus,” Expert Opin. Pharmacother., 10: 1481-1494 (2009)). Similar doses of NAC were reported to improve muscle fatigue which is reported to be the most disabling symptom in 53% of SLE patients. (Travaline, J. M. et al., “Effect of N-acetylcysteine on human diaphragm strength and fatigability,” Am. J. Resp. Crit. Care Med., 156: 1567-1571 (1997); Krupp, L. B. et al., “A study of fatigue in systemic lupus erythematosus,” J. Rheumatol., 17: 1450-1452 (1990)). NAC has also been reported to improve memory and cognitive function (Martinez, M. et al., “N-Acetylcysteine delays age-associated memory impairment in mice: Role in synaptic mitochondria,” Brain Res. 855(1): 100-106 (2000)), bipolar disease (Bernardo, M. et al., “Effects of N-acetylcysteine on substance use in bipolar disorder: A randomised placebo-controlled clinical trial,” Acta Neuropsych. 21(5): 239-245 (2009)), and schizophrenia in controlled clinical trials (Berk, M. et al., “N-Acetyl Cysteine as a Glutathione Precursor for Schizophrenia-A Double-Blind, Randomized, Placebo-Controlled Trial,” Biol. Psych., 64(5): 361-368 (2008)).

### Treatment of SLE

The current treatment of SLE includes nonsteroidal anti-inflammatory drugs, antimalarial agents, corticosteroids, high dose immunoglobulins, and cytotoxic immunosuppressive agents such as aspirin, azathioprine, cyclophosphamide, methotrexate, and mycophenolic acid. These treatments, while effective, are nonspecific and can have an indiscriminate immunosuppressive effect which often leads to severe adverse events and opportunistic infections. Many of these drugs are not approved for SLE treatment. In spite of this, the use of these off label drugs, along with improved management of common symptoms, such as hypertension, dyslipidemia, nephrotic syndrome, etc. has improved its long term prognosis.

Many biological immune-targeted treatments are currently been developed for the treatment of SLE. These include, but are not limited to, B-cell targeted therapy, cytokine blockade, peptide-based treatments, etc. (Bezalel, S. et al., “Novel biological treatments for systemic lupus erythematosus: current and future modalities,” Israeli Medical Association Journal, 14: 508-514 (2012); Sifuentes Giraldo, W. A. et al., “New therapeutic targets in systemic lupus,” Rheumatologia Clinica, 8(4): 201-207 (2012)). However, because of the serious adverse effects that can worsen life expectancy and quality of life, novel immune targeted treatments for SLE has become a universally recognized need.

The described invention provides an immune-targeted treatment of SLE using compositions comprising N-acetylcysteine (NAC). The described composition is safe, well-tolerated and efficacious pharmaceutical composition comprising a therapeutic amount of NAC for this use. The described pharmaceutical composition and method of using the composition provide clinically significant improvement of at least one lupus disease activity index (e.g., BILAG, SLEDAI, or both) within at least 3 months of treatment; diminished fatigue; and absence of significant side effects.

### SUMMARY

According to one aspect, the described invention provides a method of treating a lupus condition in a subject in need thereof, comprising: (a) providing a pharmaceutical composition comprising a therapeutic amount of a compound N-acetyl-L-cysteine (NAC) of Formula I:

[chemical structure]

or a pharmaceutically acceptable salt, solvate, prodrug, or a derivative thereof; and a pharmaceutically acceptable carrier; and (b) administering the pharmaceutical composition to the subject, wherein the therapeutic amount is effective to decrease activity of mammalian target of rapamycin (mTOR) and to treat one or more symptoms of the lupus condition.

According to one embodiment, the lupus condition is systemic lupus erythematosus (SLE). According to another embodiment, the therapeutic amount of N-acetyl-L-cysteine (NAC) for an adult is a maximum daily dose of about 4800 mg/day to about 8000 mg/day. According to another embodiment, the therapeutic amount of the N-acetyl-L-cysteine (NAC) is effective to reduce lupus disease activity in the subject compared to an untreated control. According to another embodiment, the lupus disease activity is measured by a disease activity score selected from the group consisting of systemic lupus erythematosus disease activity index (SLEDAI) score, British Isles Lupus Assessment Group (BILAG) score, fatigue assessment scale (FAS) score, or a combination thereof. According to another embodiment, the systemic lupus erythematosus disease activity index (SLEDAI) score of the subject is reduced compared to an untreated control at least after at least 7 days of the administration, after at least 14 days of the administration, after at least 1 month of the administration, after at least 2 months of the administration, after at least 3 months of the administration, or after at least 4 months of the administration. According to another embodiment, the systemic lupus erythematosus disease activity index (SLEDAI) score of the subject is reduced by at least 1 point to at least 2.3 points compared to an untreated control after at least 1 month of the administration. According to another embodiment, the British Isles Lupus Assessment Group (BILAG) score of the subject is reduced compared to an untreated control at least after at least 7 days of the administration, after at least 14 days of the administration, after at least 1 month of the administration, after at least 2 months of the administration, after at least 3 months of the administration, or after at least 4 months of the administration. According to another embodiment, the British Isles Lupus Assessment Group (BILAG) score of the subject is reduced by at least 1.0 point to at least 5.0 points compared to an untreated control after at least 1 month of the administration. According to another embodiment, the fatigue assessment scale (FAS) score of the subject is reduced compared to an untreated control at least after at least 7 days of the administration, after at least 14 days of the administration, after at least 1 month of the administration, after at least 2 months of the administration, after at least 3 months of the administration, or after at least 4 months of the administration. According to another embodiment, the fatigue assessment scale (FAS) score of the subject is reduced by at least 1.0 point to at least 5.0 points compared to an untreated control after at least 1 month of the administration. According to another embodiment, the therapeutic amount of the N-acetyl-L-cysteine (NAC) is effective to increase activation-induced apoptotic rate of double negative (DN) T cells of the subject compared to an untreated control after at least 7 days of the administration, after at least 14 days of the administration, after at least 1 month of the administration, after at least 2 months of the administration, after at least 3 months of the administration, or after at least 4 months of the administration. According to another embodiment, the therapeutic amount of the N-acetyl-L-cysteine (NAC) is effective to decrease activity of mammalian target of rapamycin (mTOR) compared to an untreated control after at least 7 days of the administration, after at least 14 days of the administration, after at least 1 month of the administration, after at least 2 months of the administration, after at least 3 months of the administration, or after at least 4 months of the administration. According to another embodiment, the therapeutic amount of the N-acetyl-L-cysteine (NAC) is effective to increase number of FoxP3+CD8+CD25+ T cells compared to an untreated control after at least 7 days of the administration, after at least 14 days of the administration, after at least 1 month of the administration, after at least 2 months of the administration, after at least 3 months of the administration, or after at least 4 months of the administration. According to another embodiment, the therapeutic amount of the N-acetyl-L-cysteine (NAC) is effective to reduce a cognitive/inattentive component of attention deficit and hyperactivity (ADHD) self-report scale (ASRS) compared to an untreated control after at least 7 days of the administration, after at least 14 days of the administration, after at least 1 month of the administration, after at least 2 months of the administration, after at least 3 months of the administration, or after at least 4 months of the administration.

According to another embodiment, the pharmaceutical composition further comprises at least one additional therapeutic agent selected from the group consisting of a non-steroidal anti-inflammatory agent, an antimalarial agent, a corticosteroid, a cytotoxic agent, an immunosuppressive agent, a biologic, or a combination thereof. According to another embodiment, wherein the non-steroidal anti-inflammatory agent is selected from the group consisting of aspirin, arthopan, celecoxib, diclofenac, etodolac, fenprofen, flurbiprofen, ibuprofen, ketoprofen, meclofamate, meloxicam, nabumetone, naproxen, oxaprozin, piroxicam, rofecoxib, sulindac, tolmetin, acetaminophen, or a combination thereof. According to another embodiment, the antimalarial agent is selected from the group consisting of hydroxycloroquine, chloroquine, quinicrine, or a combination thereof. According to another embodiment, the corticosteroid is in the form of a topical cream or ointment, a tablet, or an intravenous formulation. According to another embodiment, the topical cream is selected from the group consisting of clobetasol, halobetasol, hydrocortisone, triamcinolone, betamethasone, fluocinolone, fluocinonide, or a combination thereof. According to another embodiment, the tablet is selected from the group consisting of prednisone, prednisolone, ethylprednisone, or a combination thereof. According to another embodiment, the intravenous formulation is selected from the group consisting of methylprednisone, hydrocortisone, or a combination thereof. According to another embodiment, the cytotoxic agent is selected from the group consisting of azathioprine, cyclophosphamide, mycophenolate mofetil, cyclosporine A, methotrexate, chlorambucil, or a combination thereof. According to another embodiment, the immunosuppressive agent is selected from the group consisting of azathioprine, cyclophosphamide, mycophenolate mofetil, cyclosporine A, methotrexate, chlorambucil, or a combination thereof. According to another embodiment, the biologic is selected from the group consisting of a B-cell target biologic, a T cell target biologic, a spleen tyrosine kinase antagonist, a tumor necrosis factor (TNF) antagonist, an interferon antagonist, an interleukin-6-receptor antagonist, or a combination thereof

According to another embodiment, the administering in step (b) is orally, topically, parenterally, buccally, sublingually, by inhalation, or rectally. According to another embodiment, the administering in step (b) is orally. According to another embodiment, the pharmaceutical composition is in form of a tablet, a pill, a gel, a troche, a lozenge, an aqueous suspension, an oily suspension, a capsule, or a syrup. According to another embodiment, the administering in step (b) is topically. According to another embodiment, the pharmaceutical composition is in the form of an aqueous suspension, an oily suspension, an emulsion, a cream, or a patch. According to another embodiment, the administering in step (b) is parenterally. According to another embodiment, the pharmaceutical composition is in the form of an injectable solution, a gel, an aqueous suspension, an oily suspension, a granule, a bead, an emulsion, or an implant. According to another embodiment, the administering in step (b) is buccally. According to another embodiment, the pharmaceutical composition is in the form of a tablet, a pill, a gel, a troche, a lozenge, an aqueous suspension, an oily suspension, a capsule, or a syrup. According to another embodiment, the administering in step (b) is sublingually. According to another embodiment, the pharmaceutical composition is in the form of a tablet, a pill, a gel, a troche, a lozenge, an aqueous suspension, an oily suspension, a capsule, or a syrup. According to another embodiment, the administering step (b) is rectally. According to another embodiment, the pharmaceutical composition is in the form of a suppository or an insert.

According to another aspect, the described invention provides a kit for treating a lupus condition in a subject in need thereof, comprising: (a) a first packaging material containing a pharmaceutical composition comprising a therapeutic amount of a compound N-acetyl-L-cysteine (NAC) of Formula I:

[chemical structure]

or a pharmaceutically acceptable salt, solvate, prodrug, or a derivative thereof; and a pharmaceutically acceptable carrier; and (b) a means for administering the composition.

According to one embodiment, the lupus condition is systemic lupus erythematosus (SLE). According to another embodiment, the therapeutic amount of N-acetyl-L-cysteine (NAC) in the kit for an adult is a maximum daily dose of about 4800 mg/day to about 8000 mg/day. According to another embodiment, the therapeutic amount of the N-acetyl-L-cysteine (NAC) is effective to reduce lupus disease activity in the subject compared to an untreated control. According to another embodiment, the lupus disease activity is measured by a disease activity score selected from the group consisting of systemic lupus erythematosus disease activity index (SLEDAI) score, British Isles Lupus Assessment Group (BILAG) score, fatigue assessment scale (FAS) score, or a combination thereof. According to another embodiment, the systemic lupus erythematosus disease activity index (SLEDAI) score of the subject is reduced compared to an untreated control at least after at least 7 days of the administration, after at least 14 days of the administration, after at least 1 month of the administration, after at least 2 months of the administration, after at least 3 months of the administration, or after at least 4 months of the administration. According to another embodiment, the systemic lupus erythematosus disease activity index (SLEDAI) score of the subject is reduced by at least 1 point to at least 2.3 points compared to an untreated control after at least 1 month of the administration. According to another embodiment, the British Isles Lupus Assessment Group (BILAG) score of the subject is reduced compared to an untreated control at least after at least 7 days of the administration, after at least 14 days of the administration, after at least 1 month of the administration, after at least 2 months of the administration, after at least 3 months of the administration, or after at least 4 months of the administration. According to another embodiment, the British Isles Lupus Assessment Group (BILAG) score of the subject is reduced by at least 1.0 point to at least 5.0 points compared to an untreated control after at least 1 month of the administration. According to another embodiment, the fatigue assessment scale (FAS) score of the subject is reduced compared to an untreated control at least after at least 7 days of the administration, after at least 14 days of the administration, after at least 1 month of the administration, after at least 2 months of the administration, after at least 3 months of the administration, or after at least 4 months of the administration. According to another embodiment, the fatigue assessment scale (FAS) score of the subject is reduced by at least 1.0 point to at least 5.0 points compared to an untreated control after at least 1 month of the administration. According to another embodiment, the therapeutic amount of the N-acetyl-L-cysteine (NAC) is effective to increase activation-induced apoptotic rate of double negative (DN) T cells of the subject compared to an untreated control after at least 7 days of the administration, after at least 14 days of the administration, after at least 1 month of the administration, after at least 2 months of the administration, after at least 3 months of the administration, or after at least 4 months of the administration. According to another embodiment, the therapeutic amount of the N-acetyl-L-cysteine (NAC) is effective to decrease activity of mammalian target of rapamycin (mTOR) compared to an untreated control after at least 7 days of the administration, after at least 14 days of the administration, after at least 1 month of the administration, after at least 2 months of the administration, after at least 3 months of the administration, or after at least 4 months of the administration. According to another embodiment, the therapeutic amount of the N-acetyl-L-cysteine (NAC) is effective to increase number of FoxP3+CD8+CD25+ T cells compared to an untreated control after at least 7 days of the administration, after at least 14 days of the administration, after at least 1 month of the administration, after at least 2 months of the administration, after at least 3 months of the administration, or after at least 4 months of the administration. According to another embodiment, the therapeutic amount of the N-acetyl-L-cysteine (NAC) is effective to reduce cognitive/inattentive component of attention deficit and hyperactivity (ADHD) self-report scale (ASRS) compared to an untreated control after at least 7 days of the administration, after at least 14 days of the administration, after at least 1 month of the administration, after at least 2 months of the administration, after at least 3 months of the administration, or after at least 4 months of the administration.

According to another embodiment, the pharmaceutical composition further a second packaging material containing at least one additional therapeutic agent selected from the group consisting of a non-steroidal anti-inflammatory agent, an antimalarial agent, a corticosteroid, a cytotoxic agent, an immunosuppressive agent, a biologic, or a combination thereof. According to another embodiment, the non-steroidal anti-inflammatory agent is selected from the group consisting of aspirin, arthopan, celecoxib, diclofenac, etodolac, fenprofen, flurbiprofen, ibuprofen, ketoprofen, meclofamate, meloxicam, nabumetone, naproxen, oxaprozin, piroxicam, rofecoxib, sulindac, tolmetin, acetaminophen, or a combination thereof. According to another embodiment, the antimalarial agent is selected from the group consisting of hydroxycloroquine, chloroquine, quinicrine, or a combination thereof. According to another embodiment, the corticosteroid is in the form of a topical cream or ointment, a tablet, or an intravenous formulation. According to another embodiment, the topical cream is selected from the group consisting of clobetasol, halobetasol, hydrocortisone, triamcinolone, betamethasone, fluocinolone, fluocinonide, or a combination thereof. According to another embodiment, the tablet is selected from the group consisting of prednisone, prednisolone, ethylprednisone, or a combination thereof. According to another embodiment, the intravenous formulation is selected from the group consisting of methylprednisone, hydrocortisone, or a combination thereof. According to another embodiment, the cytotoxic agent is selected from the group consisting of azathioprine, cyclophosphamide, mycophenolate mofetil, cyclosporine A, methotrexate, chlorambucil, or a combination thereof. According to another embodiment, the immunosuppressive agent is selected from the group consisting of azathioprine, cyclophosphamide, mycophenolate mofetil, cyclosporine A, methotrexate, chlorambucil, or a combination thereof. According to another embodiment, the biologic is selected from the group consisting of a B-cell target biologic, a T cell target biologic, a spleen tyrosine kinase antagonist, a tumor necrosis factor (TNF) antagonist, an interferon antagonist, an interleukin-6-receptor antagonist, or a combination thereof

According to another embodiment, the means (b) for administering the composition is a syringe, a nebulizer, an inhaler, a dropper, a tablet, a pill, a gel, a troche, a lozenge, an aqueous suspension, an oily suspension, a capsule, a syrup, an emulsion, a cream, a patch, an injectable solution, a granule, a bead, an implant, a suppository, an insert, or a combination thereof. According to another embodiment, the kit further comprises instructions for use. According to another embodiment, at least one of the first or second packaging material is selected from the group consisting of a box, a pouch, a vial, a bottle, a tube, a blister pack, or a combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the effect of NAC and placebo on disease activity, as measured by SLEDAI (FIG. 1A), BILAG (FIG. 1B), and FAS scores (FIG. 1C), in 36 SLE patients exposed to placebo (n=9), 1.2 g/day NAC (NAC Dose 1, n=9), 2.4 g/day NAC (NAC Dose 2, n=9), 4.8 g/day NAC (NAC Dose 3, n=9), or all doses of NAC considered together (n=27). Data represent mean±SEM. p values reflect comparison of pretreatment values (visit 1) to values after treatment for 1 month (visit 2), 2 months (visit 3), 3 months (visit 4), or 4 months (visit 5, 3 months of treatment followed by 1 month washout) using two-tailed paired t-test.

FIG. 2 shows the effect of NAC on GSH of whole blood (WB) and peripheral blood lymphocytes (PBL) in patients with SLE. FIG. 2A: HPLC analysis of GSH in whole blood (WB) and peripheral blood lymphocytes (PBL) of untreated SLE patients (n=36) and healthy controls matched for age, gender, and ethnicity (n=42). p value reflects comparison with two-tailed unpaired t-test. FIG. 2B: Effect of NAC and placebo on GSH levels in whole blood of lupus patients. p values reflect comparison with two-tailed paired t-test. FIG. 2C: Effect of NAC and placebo on GSH levels in PBL of lupus patients. p values reflect comparison with two-tailed paired t-test.

FIG. 3 shows the effect of NAC on Δψₘ (FIG. 3A: DiOC⁶ fluorescence), mitochondrial mass (FIG. 3B: NAO fluorescence), and H₂O₂ levels were measured in T cells rested in culture for 16 h (FIG. 3C: DCF fluorescence). NO production (FIG. 3D: DAF-FM fluorescence), and mitochondrial mass were measured in T cell subsets following CD3/CD28 stimulation for 16 h (FIG. 3E: NAO fluorescence). FIG. 3F: Spontaneous apoptosis rate was enumerated by the percentage of Ann V+/PI− T cells after culture for 16 h. FIG. 3G: Activation-induced apoptosis was assessed following CD3/CD28 co-stimulation for 16 h. Visits: visit 1, before 1^{st} NAC dose; visit 2, after 1-month treatment; visit 3, after 2-month treatment; visit 4, after 3-month treatment; visit 5, after 1-month washout. p values reflect comparison to visit 1 using two-tailed paired t-test.

FIG. 4 shows the detection of increased mTOR activity via phosphorylation of S6 ribosomal protein (pS6-RP) in T-cell subsets from lupus and matched controls. A) Assessment of pS6-RP in CD3⁺, CD4⁺, CD8⁺, and DN T cells from control (blue histograms) and lupus donors (red histograms). Blue/red values show the percentage of cell populations with increased mTOR activity in control and lupus T-cell subsets, respectively. B) Cumulative analysis of mTOR activity in T-cell subsets of all lupus patients relative to all healthy controls. Values represent mean±SEM of cell populations with increased mTOR activity. p values reflect comparison of lupus and healthy donors with unpaired two-tailed t-test before treatment. C) Effect of NAC on mTOR activity measured by the prevalence of pS6-RP^{hi} T cells in lupus patients exposed to all doses considered together. p values reflect comparison to pre-treatment visit 1 using two-tailed paired t-test. D) Effect of NAC on CD3/CD28-induced mTOR activity in T cell subsets of lupus patients exposed to all doses considered together. p values reflect comparison to pre-treatment visit 1 using two-tailed paired t-test.

FIG. 5 shows the simulation of FoxP3 expression by NAC in lupus T cells. A) FoxP3 expression in CD4⁺/CD25⁺ and CD8⁺/CD25⁺ T cell subsets of lupus and control donors matched for age, gender, and ethnicity by flow cytometry. Red and blue values indicate percentage of FoxP3⁺ cells in lupus and control donors, respectively. B) Cumulative analysis of FoxP3 expression in CD25⁺ T-cell subsets in lupus subjects and matched controls. p values reflect comparison with two-tailed unpaired t-test. C) Effect of NAC on Foxp3 expression in CD25⁺ T cell subsets of lupus patients exposed to all doses considered together. p values reflect comparison with two-tailed paired t-test.

FIG. 6 shows a schematic functional hierarchy of metabolic biomarkers of T-cell dysfunction in patients with SLE. MHP is caused by exposure to nitric oxide (NO). De novo synthesis of NO and maintenance of GSH in reduced form are both dependent on the production of NADPH by the pentose phosphate pathway (PPP). MHP causes mTOR activation which in turn controls the expression of the transcription factor FoxP3.

FIG. 7 shows ASRS A (cognitive/inattentive), ASRS B (hyperactivity/impulsive), and total ASRS scores (ASRS Total) in patients with SLE and healthy controls matched for age within 10 years, gender, and ethnicity. Left panel, Analysis of cohort I comprising 24 SLE patients and 22 healthy subjects enrolled in a treatment trial of NAC (IND No: 101,320; clinicaltrials.gov identifier: NCT00775476). Middle panel: Analysis of cohort II comprising 25 SLE patients and 24 healthy subjects. Right panel, Analysis of cohorts I and II combined. Asterisks indicate p<0.05 comparing SLE and control subjects with two-tailed unpaired t-test.

FIG. 8 shows correlation of ASRS A and ASRS B scores with SLEDAI, BILAG, and FAS in 49 patients with SLE. Pearson's r values are shown for correlations with p<0.05.

FIG. 9 shows the effect of NAC and placebo on ASRS scores (ASRS total, left panel; ASRS A inattentive components, right panel) in 24 SLE patients exposed to placebo (n=6), 2.4 g/day NAC (NAC Dose 2; n=9), 4.8 g/day NAC (NAC Dose 3; n=9), or NAC Doses 2 and 3 considered together (NAC All doses; n=18). Data represent mean±SEM. p values reflect comparison of pretreatment values (visit 1) to values after treatment for 1 month (visit 2), 2 months (visit 3), 3 months (visit 4), or 4 months (visit 5, 3 months of treatment followed by 1 month washout) using two-tailed paired t-test.

### GLOSSARY

The term “absolute configuration” refers to the spatial arrangement of the atoms of a chiral molecular entity (or group) and its stereochemical description, for example, R or S.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “active” as used herein refers to the ingredient, component or constituent of the compositions of the described invention responsible for the intended therapeutic effect.

The terms “active agent” or “active ingredient” as used herein refer to the ingredient, component or constituent of the compositions of the described invention responsible for the intended therapeutic effect.

The term “additive effect”, as used herein, refers to a combined effect of two chemicals that is equal to the sum of the effect of each agent given alone.

The term “administer” as used herein means to give or to apply. The term “administering” as used herein includes in vivo administration, as well as administration directly to tissue ex vivo. Generally, compositions may be administered systemically either orally, buccally, parenterally, topically, by inhalation or insufflation (i.e., through the mouth or through the nose), administered rectally in dosage unit formulations containing conventional nontoxic pharmaceutically acceptable carriers, adjuvants, and vehicles as desired, or administered locally by means such as, but not limited to, injection, implantation, grafting, topical application, or parenterally.

The term “adverse event” (AE), as used herein, refers to any undesirable change from a patient's baseline condition associated with the use of a medical product in a patient. An undesirable change refers to any unfavorable or unintended sign including, but are not limited to, an abnormal laboratory finding, symptom or disease that occurs during the course of a study, whether or not considered related to the study drug, etc.

The term “agonist” as used herein refers to a chemical substance capable of activating a receptor to induce a full or partial pharmacological response. Receptors can be activated or inactivated by either endogenous or exogenous agonists and antagonists, resulting in stimulating or inhibiting a biological response. A physiological agonist is a substance that creates the same bodily responses, but does not bind to the same receptor. An endogenous agonist for a particular receptor is a compound naturally produced by the body which binds to and activates that receptor. A superagonist is a compound that is capable of producing a greater maximal response than the endogenous agonist for the target receptor, and thus an efficiency greater than 100%. This does not necessarily mean that it is more potent than the endogenous agonist, but is rather a comparison of the maximum possible response that can be produced inside a cell following receptor binding. Full agonists bind and activate a receptor, displaying full efficacy at that receptor. Partial agonists also bind and activate a given receptor, but have only partial efficacy at the receptor relative to a full agonist. An inverse agonist is an agent which binds to the same receptor binding-site as an agonist for that receptor and reverses constitutive activity of receptors. Inverse agonists exert the opposite pharmacological effect of a receptor agonist. An irreversible agonist is a type of agonist that binds permanently to a receptor in such a manner that the receptor is permanently activated. It is distinct from a mere agonist in that the association of an agonist to a receptor is reversible, whereas the binding of an irreversible agonist to a receptor is believed to be irreversible. This causes the compound to produce a brief burst of agonist activity, followed by desensitization and internalization of the receptor, which with long-term treatment produces an effect more like an antagonist. A selective agonist is specific for one certain type of receptor.

The term “antagonist” as used herein refers to a substance that interferes with the effects of another substance. Functional or physiological antagonism occurs when two substances produce opposite effects on the same physiological function. Chemical antagonism or inactivation is a reaction between two substances to neutralize their effects. Dispositional antagonism is the alteration of the disposition of a substance (its absorption, biotransformation, distribution, or excretion) so that less of the agent reaches the target or its persistence there is reduced. Antagonism at the receptor for a substance entails the blockade of the effect of an antagonist with an appropriate antagonist that competes for the same site.

The term “anti-inflammatory agent” as used herein refers to an agent that reduces inflammation. The term “steroidal anti-inflammatory agent”, as used herein, refer to any one of numerous compounds containing a 17-carbon 4-ring system and includes the sterols, various hormones (as anabolic steroids), and glycosides. The term “non-steroidal anti-inflammatory agents” refers to a large group of agents that are aspirin-like in their action, including ibuprofen (Advil)®, naproxen sodium (Aleve)®, and acetaminophen (Tylenol)®.

The term “antimalarial agent” as used herein refers to an agent that prevents or cures malaria or that inhibits or destroys malarial parasites.

The term “anti-oxidant agent” as used herein refers to a substance that inhibits oxidation or reactions promoted by oxygen or peroxides. Non-limiting examples of anti-oxidants that are usable in the context of the described invention include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox®), gallic acid and its alkyl esters (for example, propyl gallate), uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, glycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

The term “apoptotic cell” as used herein refers to a cell that undergoes programmed cell death and is characterized by fragmented high molecular-weight deoxyribose nucleic acid (DNA). Apoptotic cells are detected by staining of double-stranded DNA such as fluorophore-conjugated propidium iodide. The term “apoptotic rate” as used herein refers to the percentage of apoptotic cells in a given cell sample. The term “spontaneous apoptotic rate” as used herein refers to the percentage of apoptotic cells in a non-treated cell sample. The term “activation-induced apoptotic rate” as used herein refers to the percentage of apoptotic cells in a cell sample in which apoptosis is induced, for example with co-stimulation with anti-CD3/anti-CD28.

The term “binder” refers to substances that bind or “glue” powders together and make them cohesive by forming granules, thus serving as the “adhesive” in the formulation. Binders add cohesive strength already available in the diluent or bulking agent. Suitable binders include sugars such as sucrose; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2% to about 20% by weight of the composition, more preferably from about 3% to about 10% by weight, even more preferably from about 3% to about 6% by weight.

The term “bioavailability” refers to the rate and extent to which the active drug ingredient or therapeutic moiety is absorbed into the systemic circulation from an administered dosage form as compared to a standard or control.

The term “biologic” refers to a medicinal preparation that is created by biological processes rather than chemical synthesis. Exemplary biologics include vaccine, monoclonal antibodies, cell preparations, tissue preparations, recombinant proteins, etc.

The terms “buccal”, “buccally” or “buccal administration” are used interchangeably to refer to administration of a medicinal formulation between the cheek and gums.

The terms “buffer” or “buffering agent” are used interchangeably to mean an excipient that stabilizes pH of a composition, such as a pharmaceutical composition. Exemplary buffers include but are not limited to borate buffers, histidine buffers, citrate buffers, succinate buffers, acetate buffers, tartrate buffers, phosphate buffers, Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

The term “capsule” refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

The term “carrier” as used herein describes a material that does not cause significant irritation to an organism and does not abrogate the biological activity and properties of the active compound of the composition of the described invention. Carriers must be of sufficiently high purity and of sufficiently low toxicity to render them suitable for administration to the mammal being treated. The carrier can be inert, or it can possess pharmaceutical benefits, cosmetic benefits or both. The terms “excipient”, “carrier”, or “vehicle” are used interchangeably to refer to carrier materials suitable for formulation and administration of pharmaceutically acceptable compositions described herein. Carriers and vehicles useful herein include any such materials know in the art which are nontoxic and do not interact with other components.

The term “cellular cast” as used herein refers to an elongated or cylindrical mold formed in a tubular structure having a hyaline matrix with the inclusion of cells, observed in histological preparations of urine or sputum.

The term “chiral” is used to describe asymmetric molecules (with four different substituent groups) that are nonsuperposable since they are mirror images of each other and therefore has the property of chirality. Such molecules are also called enantiomers and are characterized by optical activity.

The term “chirality” refers to the geometric property of a rigid object (or spatial arrangement of points or atoms) of being non-superposable on its mirror image; such an object has no symmetry elements of the second kind (a mirror plane, σ=S1, a center of inversion, i=S2, a rotation-reflection axis, S2n). If the object is superposable on its mirror image the object is described as being achiral.

The term “chirality axis” refers to an axis about which a set of ligands is held so that it results in a spatial arrangement which is not superposable on its mirror image. For example, with an allene abC=C=Ccd the chiral axis is defined by the C=C=C bonds; and with an ortho-substituted biphenyl C-1, C-1′, C-4 and C-4′ lie on the chiral axis.

The term “chirality center” or a “chiral center” refers to an atom holding a set of ligands in a spatial arrangement, which is not superposable on its mirror image. A chirality center may be considered a generalized extension of the concept of the asymmetric carbon atom to central atoms of any element.

The terms “chiroptic” or “chiroptical” refer to the optical techniques (using refraction, absorption or emission of anisotropic radiation) for investigating chiral substances (for example, measurements of optical rotation at a fixed wavelength, optical rotary dispersion (ORD), circular dichroism (CD) and circular polarization of luminescence (CPL).

The term “chirotopic” refers to the an atom (or point, group, face, etc. in a molecular model) that resides within a chiral environment. One that resides within an achiral environment has been called achirotopic.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

The term “Cohen's D” as used herein refers to an effect size for the comparison between two means. It is defined as the difference between two means divided by a standard deviation for a given data. It is used for estimating sample sizes. A lower Cohen's D indicates larger sample size and vice versa. The term “effect size” as used herein refers to a measure of the strength of a phenomenon, for example, the relationship between two variables in a statistical population. (Kelley, K. and Preacher, K. J., “On effect size,” Psychological Methods, 17(2): 137-152 (2012); Cohen, J., “Statistical power analysis for the behavioral sciences,” Second Ed., Lawrence Erlbaum Associates, (1988)).

The term “coloring agents” refers to excipients that provide coloration to the composition or the dosage form. Such excipients can include food grade dyes and food grade dyes adsorbed onto a suitable adsorbent such as clay or aluminum oxide. The amount of the coloring agent can vary from about 0.1% to about 5% by weight of the composition, preferably from about 0.1% to about 1%.

The term “composition” as used herein refers to a material formed of two or more substances.

The term “condition”, as used herein, refers to a variety of health states and is meant to include disorders or diseases caused by any underlying mechanism or disorder, injury, and the promotion of healthy tissues and organs.

The term “cytotoxic agent” as used herein refers to an agent that is destructive or detrimental to cell viability.

The term “delayed release” is used herein in its conventional sense to refer to a drug formulation in which there is a time delay between administration of the formulation and the release of the drug there from. “Delayed release” may or may not involve gradual release of drug over an extended period of time, and thus may or may not be “sustained release.”

The term “derivative” as used herein refers to a compound that may be produced from another compound of similar structure in one or more steps. A “derivative” or “derivatives” of a compound retains at least a degree of the desired function of the compound. Accordingly, an alternate term for “derivative” may be “functional derivative.” A derivative of N-acetylcysteine has the same biological activity as does N-acetylcysteine.

The derivatives of N-acetylcysteine, for example, contain one or more functional groups (e.g., aliphatic, aromatic, heterocyclic radicals, epoxides, and/or arene oxides) incorporated into N-acetylcysteine. According to another embodiment, the derivatives of N-acetylcysteine disclosed herein also comprise “prodrugs” of N-acetylcysteine, which are either active in the prodrug form or are cleaved in vivo to the parent active compound. According to another embodiment, the derivatives of N-acetylcysteine also includes any pharmaceutically acceptable salt, ester, solvate, hydrate or any other compound, which, upon administration to the recipient, is capable of providing (directly or indirectly) N-acetylcysteine.

As used herein the term “diagnose” refers to the act or process of identifying or determining a disease or condition in a mammal or the cause of a disease or condition by the evaluation of the signs and symptoms of the disease or disorder.

The term “diluent” refers to substances that usually make up the major portion of the composition or dosage form. Exemplary diluents include, but are not limited to, sugars such as lactose, sucrose, mannitol and sorbitol; starches derived from wheat, corn, rice and potato; and celluloses such as microcrystalline cellulose. The amount of diluent in the composition can range from about 10% to about 90% by weight of the total composition, preferably from about 25% to about 75%, more preferably from about 30% to about 60% by weight, even more preferably from about 12% to about 60%.

The term “disintegrant” refers to materials added to the composition to help it break apart (disintegrate) and release the medicaments. Suitable disintegrants include starches; “cold water soluble” modified starches such as sodium carboxymethyl starch; natural and synthetic gums such as locust bean, karaya, guar, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2 to about 15% by weight of the composition, more preferably from about 4 to about 10% by weight.

The term “disease” or “disorder”, as used herein, refers to an impairment of health or a condition of abnormal functioning.

The term “disease activity” as used herein is defined as the reversible manifestations of the underlying inflammatory process in a lupus condition, such as systemic lupus eythematosus (SLE). It is a reflection of the type and severity of organ involvement at each point in time.

The term “disease activity index” as used herein refers to a research tool used to quantify the extent of symptoms associated with a lupus condition in a given patient.

The terms “dose” and “dosage” are used interchangeably and mean the quantity of a drug or other remedy to be taken or applied all at one time or in fractional amounts within a given period.

The term “double negative T cell” as used herein refers to CD4− CD8− T cells.

The term “drug” as used herein refers to a therapeutic agent or any substance, other than food, used in the prevention, diagnosis, alleviation, treatment, or cure of disease. The terms “drug” and “pharmaceutical” also are used interchangeably to refer to a pharmacologically active substance or composition. These terms of art are well-known in the pharmaceutical and medicinal arts.

The term “dye” (also referred to as “fluorochrome” or “fluorophore”) as used herein refers to a component of a molecule which causes the molecule to be fluorescent. The component is a functional group in the molecule that absorbs energy of a specific wavelength and re-emits energy at a different (but equally specific) wavelength. The amount and wavelength of the emitted energy depend on both the dye and the chemical environment of the dye. Many dyes are known, including, but not limited to, FITC, R-phycoerythrin (PE), PE-Texas Red Tandem, PE-Cy5 Tandem, propidium iodem, EGFP, EYGP, ECF, DsRed, allophycocyanin (APC), PerCp, SYTOX Green, courmarin, Alexa Fluors (350, 430, 488, 532, 546, 555, 568, 594, 633, 647, 660, 680, 700, 750), Cy2, Cy3, Cy3.5, Cy5, Cy5.5, Cy7, Hoechst 33342, DAPI, Hoechst 33258, SYTOX Blue, chromomycin A3, mithramycin, YOYO-1, SYTOX Orange, ethidium bromide, 7-AAD, acridine orange, TOTO-1, TO-PRO-1, thiazole orange, TOTO-3, TO-PRO-3, thiazole orange, propidium iodide (PI), LDS 751, Indo-1, Fluo-3, DCFH, DHR, SNARF, Y66F, Y66H, EBFP, GFPuv, ECFP, GFP, AmCyan1, Y77W, S65A, S65C, S65L, S65T, ZsGreen1, ZsYellow1, DsRed2, DsRed monomer, AsRed2, mRFP1, HcRed1, monochlorobimane, calcein, the DyLight Fluors, cyanine, hydroxycoumarin, aminocoumarin, methoxycoumarin, Cascade Blue, Lucifer Yellow, NBD, PE-Cy5 conjugates, PE-Cy7 conjugates, APC-Cy7 conjugates, Red 613, fluorescein, FluorX, BODIDY-FL, TRITC, Xrhodamine, Lissamine Rhodamine B, Texas Red, TruRed, and derivatives thereof

The term “effective amount” refers to the amount necessary or sufficient to realize a desired biologic effect.

The term “enantiomer” as used herein refers to one of a pair of optical isomers containing one or more asymmetric carbons (C*) whose molecular configurations have left- and right-hand (chiral) configurations. Enantiomers have identical physical properties, except as to the direction of rotation of the plane of polarized light. For example, glyceraldehyde and its mirror image have identical melting points, boiling points, densities, refractive indexes, and any other physical constant one might measure, expect that they are non-superimposable mirror images and one rotates the plane-polarized light to the right, while the other to the left by the same amount of rotation.

[chemical structure]

The term “erythema′ as used herein refers to redness due to capillary dilation.

The term “flow cytometry” as used herein refers to a tool for interrogating the phenotype and characteristics of cells. Flow cytometry is a system for sensing cells or particles as they move in a liquid stream through a laser (light amplification by stimulated emission of radiation)/light beam past a sensing area. The relative light-scattering and color-discriminated fluorescence of the microscopic particles is measured. Analysis and differentiation of the cells is based on size, granularity, and whether the cells is carrying fluorescent molecules in the form of either antibodies or dyes. As the cell passes through the laser beam, light is scattered in all directions, and the light scattered in the forward direction at low angles (0.5-10°) from the axis is proportional to the square of the radius of a sphere and so to the size of the cell or particle. Light may enter the cell; thus, the 90° light (right-angled, side) scatter may be labled with fluorochrome-linked antibodies or stained with fluorescent membrane, cytoplasmic, or nuclear dyes. Thus, the differentiation of cell types, the presence of membrane receptors and antigens, membrane potential, pH, enzyme activity, and DNA content may be facilitated. Flow cytometers are multiparameter, recording several measurements on each cell; therefore, it is possible to identify a homogeneous subpopulation within a heterogeneous population (Marion G. Macey, Flow cytometry: principles and applications, Humana Press, 2007).

The term “fluorescence” as used herein refers to the result of a three-state process that occurs in certain molecules, generally referred to as “fluorophores” or “fluorescent dyes,” when a molecule or nanostructure relaxes to its ground state after being electrically excited. Stage 1 involves the excitation of a fluorophore through the absorption of light energy; Stage 2 involves a transient excited lifetime with some loss of energy; and Stage 3 involves the return of the fluorophore to its ground state accompanied by the emission of light.

The term “fluorescent-activated cell sorting” (also referred to as “FACS”) as used herein refers to a method for sorting a heterogeneous mixture of biological cells into one or more containers, one cell at a time, based upon the specific light scattering and fluorescent characteristics of each cell.

The term “formulation” as used herein refers to a mixture prepared according to a formula, recipe or procedure. As used herein, the terms “formulation” and “composition” are used interchangeably.

The term “fractionate” and its various grammatical forms as used herein refers to separating or dividing into component parts, fragments, or divisions.

The term “hemolytic anemia” as used herein refers to any condition in which the number of erythrocytes (red blood cells) per mm³ or the amount of hemoglobin in 100 ml of blood is less than normal resulting from the destruction of erythrocytes.

The term “hypertension” as used herein refers to high systemic blood pressure, a transitory or sustained elevation of systemic blood pressure to a level likely to induce cardiovascular damage or other adverse consequences.

The term “immunosuppressive agent” as used herein refers to an agent that prevents or interferes with the development of immunologic response.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses. The classic signs of inflammation are pain (dolor), heat (calor), redness (rubor), swelling (tumor), and loss of function (functio laesa). Histologically, inflammation involves a complex series of events, including dilatation of arterioles, capillaries, and venules, with increased permeability and blood flow; exudation of fluids, including plasma proteins; and leukocytic migration into the inflammatory focus. The term “acute inflammation” as used herein, refers to inflammation, usually of sudden onset, characterized by the classical signs, with predominance of the vascular and exudative processes. The term “chronic inflammation” as used herein refers to inflammation of slow progress and marked chiefly by the formation of new connective tissue; it may be a continuation of an acute form or a prolonged low-grade form, and usually causes permanent tissue damage.

Regardless of the initiating agent, the physiologic changes accompanying acute inflammation encompass four main features: (1) vasodilation, which results in a net increase in blood flow, is one of the earliest physical responses to acute tissue injury; (2) in response to inflammatory stimuli, endothelial cells lining the venules contract, widening the intracellular junctions to produce gaps, leading to increased vascular permeability which permits leakage of plasma proteins and blood cells out of blood vessels; (3) inflammation often is characterized by a strong infiltration of leukocytes at the site of inflammation, particularly neutrophils (polymorphonuclear cells). These cells promote tissue damage by releasing toxic substances at the vascular wall or in uninjured tissue; and (4) fever, produced by pyrogens released from leukocytes in response to specific stimuli.

During the inflammatory process, soluble inflammatory mediators of the inflammatory response work together with cellular components in a systemic fashion in the attempt to contain and eliminate the agents causing physical distress.

The term “inflammatory mediators” as used herein refers to the molecular mediators of the inflammatory process. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, plateletactivating factor (PAF), peptides and amines, including, but not limited to, histamine, serotonin, and neuropeptides, proinflammatory cytokines, including, but not limited to, interleukin-1, interleukin-4, interleukin-6, interleukin-8, tumor necrosis factor (TNF), interferon-gamma, and interleukin 12.

The term “inhibiting” as used herein refers to reducing or modulating the chemical or biological activity of a substance or compound.

The term “injection”, as used herein, refers to introduction into subcutaneous tissue, or muscular tissue, a vein, an artery, or other canals or cavities in the body by force.

The term “injury,” as used herein, refers to damage or harm to a structure or function of the body caused by an outside agent or force, which may be physical or chemical.

The terms “in the body”, “void volume”, “resection pocket”, “excavation”, “injection site”, “deposition site” or “implant site” or “site of delivery” as used herein are meant to include all tissues of the body without limit, and may refer to spaces formed therein from injections, surgical incisions, tumor or tissue removal, tissue injuries, abscess formation, or any other similar cavity, space, or pocket formed thus by action of clinical assessment, treatment or physiologic response to disease or pathology as non-limiting examples thereof

The term “insufflation” as used herein refers to delivery by inhalation through the nose or mouth.

The term “lubricant” refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d′l-leucine. Lubricants are usually added at the very last step before compression, since they must be present on the surfaces of the granules and in between them and the parts of the tablet press. The amount of lubricant in the composition can range from about 0.2% to about 5% by weight of the composition, preferably from about 0.5% to about 2%, more preferably from about 0.3% to about 1.5% by weight.

The term “leukopenia” as used herein refers to a condition in which the total number of leukocytes in circulating blood is less than normal.

The term “lupus condition” as used herein refers to lupus erythematosus, an autoimmune multisystem disorder of unknown etiology characterized by the presence of antinuclear antibodies (ANAs) and associated with inflammation that may be chronic or subacute.

The term “lymphopenia” as used herein refers to a condition in which there is a reduction in the number of lymphocytes in circulating blood as compared to normal conditions.

The term “maximum daily adult dose” as used herein in the context of a toxicity study refers to the highest dose of a drug per day that does not produce unacceptable toxicity in an adult of average body weight of 70 kg. The term “maximum daily pediatric dose” as used herein in the context of a toxicity study refers to the highest dose of a drug per day that does not produce unacceptable toxicity in a child of average body weight of 10 kg.

The term “mitochondrial hyperpolarization” as used herein refers to generation of the mitochondrial membrane potential (Δψₘ)). It is the result of an electrochemical gradient maintained by two transport systems—the electron transport chain and the F₀F₁-ATPase complex.

The term “mitochondrial mass” as used herein refers to the total content of mitochondria. Mitochondrial mass can be measured by the use of fluorescent dyes such as nonyl acridine orange (NAO) which become fluorescent once accumulated to a certain concentration in the mitochondrial lipid environment.

The term “mitochondrial membrane potential (Δψₘ)” as used herein refers to the difference in electric potential across the inner mitochondrial membrane with the inside negative and outside positive as a result of the net outflow of positive ions, resulting from the pumping of H+ across the inner mitochondrial membrane from the matrix to the intermembrane space that driven by the energetically favorable flow of electrons mediated by an electrochemical gradient across the inner mitochondrial membrane.

The term “modify” as used herein means to change, vary, adjust, temper, alter, affect or regulate to a certain measure or proportion in one or more particulars.

The term “modifying agent” as used herein refers to a substance, composition, extract, botanical ingredient, botanical extract, botanical constituent, therapeutic component, active constituent, therapeutic agent, drug, metabolite, active agent, protein, non-therapeutic component, non-active constituent, non-therapeutic agent, or non-active agent that reduces, lessens in degree or extent, or moderates the form, symptoms, signs, qualities, character or properties of a condition, state, disorder, disease, symptom or syndrome.

The term “modulate” as used herein means to regulate, alter, adapt, or adjust to a certain measure or proportion.

The term “non-oral administration” represents any method of administration in which a composition is not provided in a solid or liquid oral dosage form, wherein such solid or liquid oral dosage form is traditionally intended to substantially release and or deliver the drug in the gastrointestinal tract beyond the mouth and/or buccal cavity.

The term “optical rotation” refers to the change of direction of the plane of polarized light to either the right or the left as it passes through a molecule containing one or more asymmetric carbon atoms or chirality centers. The direction of rotation, if to the right, is indicated by either a plus sign (+) or a d−; if to the left, by a minus (−) or an l−. Molecules having a right-handed configuration (D) usually are dextrorotatory, D(+), but may be levorotatory, L(−). Molecules having left-handed configuration (L) are usually levorotatory, L(−), but may be dextrorotatory, D(+). Compounds with this property are said to be optically active and are termed optical isomers. The amount of rotation of the plane of polarized light varies with tye molecule but is the same for any two isomers, though in opposite directions.

As used herein, the term “optionally” means that the subsequently described event(s) may or may not occur, and includes both event(s) which occur and events that do not occur.

As used herein, the terms “oral” or “orally” refer to the introduction into the body by mouth whereby absorption occurs in one or more of the following areas of the body: the mouth, stomach, small intestine, lungs (also specifically referred to as inhalation), and the small blood vessels under the tongue (also specifically referred to as sublingually).

The term “parenteral” as used herein refers to introduction into the body by way of an injection (i.e., administration by injection) outside the gastrointestinal tract, including, for example, subcutaneously (i.e., an injection beneath the skin), intramuscularly (i.e., an injection into a muscle); intravenously (i.e., an injection into a vein), intrathecally (i.e., an injection into the space around the spinal cord), intrasternal injection, or by infusion techniques. A parenterally administered composition is delivered using a needle, e.g., a surgical needle. The term “surgical needle” as used herein, refers to any needle adapted for delivery of fluid (i.e., those capable of flow) compositions into a selected anatomical structure. Injectable preparations, such as sterile injectable aqueous or oleaginous suspensions, may be formulated according to the known art using suitable dispersing or wetting agents and suspending agents.

The term “pericarditis” as used herein refers to inflammation of the pericardial membrane of the heart.

The term “pharmaceutically acceptable carrier” as used herein refers to one or more compatible solid or liquid filler, diluent or encapsulating substance which is/are suitable for administration to a human or other vertebrate animal. The components of the pharmaceutical compositions also are capable of being commingled in a manner such that there is no interaction which would substantially impair the desired pharmaceutical efficiency.

The term “pharmaceutically acceptable salt” as used herein refers to those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002).

The term “pharmaceutical composition” is used herein to refer to a composition that is employed to prevent, reduce in intensity, cure or otherwise treat a target condition or disease.

The term “pharmacologic effect”, as used herein, refers to a result or consequence of exposure to an active agent.

The term “pleuritis” as used herein refers to an inflammation of the pleura membrane of the lung.

The phrase “powder for constitution” refers to powder blends containing the active ingredients and suitable diluents which can be suspended in water or juices.

The term “prognosis” as used herein refers to an expected future cause and outcome of a disease or disorder, based on medical knowledge.

The term “proteinuria” as used herein refers to the presence of urinary protein in an amount greater than 0.3 g in a 24 hour urine collection or in concentrations greater than 1 g/L (1+ to 2+ by standard turbidometric methods) in a random urine collection on two or more occasions at least 6 hours apart.

The term “psychosis” as used herein refers to a mental or behavioral disorder causing gross distortion or disorientation of a person's mental capacity, affective response, and capacity to recognize reality, communicate and relate to others to the extent of interfering with the person's capacity to cope with the ordinary demands of everyday life.

The terms “reactive oxygen species (ROS)” or “reactive oxygen intermediates (ROIs)” are used interchangeably to mean oxygen radicals and peroxides that are derived from the metabolism of oxygen and exist inherently in all aerobic organisms. ROS comprise oxygen derived small molecules such as oxygen radicals: superoxide, hydroxyl, peroxyl, and alkoxyl; or the nonradicals: hypochlorous acid, ozone, singlet oxygen, and hydrogen peroxide. The term “oxygen radicals” as used herein refers to any oxygen species that carries an unpaired electron (except free oxygen). The transfer of electrons to oxygen also may lead to the production of toxic free radical species. The best documented of these is the superoxide radical. Oxygen radicals, such as the hydroxyl radical (OH⁻) and the superoxide ion (O₂.⁻) are very powerful oxidizing agents that cause structural damage to proteins, lipids and nucleic acids. The free radical superoxide anion, a product of normal cellular metabolism, is produced mainly in mitochondria because of incomplete reduction of oxygen. The superoxide radical, although unreactive compared with many other radicals, may be converted by biological systems into other more reactive species, such as peroxyl (ROO⁻), alkoxyl (RO⁻) and hydroxyl (OH⁻) radicals. ROS generation can occur either as a by-product of cellular metabolism (e.g., in mitochondria through autoxidation of respiratory chain components) or it can be created by enzymes with the primary function of ROS generation. (M. Rojkind et al, Cellular & Molec. Life Sci. 59(11): 1872-1891 (2002)).

The term “racemate” as used herein refers to an equimolar mixture of two optically active components that neutralize the optical effect of each other and is therefore optically inactive.

The terms “rectal” or “rectally” are used interchangeably to refer to introduction into the body through the rectum where absorption occurs through the walls of the rectum.

The term “reduce” or “reducing” as used herein refers to a diminution, a decrease, an attenuation, limitation or abatement of the degree, intensity, extent, size, amount, density, number or occurrence of disorder in individuals at risk of developing the disorder.

The term “seizure” as used herein refers to an epileptic attack characterized by loss of consciousness that may vary from complex abnormalities of behavior including generalized or focal convulsions to momentary spells of impaired consciousness.

The terms “stabilizing agent” and “stabilizer” are used interchangeably to mean a chemical or a compound that is added to a solution, mixture, suspension, or composition to maintain it in a stable or unchanging state.

The term “sublingual” as used herein refers to administration of a medicinal formulation under the tongue such that the active ingredient(s) can diffuse into the blood through the tissues under the tongue.

The terms “subject” or “individual” or “patient” are used interchangeably to refer to a member of an animal species of mammalian origin that may benefit from the administration of a drug composition or method of the described invention. Examples of subjects include humans, and other animals such as horses, pigs, cattle, dogs, cats, rabbits, and aquatic mammals.

The term “subject in need thereof” as used herein refers to a subject showing signs and symptoms of or susceptible to a lupus disorder.

The term “substantially pure”, as used herein, refers to a condition of a therapeutic agent such that it has been substantially separated from the substances with which it may be associated in living systems or during synthesis. According to some embodiments, a substantially pure therapeutic agent is at least 70% pure, at least 75% pure, at least 80% pure, at least 85% pure, at least 90% pure, at least 95% pure, at least 96% pure, at least 97% pure, at least 98% pure, or at least 99% pure.

The term “sustained release” (also referred to as “extended release”) is used herein in its conventional sense to refer to a drug formulation that provides for gradual release of a drug over an extended period of time, and that preferably, although not necessarily, results in substantially constant blood levels of a drug over an extended time period.

The terms “sweetening agent” or “sweetener” are used interchangeably to include for example saccharin sodium, dipotassium glycyrrhizate, aspartame and the like.

The term “syndrome,” as used herein, refers to a pattern of symptoms indicative of some disease or condition.

The term “symptom” as used herein refers to a phenomenon that arises from and accompanies a particular disease or disorder and serves as an indication of it.

The term “synergistic effect”, as used herein, refers to a combined effect of two chemicals, which is greater than the sum of the effects of each agent given alone.

The phrase “systemic administration”, as used herein, refers to administration of a therapeutic agent with a pharmacologic effect on the entire body. Systemic administration includes enteral administration (e.g. oral) through the gastrointestinal tract and parenteral administration (e.g. intravenous, intramuscular, etc.) outside the gastrointestinal tract.

The term “tablet” refers to a compressed or molded solid dosage form containing the active ingredients with suitable diluents. The tablet can be prepared by compression of mixtures or granulations obtained by wet granulation, dry granulation or by compaction.

The term “therapeutic agent” as used herein refers to a drug, molecule, composition or other substance that provides a therapeutic effect. The terms “therapeutic agent” and “active agent” are used interchangeably herein.

The terms “therapeutic amount”, “therapeutic effective amount” or an “amount effective” of one or more of the therapeutic agents is an amount that is sufficient to provide the intended benefit of treatment. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen may be planned which does not cause substantial toxicity and yet is effective to treat the particular subject. A therapeutic effective amount of the therapeutic agents that can be employed ranges from generally 0.1 mg/kg body weight and about 50 mg/kg body weight. A therapeutic effective amount for any particular application may vary depending on such factors as the disease or condition being treated, the particular therapeutic agent being administered, the size of the subject, or the severity of the disease or condition. One of ordinary skill in the art may determine empirically the effective amount of a particular inhibitor and/or other therapeutic agent without necessitating undue experimentation. It is preferred generally that a maximum dose be used, that is, the highest safe dose according to some medical judgment. However, dosage levels are based on a variety of factors, including the type of injury, the age, weight, sex, medical condition of the patient, the severity of the condition, the route of administration, and the particular therapeutic agent employed. Thus the dosage regimen may vary widely, but can be determined routinely by a surgeon using standard methods. “Dose” and “dosage” are used interchangeably herein.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50, which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect also may include, directly or indirectly, the arrest reduction or elimination of the progression of a disease manifestation.

The term “therapeutically effective amount” or an “amount effective” of one or more active agent(s) is an amount that is sufficient to provide the intended benefit of treatment. Dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods.

The term “thrombocytopenia” as used herein refers to a condition in which the number of platelets circulating in the blood is below the normal range of platelets.

The term “topical” refers to administration of a composition at, or immediately beneath, the point of application. The phrase “topically applying” describes application onto one or more surfaces(s) including epithelial surfaces. Topical administration, in contrast to transdermal administration, generally provides a local rather than a systemic effect.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

### DETAILED DESCRIPTION

The described invention relates to treatment of a lupus condition, such as systemic lupus erythematosus (SLE) comprising administering a pharmaceutical composition comprising a therapeutic amount of N-acetyl-L-cysteine (NAC). According to one embodiment, the therapeutic amount of NAC is effective to inhibit the mammalian target of rapamycin (mTOR), and thereby to treat the lupus condition without any significant side effect.

According to another embodiment, the pharmaceutical composition of the described invention is able to reduce a disease activity index of the lupus condition. According to one embodiment, the disease activity index is systemic lupus erythematosus disease activity index (SLEDAI). According to another embodiment, the disease activity index is British Isles Lupus Assessment Group (BILAG) score. According to another embodiment, the pharmaceutical composition of the described invention is able to treat fatigue associated with the lupus condition.

According to another embodiment, the pharmaceutical composition of the described invention is able to detect early and treat a neuropsychiatric complication associated with the lupus condition. According to one embodiment, the neuropsychiatric condition is attention deficit and hyperactivity disorder (ADHD). According to one embodiment, the therapeutic amount of N-acetyl-L-cysteine (NAC) is effective to reduce cognitive/inattentive component of attention deficit and hyperactivity (ADHD) self-report scale (ASRS).

According to another embodiment, the pharmaceutical composition of the described invention is able to reduce formation of immune complexes (ICs).

### Methods for Treating Systemic Lupus Erythematous (SLE)

According to one aspect, the present disclosure provides a method of treating a lupus condition in a subject in need thereof, comprising:

(a) providing a composition comprising a therapeutic amount of a compound N-acetyl-L-cysteine (NAC) of Formula I:

[chemical structure]

or a pharmaceutically acceptable salt, solvate, prodrug or a derivative thereof; and a pharmaceutically acceptable carrier; and

(b) administering the composition to the subject, wherein the therapeutic amount is effective to decrease activity of mammalian target of rapamycin (mTOR) and to treat one or more symptoms of the lupus condition.

According to one embodiment, the lupus condition is systemic lupus erythematosus (SLE). According to some such embodiments, the systemic lupus erythematosus (SLE) is characterized by at least four of American College of Rheumatology (ACR) criteria selected from the group consisting of a malar rash, a discoid rash, a photosensitivity rash, an oral ulcer, a nonerosive arthritic condition, pleuritis, pericarditis, a renal disorder, a neurologic disorder, a hematologic disorder, an immunologic disorder, or a positive antinuclear antibody test. According to some embodiments, the renal disorder is persistent proteinuria or a cellular cast. According to some embodiments, the neurologic disorder is a seizure or a psychosis. According to some embodiments, the hematologic disorder is hemolytic anemia, leucopenia, lymphopenia, or thrombocytopenia. According to another embodiment, the lupus condition is discoid lupus erythematosus. According to another embodiment, the lupus condition is neonatal lupus erythematosus. According to another embodiment, the lupus condition is drug-induced lupus erythematosus.

According to some embodiments, the therapeutic amount of N-acetyl-L-cysteine (NAC) is from about 1 mg/day to about 8000 mg/day. According to one embodiment, the therapeutic amount of N-acetyl-L-cysteine (NAC) is about 8000 mg/day. According to another embodiment, the therapeutic amount of N-acetyl-L-cysteine (NAC) is about 7800 mg/day. According to another embodiment, the therapeutic amount of N-acetyl-L-cysteine (NAC) is about 7600 mg/day. According to another embodiment, the therapeutic amount of N-acetyl-L-cysteine (NAC) is about 7400 mg/day. According to another embodiment, the therapeutic amount of N-acetyl-L-cysteine (NAC) is about 7200 mg/day. According to another embodiment, the therapeutic amount of N-acetyl-L-cysteine (NAC) is about 7000 mg/day. According to another embodiment, the therapeutic amount of N-acetyl-L-cysteine (NAC) is about 6800 mg/day. According to another embodiment, the therapeutic amount of N-acetyl-L-cysteine (NAC) is about 6600 mg/day. According to another embodiment, the therapeutic amount of N-acetyl-L-cysteine (NAC) is about 6400 mg/day. According to another embodiment, the therapeutic amount of N-acetyl-L-cysteine (NAC) is about 6200 mg/day. According to another embodiment, the therapeutic amount of N-acetyl-L-cysteine (NAC) is about 6000 mg/day. According to another embodiment, the therapeutic amount of N-acetyl-L-cysteine (NAC) is about 5800 mg/day. According to another embodiment, the therapeutic amount of N-acetyl-L-cysteine (NAC) is about 5600 mg/day. According to another embodiment, the therapeutic amount of N-acetyl-L-cysteine (NAC) is about 5400 mg/day. According to another embodiment, the therapeutic amount of N-acetyl-L-cysteine (NAC) is about 5200 mg/day. According to another embodiment, the therapeutic amount of N-acetyl-L-cysteine (NAC) is about 5000 mg/day. According to another embodiment, the therapeutic amount of N-acetyl-L-cysteine (NAC) is about 4800 mg/day. According to another embodiment, the therapeutic amount of N-acetyl-L-cysteine (NAC) is about 4600 mg/day. According to another embodiment, the therapeutic amount of N-acetyl-L-cysteine (NAC) is about 4400 mg/day. According to another embodiment, the therapeutic amount of N-acetyl-L-cysteine (NAC) is about 4200 mg/day. According to another embodiment, the therapeutic amount of N-acetyl-L-cysteine (NAC) is about 4000 mg/day. According to another embodiment, the therapeutic amount of N-acetyl-L-cysteine (NAC) is about 3800 mg/day. According to another embodiment, the therapeutic amount of N-acetyl-L-cysteine (NAC) is about 3600 mg/day. According to another embodiment, the therapeutic amount of N-acetyl-L-cysteine (NAC) is about 3400 mg/day. According to another embodiment, the therapeutic amount of N-acetyl-L-cysteine (NAC) is about 3200 mg/day. According to another embodiment, the therapeutic amount of N-acetyl-L-cysteine (NAC) is about 3000 mg/day. According to another embodiment, the therapeutic amount of N-acetyl-L-cysteine (NAC) is about 2800 mg/day. According to another embodiment, the therapeutic amount of N-acetyl-L-cysteine (NAC) is about 2600 mg/day. According to another embodiment, the therapeutic amount of N-acetyl-L-cysteine (NAC) is about 2400 mg/day. According to another embodiment, the therapeutic amount of N-acetyl-L-cysteine (NAC) is about 2200 mg/day. According to another embodiment, the therapeutic amount of N-acetyl-L-cysteine (NAC) is about 2000 mg/day. According to another embodiment, the therapeutic amount of N-acetyl-L-cysteine (NAC) is about 1800 mg/day. According to another embodiment, the therapeutic amount of N-acetyl-L-cysteine (NAC) is about 1600 mg/day. According to another embodiment, the therapeutic amount of N-acetyl-L-cysteine (NAC) is about 1400 mg/day. According to another embodiment, the therapeutic amount of N-acetyl-L-cysteine (NAC) is about 1200 mg/day. According to another embodiment, the therapeutic amount of N-acetyl-L-cysteine (NAC) is about 1000 mg/day. According to another embodiment, the therapeutic amount of N-acetyl-L-cysteine (NAC) is about 900 mg/day. According to another embodiment, the therapeutic amount of N-acetyl-L-cysteine (NAC) is about 800 mg/day. According to another embodiment, the therapeutic amount of N-acetyl-L-cysteine (NAC) is about 700 mg/day. According to another embodiment, the therapeutic amount of N-acetyl-L-cysteine (NAC) is about 600 mg/day. According to another embodiment, the therapeutic amount of N-acetyl-L-cysteine (NAC) is about 500 mg/day. According to another embodiment, the therapeutic amount of N-acetyl-L-cysteine (NAC) is about 450 mg/day. According to another embodiment, the therapeutic amount of N-acetyl-L-cysteine (NAC) is about 400 mg/day. According to another embodiment, the therapeutic amount of N-acetyl-L-cysteine (NAC) is about 350 mg/day. According to another embodiment, the therapeutic amount of N-acetyl-L-cysteine (NAC) is about 300 mg/day. According to another embodiment, the therapeutic amount of N-acetyl-L-cysteine (NAC) is about 250 mg/day. According to another embodiment, the therapeutic amount of N-acetyl-L-cysteine (NAC) is about 200 mg/day. According to another embodiment, the therapeutic amount of N-acetyl-L-cysteine (NAC) is about 150 mg/day. According to another embodiment, the therapeutic amount of N-acetyl-L-cysteine (NAC) is about 125 mg/day. According to another embodiment, the therapeutic amount of N-acetyl-L-cysteine (NAC) is about 100 mg/day. According to another embodiment, the therapeutic amount of N-acetyl-L-cysteine (NAC) is about 75 mg/day. According to another embodiment, the therapeutic amount of N-acetyl-L-cysteine (NAC) is about 50 mg/day. According to another embodiment, the therapeutic amount of N-acetyl-L-cysteine (NAC) is about 25 mg/day. According to another embodiment, the therapeutic amount of N-acetyl-L-cysteine (NAC) is about 10 mg/day. According to another embodiment, the therapeutic amount of N-acetyl-L-cysteine (NAC) is about 5 mg/day. According to another embodiment, the therapeutic amount of N-acetyl-L-cysteine (NAC) is about 1 mg/day.

According to some embodiments, the maximum daily adult dose of N-acetyl-L-cysteine (NAC) is from about 1 mg/kg body weight to about 100 mg/kg body weight. According to one embodiment, the maximum daily adult dose of N-acetyl-L-cysteine (NAC) is about 1 mg/kg body weight. According to another embodiment, the maximum daily adult dose of N-acetyl-L-cysteine (NAC) is about 2 mg/kg body weight. According to another embodiment, the maximum daily adult dose of N-acetyl-L-cysteine (NAC) is about 4 mg/kg body weight. According to another embodiment, the maximum daily adult dose of N-acetyl-L-cysteine (NAC) is about 6 mg/kg body weight. According to another embodiment, the maximum daily adult dose of N-acetyl-L-cysteine (NAC) is about 8 mg/kg body weight. According to another embodiment, the maximum daily adult dose of N-acetyl-L-cysteine (NAC) is about 10 mg/kg body weight. According to another embodiment, the maximum daily adult dose of N-acetyl-L-cysteine (NAC) is about 12 mg/kg body weight. According to another embodiment, the maximum daily adult dose of N-acetyl-L-cysteine (NAC) is about 14 mg/kg body weight. According to another embodiment, the maximum daily adult dose of N-acetyl-L-cysteine (NAC) is about 16 mg/kg body weight. According to another embodiment, the maximum daily adult dose of N-acetyl-L-cysteine (NAC) is about 18 mg/kg body weight. According to another embodiment, the maximum daily adult dose of N-acetyl-L-cysteine (NAC) is about 20 mg/kg body weight. According to another embodiment, the maximum daily adult dose of N-acetyl-L-cysteine (NAC) is about 22 mg/kg body weight. According to another embodiment, the maximum daily adult dose of N-acetyl-L-cysteine (NAC) is about 24 mg/kg body weight. According to another embodiment, the maximum daily adult dose of N-acetyl-L-cysteine (NAC) is about 26 mg/kg body weight. According to another embodiment, the maximum daily adult dose of N-acetyl-L-cysteine (NAC) is about 28 mg/kg body weight. According to another embodiment, the maximum daily adult dose of N-acetyl-L-cysteine (NAC) is about 30 mg/kg body weight. According to another embodiment, the maximum daily adult dose of N-acetyl-L-cysteine (NAC) is about 32 mg/kg body weight. According to another embodiment, the maximum daily adult dose of N-acetyl-L-cysteine (NAC) is about 34 mg/kg body weight. According to another embodiment, the maximum daily adult dose of N-acetyl-L-cysteine (NAC) is about 36 mg/kg body weight. According to another embodiment, the maximum daily adult dose of N-acetyl-L-cysteine (NAC) is about 38 mg/kg body weight. According to another embodiment, the maximum daily adult dose of N-acetyl-L-cysteine (NAC) is about 40 mg/kg body weight. According to another embodiment, the maximum daily adult dose of N-acetyl-L-cysteine (NAC) is about 42 mg/kg body weight. According to another embodiment, the maximum daily adult dose of N-acetyl-L-cysteine (NAC) is about 44 mg/kg body weight. According to another embodiment, the maximum daily adult dose of N-acetyl-L-cysteine (NAC) is about 46 mg/kg body weight. According to another embodiment, the maximum daily adult dose of N-acetyl-L-cysteine (NAC) is about 48 mg/kg body weight. According to another embodiment, the maximum daily adult dose of N-acetyl-L-cysteine (NAC) is about 50 mg/kg body weight. According to another embodiment, the maximum daily adult dose of N-acetyl-L-cysteine (NAC) is about 52 mg/kg body weight. According to another embodiment, the maximum daily adult dose of N-acetyl-L-cysteine (NAC) is about 54 mg/kg body weight. According to another embodiment, the maximum daily adult dose of N-acetyl-L-cysteine (NAC) is about 56 mg/kg body weight. According to another embodiment, the maximum daily adult dose of N-acetyl-L-cysteine (NAC) is about 58 mg/kg body weight. According to another embodiment, the maximum daily adult dose of N-acetyl-L-cysteine (NAC) is about 60 mg/kg body weight. According to another embodiment, the maximum daily adult dose of N-acetyl-L-cysteine (NAC) is about 62 mg/kg body weight. According to another embodiment, the maximum daily adult dose of N-acetyl-L-cysteine (NAC) is about 64 mg/kg body weight. According to another embodiment, the maximum daily adult dose of N-acetyl-L-cysteine (NAC) is about 66 mg/kg body weight. According to another embodiment, the maximum daily adult dose of N-acetyl-L-cysteine (NAC) is about 68 mg/kg body weight. According to another embodiment, the maximum daily adult dose of N-acetyl-L-cysteine (NAC) is about 70 mg/kg body weight. According to another embodiment, the maximum daily adult dose of N-acetyl-L-cysteine (NAC) is about 72 mg/kg body weight. According to another embodiment, the maximum daily adult dose of N-acetyl-L-cysteine (NAC) is about 74 mg/kg body weight. According to another embodiment, the maximum daily adult dose of N-acetyl-L-cysteine (NAC) is about 76 mg/kg body weight. According to another embodiment, the maximum daily adult dose of N-acetyl-L-cysteine (NAC) is about 78 mg/kg body weight. According to another embodiment, the maximum daily adult dose of N-acetyl-L-cysteine (NAC) is about 80 mg/kg body weight. According to another embodiment, the maximum daily adult dose of N-acetyl-L-cysteine (NAC) is about 82 mg/kg body weight. According to another embodiment, the maximum daily adult dose of N-acetyl-L-cysteine (NAC) is about 84 mg/kg body weight. According to another embodiment, the maximum daily adult dose of N-acetyl-L-cysteine (NAC) is about 86 mg/kg body weight. According to another embodiment, the maximum daily adult dose of N-acetyl-L-cysteine (NAC) is about 88 mg/kg body weight. According to another embodiment, the maximum daily adult dose of N-acetyl-L-cysteine (NAC) is about 90 mg/kg body weight. According to another embodiment, the maximum daily adult dose of N-acetyl-L-cysteine (NAC) is about 92 mg/kg body weight. According to another embodiment, the maximum daily adult dose of N-acetyl-L-cysteine (NAC) is about 94 mg/kg body weight. According to another embodiment, the maximum daily adult dose of N-acetyl-L-cysteine (NAC) is about 96 mg/kg body weight. According to another embodiment, the maximum daily adult dose of N-acetyl-L-cysteine (NAC) is about 98 mg/kg body weight. According to another embodiment, the maximum daily adult dose of N-acetyl-L-cysteine (NAC) is about 100 mg/kg body weight.

According to some embodiments, the maximum daily pediatric dose of N-acetyl-L-cysteine (NAC) is from about 0.1 mg/kg body weight to about 11 mg/kg body weight. According to one embodiment, the maximum daily pediatric dose of N-acetyl-L-cysteine (NAC) is about 0.1 mg/kg body weight. According to another embodiment, the maximum daily pediatric dose of N-acetyl-L-cysteine (NAC) is about 0.2 mg/kg body weight. According to another embodiment, the maximum daily pediatric dose of N-acetyl-L-cysteine (NAC) is about 0.4 mg/kg body weight. According to another embodiment, the maximum daily pediatric dose of N-acetyl-L-cysteine (NAC) is about 0.6 mg/kg body weight. According to another embodiment, the maximum daily pediatric dose of N-acetyl-L-cysteine (NAC) is about 0.8 mg/kg body weight. According to another embodiment, the maximum daily pediatric dose of N-acetyl-L-cysteine (NAC) is about 1.0 mg/kg body weight. According to another embodiment, the maximum daily pediatric dose of N-acetyl-L-cysteine (NAC) is about 1.2 mg/kg body weight. According to another embodiment, the maximum daily pediatric dose of N-acetyl-L-cysteine (NAC) is about 1.4 mg/kg body weight. According to another embodiment, the maximum daily pediatric dose of N-acetyl-L-cysteine (NAC) is about 1.6 mg/kg body weight. According to another embodiment, the maximum daily pediatric dose of N-acetyl-L-cysteine (NAC) is about 1.8 mg/kg body weight. According to another embodiment, the maximum daily pediatric dose of N-acetyl-L-cysteine (NAC) is about 2.0 mg/kg body weight. According to another embodiment, the maximum daily pediatric dose of N-acetyl-L-cysteine (NAC) is about 2.2 mg/kg body weight. According to another embodiment, the maximum daily pediatric dose of N-acetyl-L-cysteine (NAC) is about 2.4 mg/kg body weight. According to another embodiment, the maximum daily pediatric dose of N-acetyl-L-cysteine (NAC) is about 2.6 mg/kg body weight. According to another embodiment, the maximum daily pediatric dose of N-acetyl-L-cysteine (NAC) is about 2.8 mg/kg body weight. According to another embodiment, the maximum daily pediatric dose of N-acetyl-L-cysteine (NAC) is about 3.0 mg/kg body weight. According to another embodiment, the maximum daily pediatric dose of N-acetyl-L-cysteine (NAC) is about 3.2 mg/kg body weight. According to another embodiment, the maximum daily pediatric dose of N-acetyl-L-cysteine (NAC) is about 3.4 mg/kg body weight. According to another embodiment, the maximum daily pediatric dose of N-acetyl-L-cysteine (NAC) is about 3.6 mg/kg body weight. According to another embodiment, the maximum daily pediatric dose of N-acetyl-L-cysteine (NAC) is about 3.8 mg/kg body weight. According to another embodiment, the maximum daily pediatric dose of N-acetyl-L-cysteine (NAC) is about 4.0 mg/kg body weight. According to another embodiment, the maximum daily pediatric dose of N-acetyl-L-cysteine (NAC) is about 4.2 mg/kg body weight. According to another embodiment, the maximum daily pediatric dose of N-acetyl-L-cysteine (NAC) is about 4.4 mg/kg body weight. According to another embodiment, the maximum daily pediatric dose of N-acetyl-L-cysteine (NAC) is about 4.6 mg/kg body weight. According to another embodiment, the maximum daily pediatric dose of N-acetyl-L-cysteine (NAC) is about 4.8 mg/kg body weight. According to another embodiment, the maximum daily pediatric dose of N-acetyl-L-cysteine (NAC) is about 5.0 mg/kg body weight. According to another embodiment, the maximum daily pediatric dose of N-acetyl-L-cysteine (NAC) is about 5.2 mg/kg body weight. According to another embodiment, the maximum daily pediatric dose of N-acetyl-L-cysteine (NAC) is about 5.4 mg/kg body weight. According to another embodiment, the maximum daily pediatric dose of N-acetyl-L-cysteine (NAC) is about 5.6 mg/kg body weight. According to another embodiment, the maximum daily pediatric dose of N-acetyl-L-cysteine (NAC) is about 5.8 mg/kg body weight. According to another embodiment, the maximum daily pediatric dose of N-acetyl-L-cysteine (NAC) is about 6.0 mg/kg body weight. According to another embodiment, the maximum daily pediatric dose of N-acetyl-L-cysteine (NAC) is about 6.2 mg/kg body weight. According to another embodiment, the maximum daily pediatric dose of N-acetyl-L-cysteine (NAC) is about 6.4 mg/kg body weight. According to another embodiment, the maximum daily pediatric dose of N-acetyl-L-cysteine (NAC) is about 6.6 mg/kg body weight. According to another embodiment, the maximum daily pediatric dose of N-acetyl-L-cysteine (NAC) is about 6.8 mg/kg body weight. According to another embodiment, the maximum daily pediatric dose of N-acetyl-L-cysteine (NAC) is about 7.0 mg/kg body weight. According to another embodiment, the maximum daily pediatric dose of N-acetyl-L-cysteine (NAC) is about 7.2 mg/kg body weight. According to another embodiment, the maximum daily pediatric dose of N-acetyl-L-cysteine (NAC) is about 7.4 mg/kg body weight. According to another embodiment, the maximum daily pediatric dose of N-acetyl-L-cysteine (NAC) is about 7.6 mg/kg body weight. According to another embodiment, the maximum daily pediatric dose of N-acetyl-L-cysteine (NAC) is about 7.8 mg/kg body weight. According to another embodiment, the maximum daily pediatric dose of N-acetyl-L-cysteine (NAC) is about 8.0 mg/kg body weight. According to another embodiment, the maximum daily pediatric dose of N-acetyl-L-cysteine (NAC) is about 8.2 mg/kg body weight. According to another embodiment, the maximum daily pediatric dose of N-acetyl-L-cysteine (NAC) is about 8.4 mg/kg body weight. According to another embodiment, the maximum daily pediatric dose of N-acetyl-L-cysteine (NAC) is about 8.6 mg/kg body weight. According to another embodiment, the maximum daily pediatric dose of N-acetyl-L-cysteine (NAC) is about 8.8 mg/kg body weight. According to another embodiment, the maximum daily pediatric dose of N-acetyl-L-cysteine (NAC) is about 9.0 mg/kg body weight. According to another embodiment, the maximum daily pediatric dose of N-acetyl-L-cysteine (NAC) is about 9.2 mg/kg body weight. According to another embodiment, the maximum daily pediatric dose of N-acetyl-L-cysteine (NAC) is about 9.4 mg/kg body weight. According to another embodiment, the maximum daily pediatric dose of N-acetyl-L-cysteine (NAC) is about 9.5 mg/kg body weight. According to another embodiment, the maximum daily pediatric dose of N-acetyl-L-cysteine (NAC) is about 9.6 mg/kg body weight. According to another embodiment, the maximum daily pediatric dose of N-acetyl-L-cysteine (NAC) is about 9.7 mg/kg body weight. According to another embodiment, the maximum daily pediatric dose of N-acetyl-L-cysteine (NAC) is about 9.8 mg/kg body weight. According to another embodiment, the maximum daily pediatric dose of N-acetyl-L-cysteine (NAC) is about 9.9 mg/kg body weight. According to another embodiment, the maximum daily pediatric dose of N-acetyl-L-cysteine (NAC) is about 10 mg/kg body weight. According to another embodiment, the maximum daily pediatric dose of N-acetyl-L-cysteine (NAC) is about 10.1 mg/kg body weight. According to another embodiment, the maximum daily pediatric dose of N-acetyl-L-cysteine (NAC) is about 10.2 mg/kg body weight. According to another embodiment, the maximum daily pediatric dose of N-acetyl-L-cysteine (NAC) is about 10.3 mg/kg body weight. According to another embodiment, the maximum daily pediatric dose of N-acetyl-L-cysteine (NAC) is about 10.4 mg/kg body weight. According to another embodiment, the maximum daily pediatric dose of N-acetyl-L-cysteine (NAC) is about 10.5 mg/kg body weight. According to another embodiment, the maximum daily pediatric dose of N-acetyl-L-cysteine (NAC) is about 10.6 mg/kg body weight. According to another embodiment, the maximum daily pediatric dose of N-acetyl-L-cysteine (NAC) is about 10.7 mg/kg body weight. According to another embodiment, the maximum daily pediatric dose of N-acetyl-L-cysteine (NAC) is about 10.8 mg/kg body weight. According to another embodiment, the maximum daily pediatric dose of N-acetyl-L-cysteine (NAC) is about 10.9 mg/kg body weight. According to another embodiment, the maximum daily pediatric dose of N-acetyl-L-cysteine (NAC) is about 11 mg/kg body weight.

According to some embodiments, a compound of Formula I, or a combination thereof may be provided according to the present invention in any of a variety of useful forms, for example as pharmaceutically acceptable salts, as particular crystal forms, etc. According to some embodiments, a prodrug of one or more compounds of the present invention are provided. Various forms of prodrug are known in the art, for example as discussed in Bundgaard (ed.), Design of Prodrugs, Elsevier (1985); Widder et al. (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975), the entire disclosure of each of which is incorporated herein by reference.

According to one embodiment, the therapeutic amount of the N-acetyl-L-cysteine (NAC) is effective to reduce lupus disease activity in the subject compared to an untreated control. According to some such embodiments, the lupus disease activity is measured by a disease activity score selected from the group consisting of systemic lupus erythematosus disease activity index (SLEDAI) score, British Isles Lupus Assessment Group (BILAG) score, fatigue assessment scale (FAS) score, or a combination thereof.

According to some such embodiments, the therapeutic amount of N-acetyl-L-cysteine (NAC) is effective to reduce systemic lupus erythematosus disease activity index (SLEDAI) compared to an untreated control at least after at least 7 days of the administration, after at least 14 days of the administration, after at least 1 month of the administration, after at least 2 months of the administration, after at least 3 months of the administration, or after at least 4 months of the administration. According to some such embodiments, the therapeutic amount of N-acetyl-L-cysteine (NAC) is effective to reduce systemic lupus erythematosus disease activity index (SLEDAI) by at least 1 point compared to an untreated control after at least 1 month of the administration. According to some such embodiments, the therapeutic amount of N-acetyl-L-cysteine (NAC) is effective to reduce systemic lupus erythematosus disease activity index (SLEDAI) by at least 1.1 points compared to an untreated control after at least 1 month of the administration. According to some such embodiments, the therapeutic amount of N-acetyl-L-cysteine (NAC) is effective to reduce systemic lupus erythematosus disease activity index (SLEDAI) by at least 1.5 points compared to an untreated control after at least 1 month of the administration. According to some such embodiments, the therapeutic amount of N-acetyl-L-cysteine (NAC) is effective to reduce systemic lupus erythematosus disease activity index (SLEDAI) by at least 2.0 points compared to an untreated control after at least 1 month of the administration. According to some such embodiments, the therapeutic amount of N-acetyl-L-cysteine (NAC) is effective to reduce systemic lupus erythematosus disease activity index (SLEDAI) by at least 2.1 points compared to an untreated control after at least 1 month of the administration. According to some such embodiments, the therapeutic amount of N-acetyl-L-cysteine (NAC) is effective to reduce systemic lupus erythematosus disease activity index (SLEDAI) by at least 2.2 points compared to an untreated control after at least 1 month of the administration. According to some such embodiments, the therapeutic amount of N-acetyl-L-cysteine (NAC) is effective to reduce systemic lupus erythematosus disease activity index (SLEDAI) by at least 2.3 points compared to an untreated control after at least 1 month of the administration.

According to some such embodiments, the therapeutic amount of N-acetyl-L-cysteine (NAC) is effective to reduce British Isles Lupus Assessment Group (BILAG) score compared to an untreated control after at least 7 days of the administration, at least after 14 days of the administration, at least after 1 month of the administration, at least after 2 months of the administration, at least after 3 months of the administration, or at least after 4 months of the administration. According to some such embodiments, the therapeutic amount of N-acetyl-L-cysteine (NAC) is effective to reduce British Isles Lupus Assessment Group (BILAG) score by at least 1 point compared to an untreated control after at least 1 month of the administration. According to some such embodiments, the therapeutic amount of N-acetyl-L-cysteine (NAC) is effective to reduce British Isles Lupus Assessment Group (BILAG) score by at least 2.0 points compared to an untreated control after at least 1 month of the administration. According to some such embodiments, the therapeutic amount of N-acetyl-L-cysteine (NAC) is effective to reduce British Isles Lupus Assessment Group (BILAG) score by at least 2.5 points compared to an untreated control after at least 1 month of the administration. According to some such embodiments, the therapeutic amount of N-acetyl-L-cysteine (NAC) is effective to reduce British Isles Lupus Assessment Group (BILAG) score by at least 3.0 points compared to an untreated control after at least 1 month of the administration. According to some such embodiments, the therapeutic amount of N-acetyl-L-cysteine (NAC) is effective to reduce British Isles Lupus Assessment Group (BILAG) score by at least 3.5 points compared to an untreated control after at least 1 month of the administration. According to some such embodiments, the therapeutic amount of N-acetyl-L-cysteine (NAC) is effective to reduce British Isles Lupus Assessment Group (BILAG) score by at least 4.0 points compared to an untreated control after at least 1 month of the administration. According to some such embodiments, the therapeutic amount of N-acetyl-L-cysteine (NAC) is effective to reduce British Isles Lupus Assessment Group (BILAG) score by at least 4.5 points compared to an untreated control after at least 1 month of the administration. According to some such embodiments, the therapeutic amount of N-acetyl-L-cysteine (NAC) is effective to reduce British Isles Lupus Assessment Group (BILAG) score by at least 5.0 points compared to an untreated control after at least 1 month of the administration.

According to some such embodiments, the therapeutic amount of N-acetyl-L-cysteine (NAC) is effective to reduce fatigue assessment scale (FAS) score compared to an untreated control after at least 7 days of the administration, at least after 14 days of the administration, at least after 1 month of the administration, at least after 2 months of the administration, at least after 3 months of the administration, or at least after 4 months of the administration. According to some such embodiments, the therapeutic amount of N-acetyl-L-cysteine (NAC) is effective to reduce fatigue assessment scale (FAS) score by at least 1 point compared to an untreated control after at least 1 month of the administration. According to some such embodiments, the therapeutic amount of N-acetyl-L-cysteine (NAC) is effective to reduce fatigue assessment scale (FAS) score by at least 2.0 points compared to an untreated control after at least 1 month of the administration. According to some such embodiments, the therapeutic amount of N-acetyl-L-cysteine (NAC) is effective to reduce fatigue assessment scale (FAS) score by at least 2.5 points compared to an untreated control after at least 1 month of the administration. According to some such embodiments, the therapeutic amount of N-acetyl-L-cysteine (NAC) is effective to reduce fatigue assessment scale (FAS) score by at least 3.0 points compared to an untreated control after at least 1 month of the administration. According to some such embodiments, the therapeutic amount of N-acetyl-L-cysteine (NAC) is effective to reduce fatigue assessment scale (FAS) score by at least 3.5 points compared to an untreated control after at least 1 month of the administration. According to some such embodiments, the therapeutic amount of N-acetyl-L-cysteine (NAC) is effective to reduce fatigue assessment scale (FAS) score by at least 4.0 points compared to an untreated control after at least 1 month of the administration. According to some such embodiments, the therapeutic amount of N-acetyl-L-cysteine (NAC) is effective to reduce fatigue assessment scale (FAS) score by at least 4.5 points compared to an untreated control after at least 1 month of the administration. According to some such embodiments, the therapeutic amount of N-acetyl-L-cysteine (NAC) is effective to reduce fatigue assessment scale (FAS) score by at least 5.0 points compared to an untreated control after at least 1 month of the administration.

According to one embodiment, the therapeutic amount of N-acetyl-L-cysteine (NAC) is effective to increase mitochondrial mass of T cells of the subject compared to an untreated control. According to some embodiments, the therapeutic amount of N-acetyl-L-cysteine (NAC) is effective to increase mitochondrial mass of T cells of the subject compared to compared to an untreated control after at least 7 days of the administration, after at least 14 days of the administration, after at least 1 month of the administration, after at least 2 months of the administration, after at least 3 months of the administration, or after at least 4 months of the administration.

According to another embodiment, the therapeutic amount of N-acetyl-L-cysteine (NAC) is effective to increase mitochondrial membrane potential in double negative (DN) T cells of the subject compared to an untreated control. According to some embodiments, the therapeutic amount of N-acetyl-L-cysteine (NAC) is effective to increase mitochondrial membrane potential in double negative (DN) T cells of the subject compared to an untreated control after at least 7 days of the administration, after at least 14 days of the administration, after at least 1 month of the administration, after at least 2 months of the administration, after at least 3 months of the administration, or after at least 4 months of the administration.

According to another embodiment, the therapeutic amount of N-acetyl-L-cysteine (NAC) is effective to increase a level of a reactive oxygen intermediate (ROI) in double negative (DN) T cells of the subject compared to an untreated control. According to one embodiment, the oxygen intermediate (ROI) is hydrogen peroxide. According to some embodiments, the therapeutic amount of N-acetyl-L-cysteine (NAC) is effective to increase the level of a reactive oxygen intermediate (ROI) in double negative (DN) T cells of the subject compared to an untreated control after at least 7 days of the administration, after at least 14 days of the administration, after at least 1 month of the administration, after at least 2 months of the administration, after at least 3 months of the administration, or after at least 4 months of the administration.

According to another embodiment, the therapeutic amount of N-acetyl-L-cysteine (NAC) is effective to increase the spontaneous apoptotic rate of double negative (DN) T cells of the subject compared to an untreated control. According to some embodiments, the therapeutic amount of N-acetyl-L-cysteine (NAC) is effective to increase the spontaneous apoptotic rate of double negative (DN) T cells of the subject compared to an untreated control after at least 7 days of the administration, after at least 14 days of the administration, after at least 1 month of the administration, after at least 2 months of the administration, after at least 3 months of the administration, or after at least 4 months of the administration.

According to another embodiment, the therapeutic amount of N-acetyl-L-cysteine (NAC) is effective to increase the activation-induced apoptotic rate of double negative (DN) T cells of the subject compared to an untreated control. According to some embodiments, the therapeutic amount of N-acetyl-L-cysteine (NAC) is effective to increase the activation-induced apoptotic rate of double negative (DN) T cells of the subject compared to an untreated control after at least 7 days of the administration, after at least 14 days of the administration, after at least 1 month of the administration, after at least 2 months of the administration, after at least 3 months of the administration, or after at least 4 months of the administration.

According to another embodiment, the therapeutic amount of N-acetyl-L-cysteine (NAC) is effective to decrease activity of mammalian target of rapamycin (mTOR) compared to an untreated control. According to some embodiments, the therapeutic amount of N-acetyl-L-cysteine (NAC) is effective to decrease activity of mammalian target of rapamycin (mTOR) compared to an untreated control after at least 7 days of the administration, after at least 14 days of the administration, after at least 1 month of the administration, after at least 2 months of the administration, after at least 3 months of the administration, or after at least 4 months of the administration. According to some such embodiments, the therapeutic amount of N-acetyl-L-cysteine (NAC) is effective to decrease phosphorylated ribosomal protein S6 (p-RPS6^{high}) cells in double negative (DN) T cells of the subject by at least 2-fold compared to an untreated control.

According to another embodiment, the therapeutic amount of N-acetyl-L-cysteine (NAC) is effective to increase number of FoxP3+CD8+CD25+ T cells compared to an untreated control. According to some embodiments, the therapeutic amount of N-acetyl-L-cysteine (NAC) is effective to increase number of FoxP3+CD8+CD25+ T cells compared to an untreated control after at least 7 days of the administration, after at least 14 days of the administration, after at least 1 month of the administration, after at least 2 months of the administration, after at least 3 months of the administration, or after at least 4 months of the administration.

According to another embodiment, the therapeutic amount of N-acetyl-L-cysteine (NAC) is effective to reduce a cognitive/inattentive component of attention deficit and hyperactivity (ADHD) self-report scale (ASRS) compared to an untreated control. According to another embodiment, the therapeutic amount of N-acetyl-L-cysteine (NAC) is effective to reduce a cognitive/inattentive component of attention deficit and hyperactivity (ADHD) self-report scale (ASRS) compared to an untreated control after at least 7 days of the administration, after at least 14 days of the administration, after at least 1 month of the administration, after at least 2 months of the administration, after at least 3 months of the administration, or after at least 4 months of the administration.

### Combination Therapy

According to another embodiment, the compositions of the present invention can further comprise one or more additional compatible active ingredients. “Compatible” as used herein means that the components of such a composition are capable of being combined with each other in a manner such that there is no interaction that would substantially reduce the efficacy of the composition under ordinary use conditions. According to another embodiment, the composition further comprises at least one additional therapeutic agent. According to another embodiment, the additional therapeutic agent is of a therapeutic amount effective to exert an additive effect in treating or alleviating one or more symptoms of the lupus condition. According to another embodiment, the additional therapeutic agent is of a therapeutic amount effective to exert a synergistic effect in treating or alleviating one or more symptoms of the lupus condition.

According to some embodiments, the additional therapeutic agent is selected from a group consisting of a non-steroidal anti-inflammatory agent, an antimalarial agent, a corticosteroid, a cytotoxic agent, an immunosuppressive agent, a biologic, or a combination thereof. According to one embodiment, the additional therapeutic agent is a non-steroidal anti-inflammatory agent. Exemplary non-steroidal anti-inflammatory agents include but are not limited to salicylate derivates (e.g. aspirin, arthopan), celecoxib (Celebrix®), diclofenac (Cataflam®, Voltaren®), etodolac (Lodine®), fenprofen (Nalfon®), flurbiprofen (Ansaid®), ibuprofen (Motrin®, Advil®, Nuprin®), ketoprofen (Orudis®, Actron®), meclofamate (Meclomen®), meloxicam (Mobic®), nabumetone (Relafen®), naproxen (Aleve®, Naprosyn®, Anaprox®), oxaprozin (Daypro®), piroxicam (Feldene®), rofecoxib (Vioxx®), sulindac (Clinoril®), tolmetin (Tolectin®) and acetaminophen (Tylenol®). According to another embodiment, the additional therapeutic agent is an antimalarial agent. Exemplary antimalarial agents include but are not limited to hydroxycloroquine (Plauenil®), chloroquine (Aralen®), quinicrine (Atabrine®). According to another embodiment, the additional therapeutic agent is a corticosteroid. Exemplary corticocorticosteroids include but are not limited to topical creams or ointments such as clobetasol (Temovate®), halobetasol (Ultravate®), hydrocortisone (Cortel®, Cortaid®), triamcinolone (Aristocort®, Kenalog®), betamethasone (Valisone®, Diprosone®), fluocinolone (Synalar®), fluocinonide (Lidex®); tablets such as prednisone (Deltasone®), prednisolone (Prelone®), ethylprednisone (Medrol®); and intravenous formulations such as methylprednisone (Solu-Medrol®), hydrocortisone (Solu-Cortel®). According to another embodiment, the additional therapeutic agent is a cytotoxic agent. Exemplary cytotoxic agents include but are not limited to azathioprine (Imuran®), cyclophosphamide (Cytoxan®), mycophenolate mofetil (Cellcept®), cyclosporine A (Sandimmune®, Neoral®), methotrexate (Rhematrex®), chlorambucil (Leukeran®). According to another embodiment, the additional therapeutic agent is an immunosuppressive agent. Exemplary immunosuppressive agents include but are not limited to azathioprine (Imuran®), cyclophosphamide (Cytoxan®), mycophenolate mofetil (Cellcept®), cyclosporine A (Sandimmune®, Neoral®), methotrexate (Rhematrex®), chlorambucil (Leukeran®). According to another embodiment, the additional therapeutic agent is a biologic. Exemplary biologics include but are not limited to a B-cell target biologic (Ezpratuzumab®, Rituximab®, Belimumab®), a T cell target biologic (Abatcept, rapamycin), a spleen tyrosine kinase antagonist (R788), a tumor necrosis factor (TNF) antagonist, an interferon antagonist, an interleukin-6-receptor antagonist.

For any therapeutic agent described herein the therapeutically effective amount may be initially determined from preliminary in vitro studies and/or animal models. A therapeutically effective dose may also be determined from human data for the lupus condition. The applied dose may be adjusted based on the relative bioavailability and potency of the administered compound. Adjusting the dose to achieve maximal efficacy based on the methods described above and other methods as are well-known in the art is well within the capabilities of the ordinarily skilled artisan.

General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

The term “therapeutic window” refers to a concentration range that provides therapeutic efficacy without unacceptable toxicity. Following administration of a dose of a drug, its effects usually show a characteristic temporal pattern. A lag period is present before the drug concentration exceeds the minimum effective concentration (“MEC”) for the desired effect. Following onset of the response, the intensity of the effect increases as the drug continues to be absorbed and distributed. This reaches a peak, after which drug elimination results in a decline in the effect's intensity that disappears when the drug concentration falls back below the MEC. Accordingly, the duration of a drug's action is determined by the time period over which concentrations exceed the MEC. The therapeutic goal is to obtain and maintain concentrations within the therapeutic window for the desired response with a minimum of toxicity. Drug response below the MEC for the desired effect will be subtherapeutic, whereas for an adverse effect, the probability of toxicity will increase above the MEC. Increasing or decreasing drug dosage shifts the response curve up or down the intensity scale and is used to modulate the drug's effect. Increasing the dose also prolongs a drug's duration of action but at the risk of increasing the likelihood of adverse effects. Accordingly, unless the drug is nontoxic, increasing the dose is not a useful strategy for extending a drug's duration of action.

Instead, another dose of drug should be given to maintain concentrations within the therapeutic window. In general, the lower limit of the therapeutic range of a drug appears to be approximately equal to the drug concentration that produces about half of the greatest possible therapeutic effect, and the upper limit of the therapeutic range is such that no more than about 5% to about 10% of patients will experience a toxic effect. These figures can be highly variable, and some patients may benefit greatly from drug concentrations that exceed the therapeutic range, while others may suffer significant toxicity at much lower values. The therapeutic goal is to maintain steady-state drug levels within the therapeutic window. For most drugs, the actual concentrations associated with this desired range are not and need not be known, and it is sufficient to understand that efficacy and toxicity are generally concentration-dependent, and how drug dosage and frequency of administration affect the drug level. For a small number of drugs where there is a small (two- to three-fold) difference between concentrations resulting in efficacy and toxicity, a plasma-concentration range associated with effective therapy has been defined.

In this case, a target level strategy is reasonable, wherein a desired target steady-state concentration of the drug (usually in plasma) associated with efficacy and minimal toxicity is chosen, and a dosage is computed that is expected to achieve this value. Drug concentrations subsequently are measured and dosage is adjusted if necessary to approximate the target more closely.

In most clinical situations, drugs are administered in a series of repetitive doses or as a continuous infusion to maintain a steady-state concentration of drug associated with the therapeutic window. To maintain the chosen steady-state or target concentration (“maintenance dose”), the rate of drug administration is adjusted such that the rate of input equals the rate of loss. If the clinician chooses the desired concentration of drug in plasma and knows the clearance and bioavailability for that drug in a particular patient, the appropriate dose and dosing interval can be calculated.

The formulations may be presented conveniently in unit dosage form and may be prepared by any of the methods well known in the art of pharmacy. All methods include the step of bringing into association N-acetyl-L-cysteine (NAC), or a pharmaceutically acceptable salt or solvate thereof (“active compound”) with the carrier which constitutes one or more accessory agents. In general, the formulations are prepared by uniformly and intimately bringing into association the active agent with liquid carriers or finely divided solid carriers or both and then, if necessary, shaping the product into the desired formulation.

According to another embodiment, the composition is a pharmaceutical composition.

### NAC Packaging Stability

Over-the-counter NAC can be produced variably and packaged. Because production and packaging methods generally do not guard against oxidation, NAC can be significantly contaminated with bioactive oxidation products. These may be particularly important in view of data indicating that the oxidized form of NAC has effects counter to those reported for NAC and is bioactive at doses roughly 10-100 fold less than NAC (see Samstrand et al (1999) J. Pharmacol. Exp. Ther. 288: 1174-84).

The distribution of the oxidation states of NAC as a thiol and disulfide depends on the oxidation/reduction potential. The half-cell potential obtained for the NAC thiol/disulfide pair is about +63 mV, indicative of its strong reducing activity among natural compounds (see Noszal et al. (2000) J. Med. Chem. 43:2176-2182).

It therefore is highly desirable that the composition of the described invention is prepared and stored so that oxidation of the reduced form of NAC is minimized. When in solution, NAC containing formulations may be stored in a brown bottle that is vacuum sealed. In some embodiments, storage is in a cool dark environment. According to some embodiments, NAC containing formulations in solid form are blister packed under gas.

According to some embodiments, the composition is formulated as a tablet, wherein the tablet comprises at least one anti-oxidant agent. According to some such embodiments, the tablet is uncoated. According to some such embodiments, the tablet is coated with a coating that acts to, for example, limit oxygen transfer or photolability. According to another embodiment, the composition further comprises stabilizing agents. Stabilizing agents may include, but are not limited to, antioxidant agents. Such agents may act to, for example, but not limited to, inhibit oxygen transfer or photolability.

The determination of reduced and oxidized species present in a sample may be determined by various methods known in the art, for example, with capillary electrophoresis, HPLC, etc. as described by Chassaing et al., J. Chromatogr. B Biomed. Sci. Appl. 735(2): 219-227 (1999), the entire disclosure of which is incorporated herein by reference.

### Administration

According to some embodiments, the administering step (b) comprises administering the composition orally, topically, parenterally, buccally, sublingually, by inhalation, or rectally. According to one embodiment, the administering step (b) comprises administering the composition orally. According to another embodiment, the administering step (b) comprises administering the composition topically. According to another embodiment, the administering step (b) comprises administering the composition parenterally. According to another embodiment, the administering step (b) comprises administering the composition buccally. According to another embodiment, the administering step (b) comprises administering the composition sublingually. According to another embodiment, the administering step (b) comprises administering the composition by inhalation. According to another embodiment, the administering step (b) comprises administering the composition rectally.

According to some embodiments, the composition is in the form of a tablet, a pill, a gel, an injectable solution, an aerosol, a troche, a lozenge, an aqueous suspension, an oily suspension, a dispersible powder, a granule, a bead, an emulsion, an implant, a cream, a patch, a capsule, a syrup, a suppository or an insert. According to one embodiment, the composition is in the form of a tablet. According to another embodiment, the composition is in the form of a pill. According to another embodiment, the composition is in the form of a gel. According to another embodiment, the composition is in the form of an injectable solution. According to another embodiment, the composition is in the form of an aerosol. According to another embodiment, the composition is in the form of a troche. According to another embodiment, the composition is in the form of a lozenge. According to another embodiment, the composition is in the form of an aqueous suspension. According to another embodiment, the composition is in the form an oily suspension. According to another embodiment, the composition is in the form of a dispersible powder. According to another embodiment, the composition is in the form of a granule. According to another embodiment, the composition is in the form of a bead. According to another embodiment, the composition is in the form of an emulsion. According to another embodiment, the composition is in the form of an implant. According to another embodiment, the composition is in the form of a cream. According to another embodiment, the composition is in the form of a patch. According to another embodiment, the composition is in the form of a capsule. According to another embodiment, the composition is in the form of a syrup. According to another embodiment, the composition is in the form of a suppository. According to another embodiment, the composition is in the form of an insert.

The compositions of the described invention can be administered orally, topically, parenterally, buccally, sublingually, by inhalation or insufflation (either through the mouth or through the nose), rectally, or by any means known to the skilled artisan. According to some embodiments, the composition of the described invention is a liquid solution, a suspension, an emulsion, a tablet, a pill, a capsule, a sustained release formulation, a delayed release formulation, a powder, or a suppository. The composition can be formulated with traditional binders and carriers such as triglycerides.

The composition can be administered in pharmaceutically acceptable solutions, which may routinely contain pharmaceutically acceptable concentrations of salt, buffering agents, preservatives, compatible carriers, adjuvants, and optionally other therapeutic agents.

Oral Administration

The compositions of the described invention may be in a form suitable for oral use, for example, as tablets, troches, lozenges, aqueous or oily suspensions, dispersible powders or granules, emulsions, hard or soft capsules or syrups or elixirs. For oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like.

Moreover, when desired or needed, suitable binders, lubricants, disintegrating agents and coloring agents also may be incorporated in the mixture. Powders and tablets may be comprised of from about 5 to about 95 percent inventive composition. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum and the like.

Compositions intended for oral use can be prepared according to any known method, and such compositions may contain one or more agents selected from the group consisting of sweetening agents, flavoring agents, coloring agents, and preserving agents in order to provide pharmaceutically elegant and palatable preparations.

Tablets may contain the active ingredient(s) in admixture with non-toxic pharmaceutically-acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example, magnesium stearate, stearic acid or talc. The tablets may be uncoated or they may be coated by known techniques, for example, to delay disintegration and absorption in the gastrointestinal tract and thereby provide a sustained action over a longer period, to protect the composition from oxidation or photodegradation; or for controlled release. For example, a time delay material such as glyceryl monostearate or glyceryl distearate can be employed.

Compositions of the described invention also may be formulated for oral use as hard gelatin capsules, where the active ingredient(s) is(are) mixed with an inert solid diluent, for example, calcium carbonate, calcium phosphate or kaolin, or soft gelatin capsules wherein the active ingredient(s) is (are) mixed with water or an oil medium, for example, peanut oil, liquid paraffin, or olive oil.

Liquid form preparations include solutions, suspensions and emulsions wherein the active ingredient(s) is (are) in admixture with excipients suitable for the manufacture of aqueous suspensions and emulsions. Such excipients are suspending agents, for example, sodium carboxymethylcellulose, methylcellulose, hydroxypropylmethylcellulose, sodium alginate, polyvinylpyrrolidone, gum tragacanth, and gum acacia; dispersing or wetting agents may be a naturally-occurring phosphatide such as lecithin, or condensation products of an alkylene oxide with fatty acids, for example, polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. As an example may be mentioned water or water-propylene glycol solutions for parenteral injections or addition of one or more coloring agents, one or more flavoring agents, and one or more sweetening agents, such as sucrose or saccharin and pacifiers for oral solutions, suspensions and emulsions.

Compositions of the described invention may be formulated as oily suspensions by suspending the active ingredient in a vegetable oil, for example arachis oil, olive oil, sesame oil or coconut oil, or in a mineral oil, such as liquid paraffin. The oily suspensions may contain a thickening agent, for example, beeswax, hard paraffin or cetyl alcohol. Sweetening agents, such as those set forth above, and flavoring agents may be added to provide a palatable oral preparation. These compositions can be preserved by the addition of an antioxidant such as ascorbic acid.

Compositions of the described invention may be formulated in the form of dispersible powders and granules suitable for preparation of an aqueous suspension by the addition of water. The active ingredient in such powders and granules is provided in admixture with a dispersing or wetting agent, suspending agent, and one or more preservatives. Suitable dispersing or wetting agents and suspending agents are exemplified by those already mentioned above. Additional excipients, or example, sweetening, flavoring and coloring agents also can be present.

The compositions of the invention also may be in the form of an emulsion. An emulsion is a two-phase system prepared by combining two immiscible liquid carriers, one of which is disbursed uniformly throughout the other and consists of globules that have diameters equal to or greater than those of the largest colloidal particles. The globule size is critical and must be such that the system achieves maximum stability. Usually, separation of the two phases will not occur unless a third substance, an emulsifying agent, is incorporated. Thus, a basic emulsion contains at least three components, the two immiscible liquid carriers and the emulsifying agent, as well as the active ingredient. Most emulsions incorporate an aqueous phase into a non-aqueous phase (or vice versa). However, it is possible to prepare emulsions that are basically non-aqueous, for example, anionic and cationic surfactants of the non-aqueous immiscible system glycerin and olive oil. Thus, the compositions of the invention may be in the form of an oil-in-water emulsion. The oily phase can be a vegetable oil, for example, olive oil or arachis oil, or a mineral oil, for example a liquid paraffin, or a mixture thereof. Suitable emulsifying agents may be naturally-occurring gums, for example, gum acacia or gum tragacanth, naturally-occurring phosphatides, for example soy bean, lecithin, and esters or partial esters derived from fatty acids and hexitol anhydrides, for example sorbitan monooleate, and condensation products of the partial esters with ethylene oxide, for example, polyoxyethylene sorbitan monooleate. The emulsions also may contain sweetening and flavoring agents.

The compositions of the invention also may be formulated as syrups and elixirs. Syrups and elixirs may be formulated with sweetening agents, for example, glycerol, propylene glycol, sorbitol or sucrose. Such formulations also may contain a demulcent, a preservative, and flavoring and coloring agents. Demulcents are protective agents employed primarily to alleviate irritation, particularly mucous membranes or abraded tissues. A number of chemical substances possess demulcent properties. These substances include the alginates, mucilages, gums, dextrins, starches, certain sugars, and polymeric polyhydric glycols. Others include acacia, agar, benzoin, carbomer, gelatin, glycerin, hydroxyethyl cellulose, hydroxypropyl cellulose, hydroxypropyl methylcellulose, propylene glycol, sodium alginate, tragacanth, hydrogels and the like.

For buccal administration, the compositions of the described invention may take the form of tablets or lozenges formulated in a conventional manner.

There are three general methods of tablet preparation: the wet-granulation method; the dry-granulation method; and direct compression. The method of preparation and the added ingredients are selected to give the tablet formulation the desirable physical characteristics allowing the rapid compression of tablets. After compression, the tablets must have a number of additional attributes such as appearance, hardness, disintegration ability, appropriate dissolution characteristics, and uniformity, which also are influenced both by the method of preparation and by the added materials present in the formulation.

According to another embodiment, the tablet is a compressed tablet (CT). Compressed tablets are solid dosage forms formed with pressure and contain no special coating. Generally, they are made from powdered, crystalline, or granular materials, alone or in combination with binders, disintegrants, controlled-release polymers, lubricants, diluents and colorants.

According to another embodiment, the tablet is a sugar-coated tablet. These are compressed tablets containing a sugar coating. Such coatings may be colored and are beneficial in covering up drug substances possessing objectionable tastes or odors and in protecting materials sensitive to oxidation.

According to another embodiment, the tablet is a film-coated tablet. These Compressed tablets are covered with a thin layer or film of a water-soluble material. Numerous polymeric substances with film-forming properties may be used.

According to another embodiment, the tablet is an enteric-coated tablet. These Compressed tablets are coated with substances that resist solution in gastric fluid but disintegrate in the intestine.

According to another embodiment, the tablet is a multiple compressed tablet. These tablets are made by more than one compression cycle. Layered tablets are prepared by compressing additional tablet granulation on a previously compressed granulation. The operation may be repeated to produce multilayered tablets of two or three layers. Press-coated tablets (dry-coated) are prepared by feeding previously compressed tablets into a special tableting machine and compressing another granulation layer around the preformed tablets.

According to another embodiment, the tablet is a controlled-release tablet. Compressed tablets can be formulated to release the drug slowly over a prolonged period of time. Hence, these dosage forms have been referred to as prolonged-release or sustained-release dosage forms.

According to another embodiment, the tablet is a tablet for solution. These Compressed tablets may be used to prepare solutions or to impart given characteristics to solutions.

According to some such embodiments, the tablet is an effervescent tablet. In addition to the drug, these tablets contain sodium bicarbonate and an organic acid such as tartaric acid or citric acid. In the presence of water, these additives react, liberating carbon dioxide that acts as a disintegrator and produce effervescence.

According to another embodiment, the tablet is a buccal and or sublingual tablet. These are small, flat, oval tablets intended for buccal administration and that by inserting into the buccal pouch may dissolve or erode slowly.

According to another embodiment, the tablet is a molded tablet or tablet triturate.

According to some embodiments, the tablet comprises a compressed core comprising at least one component of the described formulation; and a membrane forming composition. Formulations utilizing membrane forming compositions are known to those of skill in the art (see, for example, Remington's Pharmaceutical Sciences, 20th Ed., 2000). Such membrane forming compositions may include, for example, a polymer, such as, but not limited to, cellulose ester, cellulose ether, and cellulose ester-ether polymers, an amphiphilic triblock copolymer surfactant, such as ethylene oxide-propylene oxideethylene oxide, and a solvent, such as acetone, which forms a membrane over the core. The compressed core may contain a bi-layer core including a drug layer and a push layer.

Non-Oral Administration

The term “non-oral administration” represents any method of administration in which a composition is not provided in a solid or liquid oral dosage form, wherein such solid or liquid oral dosage form is traditionally intended to substantially release and or deliver the drug in the gastrointestinal tract beyond the mouth and/or buccal cavity. Such solid dosage forms include conventional tablets, capsules, caplets, etc., which do not substantially release the drug in the mouth or in the oral cavity. It is appreciated that many oral liquid dosage forms such as solutions, suspensions, emulsions, etc., and some oral solid dosage forms may release some of the drug in the mouth or in the oral cavity during the swallowing of these formulations. However, due to their very short transit time through the mouth and the oral cavities, the release of drug from these formulations in the mouth or the oral cavity is considered de minimus or insubstantial. Accordingly, it is understood that the term “non-oral” includes parenteral, transdermal, inhalation, implant, and vaginal or rectal formulations and administrations. Further, implant formulations are to be included in the term “non-oral,” regardless of the physical location of implantation. Particularly, implantation formulations are known which are specifically designed for implantation and retention in the gastrointestinal tract. Such implants are also considered to be non-oral delivery formulations, and therefore are encompassed by the term “non-oral.”

Rectal Administration

The compositions of the described invention may be in the form of suppositories for rectal administration of the composition, such as for treating pediatric fever. The terms “Rectal” or “rectally” as used herein refer to introduction into the body through the rectum where absorption occurs through the walls of the rectum. These compositions can be prepared by mixing the drug with a suitable nonirritating excipient such as cocoa butter and polyethylene glycols which are solid at ordinary temperatures but liquid at the rectal temperature and will therefore melt in the rectum and release the drug. When formulated as a suppository the compositions of the invention may be formulated with traditional binders and carriers, such as triglycerides.

According to another embodiment, the tablet is a compressed suppository or insert. For preparing suppositories, a low melting wax such as a mixture of fatty acid glycerides, such as cocoa butter, is first melted, and the active ingredient is dispersed homogeneously therein by stirring or similar mixing. The molten homogeneous mixture is then poured into convenient sized molds, allowed to cool and thereby solidify.

Parenteral Administration

The compositions of the described invention may be in the form of a sterile injectable aqueous or oleaginous suspension. Injectable preparations, such as sterile injectable aqueous or oleaginous suspensions, may be formulated according to the known art using suitable dispersing or wetting agents and suspending agents.

The sterile injectable preparation may also be a sterile injectable solution or suspension in a nontoxic parenterally acceptable diluent or solvent, for example, as a solution in 1, 3-butanediol. A solution generally is considered as a homogeneous mixture of two or more substances; it is frequently, though not necessarily, a liquid. In a solution, the molecules of the solute (or dissolved substance) are uniformly distributed among those of the solvent. A suspension is a dispersion (mixture) in which a finely-divided species is combined with another species, with the former being so finely divided and mixed that it does not rapidly settle out. In everyday life, the most common suspensions are those of solids in liquid water. Among the acceptable vehicles and solvents that may be employed are water, Ringer's solution, and isotonic sodium chloride solution. In addition, sterile, fixed oils are conventionally employed as a solvent or suspending medium. For parenteral application, particularly suitable vehicles consist of solutions, preferably oily or aqueous solutions, as well as suspensions, emulsions, or implants. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension also may contain suitable stabilizers or agents, which increase the solubility of the compounds to allow for the preparation of highly concentrated solutions. Alternatively, the active compounds may be in powder form for constitution with a suitable vehicle, e.g., sterile pyrogen-free water, before use.

The N-acetyl cysteine, when it is desirable to deliver it locally, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form.

The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Suitable liquid or solid pharmaceutical preparation forms are, for example, microencapsulated, and if appropriate, with one or more excipients, encochleated, coated onto microscopic gold particles, contained in liposomes, pellets for implantation into the tissue, or dried onto an object to be rubbed into the tissue. Such pharmaceutical compositions also may be in the form of granules, beads, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, or solubilizers are customarily used as described above. The pharmaceutical compositions are suitable for use in a variety of drug delivery systems. For a brief review of methods for drug delivery, see Langer 1990 Science 249, 1527-1533, which is incorporated herein by reference.

Injectable depot forms are made by forming microencapsulated matrices of a described inhibitor in biodegradable polymers such as polylactide-polyglycolide. Depending upon the ratio of inhibitor to polymer and the nature of the particular polymer employed, the rate of drug release may be controlled. Such long acting formulations may be formulated with suitable polymeric or hydrophobic materials (for example as an emulsion in an acceptable oil) or ion exchange resins, or as sparingly soluble derivatives, for example, as a sparingly soluble salt. Examples of other biodegradable polymers include poly(orthoesters) and poly(anhydrides). Depot injectable formulations also are prepared by entrapping the inhibitor of the described invention in liposomes or microemulsions, which are compatible with body tissues.

The locally injectable formulations may be sterilized, for example, by filtration through a bacterial-retaining filter or by incorporating sterilizing agents in the form of sterile solid compositions that may be dissolved or dispersed in sterile water or other sterile injectable medium just prior to use. Injectable preparations, for example, sterile injectable aqueous or oleaginous suspensions may be formulated according to the known art using suitable dispersing or wetting agents and suspending agents. The sterile injectable preparation also may be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils conventionally are employed or as a solvent or suspending medium. For this purpose any bland fixed oil may be employed including synthetic mono- or diglycerides. In addition, fatty acids such as oleic acid are used in the preparation of injectables.

Formulations for parenteral administration include aqueous and non-aqueous sterile injection solutions that may contain anti-oxidants, buffers, bacteriostats and solutes, which render the formulation isotonic with the blood of the intended recipient; and aqueous and non-aqueous sterile suspensions, which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example, saline, water-for-injection, immediately prior to use. Extemporaneous injection solutions and suspensions may be prepared from sterile powders, granules and tablets of the kind previously described.

The pharmaceutical agent or a pharmaceutically acceptable ester, salt, solvate or prodrug thereof may be mixed with other active materials that do not impair the desired action, or with materials that supplement the desired action. Solutions or suspensions used for parenteral, intradermal, subcutaneous, intrathecal, or topical application may include, but are not limited to, for example, the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation may be enclosed in ampoules, disposable syringes or multiple dose vials made of glass or plastic. Administered intravenously, particular carriers are physiological saline or phosphate buffered saline (PBS).

Delivery by Inhalation or Insufflation

The compositions of the described invention may be in the form of a dispersible dry powder for delivery by inhalation or insufflation (either through the mouth or through the nose). Dry powder compositions may be prepared by processes known in the art, such as lyophilization and jet milling, as disclosed in International Patent Publication No. WO 91/16038 and as disclosed in U.S. Pat. No. 6,921,527, the disclosures of which are incorporated by reference. The composition of the described invention is placed within a suitable dosage receptacle in an amount sufficient to provide a subject with a unit dosage treatment. The dosage receptacle is one that fits within a suitable inhalation device to allow for the aerosolization of the dry powder composition by dispersion into a gas stream to form an aerosol and then capturing the aerosol so produced in a chamber having a mouthpiece attached for subsequent inhalation by a subject in need of treatment. Such a dosage receptacle includes any container enclosing the composition known in the art such as gelatin or plastic capsules with a removable portion that allows a stream of gas (e.g., air) to be directed into the container to disperse the dry powder composition. Such containers are exemplified by those shown in U.S. Pat. No. 4,227,522; U.S. Pat. No. 4,192,309; and U.S. Pat. No. 4,105,027. Suitable containers also include those used in conjunction with Glaxo's Ventolin® Rotohaler brand powder inhaler or Fison's Spinhaler® brand powder inhaler. Another suitable unit-dose container which provides a superior moisture barrier is formed from an aluminum foil plastic laminate. The pharmaceutical-based powder is filled by weight or by volume into the depression in the formable foil and hermetically sealed with a covering foil-plastic laminate. Such a container for use with a powder inhalation device is described in U.S. Pat. No. 4,778,054 and is used with Glaxo's Diskhaler® (U.S. Pat. Nos. 4,627,432; 4,811,731; and 5,035,237). All of these references are incorporated herein by reference.

Topical Administration

The compositions of the described invention also may be deliverable transdermally. The transdermal compositions may take the form of creams, lotions, aerosols and/or emulsions and can be included in a transdermal patch of the matrix or reservoir type as are conventional in the art for this purpose. The term “topical” refers to administration of an inventive composition at, or immediately beneath, the point of application. The phrase “topically applying” describes application onto one or more surfaces(s) including epithelial surfaces. Although topical administration, in contrast to transdermal administration, generally provides a local rather than a systemic effect, as used herein, unless otherwise stated or implied, the terms topical administration and transdermal administration are used interchangeably. For the purpose of this application, topical applications shall include mouthwashes and gargles.

Topical administration may also involve the use of transdermal administration such as transdermal patches or iontophoresis devices which are prepared according to techniques and procedures well known in the art. The terms “transdermal delivery system”, transdermal patch” or “patch” refer to an adhesive system placed on the skin to deliver a time released dose of a drug(s) by passage from the dosage form through the skin to be available for distribution via the systemic circulation. Transdermal patches are a well-accepted technology used to deliver a wide variety of pharmaceuticals, including, but not limited to, scopolamine for motion sickness, nitroglycerin for treatment of angina pectoris, clonidine for hypertension, estradiol for post-menopausal indications, and nicotine for smoking cessation.

Patches suitable for use in the described invention include, but are not limited to, (1) the matrix patch; (2) the reservoir patch; (3) the multi-laminate drug-inadhesive patch; and (4) the monolithic drug-in-adhesive patch; TRANSDERMAL AND TOPICAL DRUG DELIVERY SYSTEMS, pp. 249-297 (Tapash K. Ghosh et al. eds., 1997), hereby incorporated herein by reference. These patches are well known in the art and generally available commercially.

### Additional Components

The compositions of the described invention may further include conventional excipients, i.e., pharmaceutically acceptable organic or inorganic carrier substances suitable for parenteral application which do not deleteriously react with the active compounds. Suitable pharmaceutically acceptable carriers include, but are not limited to, water, salt solutions, alcohol, vegetable oils, polyethylene glycols, gelatin, lactose, amylose, magnesium stearate, talc, silicic acid, viscous paraffin, perfume oil; fatty acid monoglycerides and diglycerides, petroethral fatty acid esters, hydroxymethylcellulose, polyvinylpyrrolidone, etc.

The compositions may be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds. For parenteral application, suitable vehicles include solutions, such as oily or aqueous solutions, as well as suspensions, emulsions, or implants.

Aqueous suspensions may contain substances which increase the viscosity of the suspension and include, for example, but not limited to, sodium carboxymethyl cellulose, sorbitol and/or dextran. Optionally, the suspension also may contain stabilizers.

These compositions also may contain adjuvants including preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

The composition, if desired, also may contain minor amounts of wetting or emulsifying agents or pH buffering agents. Oral formulations can include standard carriers such as pharmaceutical grades of mannitol, lactose, starch, magnesium stearate, sodium saccharine, cellulose, magnesium carbonate, etc. Examples of suitable buffering agents include, without limitation: acetic acid and a salt (1%-2% w/v); citric acid and a salt (1%-3% w/v); boric acid and a salt (0.5%-2.5% w/v); and phosphoric acid and a salt (0.8%-2% w/v). Suitable preservatives include benzalkonium chloride (0.003%-0.03% w/v); chlorobutanol (0.3%-0.9% w/v); parabens (0.01%-0.25% w/v) and thimerosal (0.004%-0.02% w/v).

### Pharmaceutically Acceptable Carrier

The pharmaceutical compositions within the described invention contain a therapeutically effective amount of N-acetyl cysteine and optionally other therapeutic agents included in a pharmaceutically-acceptable carrier. The components of the pharmaceutical compositions also are capable of being commingled in a manner such that there is no interaction which would substantially impair the desired pharmaceutical efficiency.

The therapeutic agent(s), including N-acetyl cysteine may be provided in particles. The particles may contain the therapeutic agent(s) in a core surrounded by a coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, etc., and any combination thereof. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules that contain N-acetyl cysteine in a solution or in a semi-solid state. The particles may be of virtually any shape.

Both non-biodegradable and biodegradable polymeric materials may be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels as described by Sawhney et al in Macromolecules (1993) 26, 581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. In order to prolong the effect of a drug, it often is desirable to slow the absorption of the drug from subcutaneous, intrathecal, or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Use of a long-term sustained release formulations may be particularly suitable for treatment of chronic conditions. Long-term sustained release formulations are well-known to those of ordinary skill in the art and include some of the release systems described above.

Pharmaceutically Acceptable Salts

Depending upon the structure, the N-acetyl cysteine, and optionally at least one other therapeutic agent, may be administered per se (neat) or, depending upon the structure of the inhibitor, in the form of a pharmaceutically acceptable salt. TN-acetyl cysteine may form pharmaceutically acceptable salts with organic or inorganic acids, or organic or inorganic bases. When used in medicine the salts should be pharmaceutically acceptable, but non-pharmaceutically acceptable salts conveniently may be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts may be prepared as alkaline metal or alkaline earth salts, such as sodium, potassium or calcium salts of the carboxylic acid group.

By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002).

The salts may be prepared in situ during the final isolation and purification of the compounds described within the present invention or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate(isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides, such as methyl, ethyl, propyl, and butyl chlorides, bromides and iodides; dialkyl sulfates like dimethyl, diethyl, dibutyl and diamyl sulfates; long chain halides, such as decyl, lauryl, myristyl and stearyl chlorides, bromides and iodides; arylalkyl halides, such as benzyl and phenethyl bromides, and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid. Basic addition salts may be prepared in situ during the final isolation and purification of compounds described within the invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts may be also obtained using standard procedures well known in the art, for example by reacting with a sufficiently basic compound such as an amine with a suitable acid affording a physiologically acceptable anion. Alkali metal (for example, sodium, potassium or lithium) or alkaline earth metal (for example calcium or magnesium) salts of carboxylic acids may also be made.

### Kits Comprising N-Acetyl Cysteine

According to another aspect, the described invention provides kits for treating a lupus condition in a subject in need thereof, comprising:

(a) a first packaging material containing a composition comprising a therapeutic amount of a compound N-acetyl-L-cysteine (NAC) of Formula I:

[chemical structure]

or a pharmaceutically acceptable salt, solvate, produrg, or a derivative thereof; and a pharmaceutically acceptable carrier; and

(b) a means for administering the composition.

According to one embodiment, the lupus condition is systemic lupus erythematosus (SLE). According to some such embodiments, the systemic lupus erythematosus (SLE) is characterized by at least four of American College of Rheumatology (ACR) criteria selected from the group consisting of a malar rash, a discoid rash, a photosensitivity rash, an oral ulcer, a nonerosive arthritic condition, pleuritis, pericarditis, a renal disorder, a neurologic disorder, a hematologic disorder, an immunologic disorder, or a positive antinuclear antibody test. According to some embodiments, the renal disorder is persistent proteinuria or a cellular cast. According to some embodiments, the neurologic disorder is a seizure or a psychosis. According to some embodiments, the hematologic disorder is hemolytic anemia, leucopenia, lymphopenia, or thrombocytopenia. According to another embodiment, the lupus condition is discoid lupus erythematosus. According to another embodiment, the lupus condition is neonatal lupus erythematosus. According to another embodiment, the lupus condition is drug-induced lupus erythematosus.

According to some embodiments, the therapeutic amount of N-acetyl-L-cysteine (NAC) is from about 1 mg/day to about 8000 mg/day. According to one embodiment, the therapeutic amount of N-acetyl-L-cysteine (NAC) is about 8000 mg/day. According to another embodiment, the therapeutic amount of N-acetyl-L-cysteine (NAC) is about 7800 mg/day. According to another embodiment, the therapeutic amount of N-acetyl-L-cysteine (NAC) is about 7600 mg/day. According to another embodiment, the therapeutic amount of N-acetyl-L-cysteine (NAC) is about 7400 mg/day. According to another embodiment, the therapeutic amount of N-acetyl-L-cysteine (NAC) is about 7200 mg/day. According to another embodiment, the therapeutic amount of N-acetyl-L-cysteine (NAC) is about 7000 mg/day. According to another embodiment, the therapeutic amount of N-acetyl-L-cysteine (NAC) is about 6800 mg/day. According to another embodiment, the therapeutic amount of N-acetyl-L-cysteine (NAC) is about 6600 mg/day. According to another embodiment, the therapeutic amount of N-acetyl-L-cysteine (NAC) is about 6400 mg/day. According to another embodiment, the therapeutic amount of N-acetyl-L-cysteine (NAC) is about 6200 mg/day. According to another embodiment, the therapeutic amount of N-acetyl-L-cysteine (NAC) is about 6000 mg/day. According to another embodiment, the therapeutic amount of N-acetyl-L-cysteine (NAC) is about 5800 mg/day. According to another embodiment, the therapeutic amount of N-acetyl-L-cysteine (NAC) is about 5600 mg/day. According to another embodiment, the therapeutic amount of N-acetyl-L-cysteine (NAC) is about 5400 mg/day. According to another embodiment, the therapeutic amount of N-acetyl-L-cysteine (NAC) is about 5200 mg/day. According to another embodiment, the therapeutic amount of N-acetyl-L-cysteine (NAC) is about 5000 mg/day. According to another embodiment, the therapeutic amount of N-acetyl-L-cysteine (NAC) is about 4800 mg/day. According to another embodiment, the therapeutic amount of N-acetyl-L-cysteine (NAC) is about 4600 mg/day. According to another embodiment, the therapeutic amount of N-acetyl-L-cysteine (NAC) is about 4400 mg/day. According to another embodiment, the therapeutic amount of N-acetyl-L-cysteine (NAC) is about 4200 mg/day. According to another embodiment, the therapeutic amount of N-acetyl-L-cysteine (NAC) is about 4000 mg/day. According to another embodiment, the therapeutic amount of N-acetyl-L-cysteine (NAC) is about 3800 mg/day. According to another embodiment, the therapeutic amount of N-acetyl-L-cysteine (NAC) is about 3600 mg/day. According to another embodiment, the therapeutic amount of N-acetyl-L-cysteine (NAC) is about 3400 mg/day. According to another embodiment, the therapeutic amount of N-acetyl-L-cysteine (NAC) is about 3200 mg/day. According to another embodiment, the therapeutic amount of N-acetyl-L-cysteine (NAC) is about 3000 mg/day. According to another embodiment, the therapeutic amount of N-acetyl-L-cysteine (NAC) is about 2800 mg/day. According to another embodiment, the therapeutic amount of N-acetyl-L-cysteine (NAC) is about 2600 mg/day. According to another embodiment, the therapeutic amount of N-acetyl-L-cysteine (NAC) is about 2400 mg/day. According to another embodiment, the therapeutic amount of N-acetyl-L-cysteine (NAC) is about 2200 mg/day. According to another embodiment, the therapeutic amount of N-acetyl-L-cysteine (NAC) is about 2000 mg/day. According to another embodiment, the therapeutic amount of N-acetyl-L-cysteine (NAC) is about 1800 mg/day. According to another embodiment, the therapeutic amount of N-acetyl-L-cysteine (NAC) is about 1600 mg/day. According to another embodiment, the therapeutic amount of N-acetyl-L-cysteine (NAC) is about 1400 mg/day. According to another embodiment, the therapeutic amount of N-acetyl-L-cysteine (NAC) is about 1200 mg/day. According to another embodiment, the therapeutic amount of N-acetyl-L-cysteine (NAC) is about 1000 mg/day. According to another embodiment, the therapeutic amount of N-acetyl-L-cysteine (NAC) is about 900 mg/day. According to another embodiment, the therapeutic amount of N-acetyl-L-cysteine (NAC) is about 800 mg/day. According to another embodiment, the therapeutic amount of N-acetyl-L-cysteine (NAC) is about 700 mg/day. According to another embodiment, the therapeutic amount of N-acetyl-L-cysteine (NAC) is about 600 mg/day. According to another embodiment, the therapeutic amount of N-acetyl-L-cysteine (NAC) is about 500 mg/day. According to another embodiment, the therapeutic amount of N-acetyl-L-cysteine (NAC) is about 450 mg/day. According to another embodiment, the therapeutic amount of N-acetyl-L-cysteine (NAC) is about 400 mg/day. According to another embodiment, the therapeutic amount of N-acetyl-L-cysteine (NAC) is about 350 mg/day. According to another embodiment, the therapeutic amount of N-acetyl-L-cysteine (NAC) is about 300 mg/day. According to another embodiment, the therapeutic amount of N-acetyl-L-cysteine (NAC) is about 250 mg/day. According to another embodiment, the therapeutic amount of N-acetyl-L-cysteine (NAC) is about 200 mg/day. According to another embodiment, the therapeutic amount of N-acetyl-L-cysteine (NAC) is about 150 mg/day. According to another embodiment, the therapeutic amount of N-acetyl-L-cysteine (NAC) is about 125 mg/day. According to another embodiment, the therapeutic amount of N-acetyl-L-cysteine (NAC) is about 100 mg/day. According to another embodiment, the therapeutic amount of N-acetyl-L-cysteine (NAC) is about 75 mg/day. According to another embodiment, the therapeutic amount of N-acetyl-L-cysteine (NAC) is about 50 mg/day. According to another embodiment, the therapeutic amount of N-acetyl-L-cysteine (NAC) is about 25 mg/day. According to another embodiment, the therapeutic amount of N-acetyl-L-cysteine (NAC) is about 10 mg/day. According to another embodiment, the therapeutic amount of N-acetyl-L-cysteine (NAC) is about 5 mg/day. According to another embodiment, the therapeutic amount of N-acetyl-L-cysteine (NAC) is about 1 mg/day.

According to some embodiments, the maximum daily adult dose of N-acetyl-L-cysteine (NAC) is from about 1 mg/kg body weight to about 100 mg/kg body weight. According to one embodiment, the maximum daily adult dose of N-acetyl-L-cysteine (NAC) is about 1 mg/kg body weight. According to another embodiment, the maximum daily adult dose of N-acetyl-L-cysteine (NAC) is about 2 mg/kg body weight. According to another embodiment, the maximum daily adult dose of N-acetyl-L-cysteine (NAC) is about 4 mg/kg body weight. According to another embodiment, the maximum daily adult dose of N-acetyl-L-cysteine (NAC) is about 6 mg/kg body weight. According to another embodiment, the maximum daily adult dose of N-acetyl-L-cysteine (NAC) is about 8 mg/kg body weight. According to another embodiment, the maximum daily adult dose of N-acetyl-L-cysteine (NAC) is about 10 mg/kg body weight. According to another embodiment, the maximum daily adult dose of N-acetyl-L-cysteine (NAC) is about 12 mg/kg body weight. According to another embodiment, the maximum daily adult dose of N-acetyl-L-cysteine (NAC) is about 14 mg/kg body weight. According to another embodiment, the maximum daily adult dose of N-acetyl-L-cysteine (NAC) is about 16 mg/kg body weight. According to another embodiment, the maximum daily adult dose of N-acetyl-L-cysteine (NAC) is about 18 mg/kg body weight. According to another embodiment, the maximum daily adult dose of N-acetyl-L-cysteine (NAC) is about 20 mg/kg body weight. According to another embodiment, the maximum daily adult dose of N-acetyl-L-cysteine (NAC) is about 22 mg/kg body weight. According to another embodiment, the maximum daily adult dose of N-acetyl-L-cysteine (NAC) is about 24 mg/kg body weight. According to another embodiment, the maximum daily adult dose of N-acetyl-L-cysteine (NAC) is about 26 mg/kg body weight. According to another embodiment, the maximum daily adult dose of N-acetyl-L-cysteine (NAC) is about 28 mg/kg body weight. According to another embodiment, the maximum daily adult dose of N-acetyl-L-cysteine (NAC) is about 30 mg/kg body weight. According to another embodiment, the maximum daily adult dose of N-acetyl-L-cysteine (NAC) is about 32 mg/kg body weight. According to another embodiment, the maximum daily adult dose of N-acetyl-L-cysteine (NAC) is about 34 mg/kg body weight. According to another embodiment, the maximum daily adult dose of N-acetyl-L-cysteine (NAC) is about 36 mg/kg body weight. According to another embodiment, the maximum daily adult dose of N-acetyl-L-cysteine (NAC) is about 38 mg/kg body weight. According to another embodiment, the maximum daily adult dose of N-acetyl-L-cysteine (NAC) is about 40 mg/kg body weight. According to another embodiment, the maximum daily adult dose of N-acetyl-L-cysteine (NAC) is about 42 mg/kg body weight. According to another embodiment, the maximum daily adult dose of N-acetyl-L-cysteine (NAC) is about 44 mg/kg body weight. According to another embodiment, the maximum daily adult dose of N-acetyl-L-cysteine (NAC) is about 46 mg/kg body weight. According to another embodiment, the maximum daily adult dose of N-acetyl-L-cysteine (NAC) is about 48 mg/kg body weight. According to another embodiment, the maximum daily adult dose of N-acetyl-L-cysteine (NAC) is about 50 mg/kg body weight. According to another embodiment, the maximum daily adult dose of N-acetyl-L-cysteine (NAC) is about 52 mg/kg body weight. According to another embodiment, the maximum daily adult dose of N-acetyl-L-cysteine (NAC) is about 54 mg/kg body weight. According to another embodiment, the maximum daily adult dose of N-acetyl-L-cysteine (NAC) is about 56 mg/kg body weight. According to another embodiment, the maximum daily adult dose of N-acetyl-L-cysteine (NAC) is about 58 mg/kg body weight. According to another embodiment, the maximum daily adult dose of N-acetyl-L-cysteine (NAC) is about 60 mg/kg body weight. According to another embodiment, the maximum daily adult dose of N-acetyl-L-cysteine (NAC) is about 62 mg/kg body weight. According to another embodiment, the maximum daily adult dose of N-acetyl-L-cysteine (NAC) is about 64 mg/kg body weight. According to another embodiment, the maximum daily adult dose of N-acetyl-L-cysteine (NAC) is about 66 mg/kg body weight. According to another embodiment, the maximum daily adult dose of N-acetyl-L-cysteine (NAC) is about 68 mg/kg body weight. According to another embodiment, the maximum daily adult dose of N-acetyl-L-cysteine (NAC) is about 70 mg/kg body weight. According to another embodiment, the maximum daily adult dose of N-acetyl-L-cysteine (NAC) is about 72 mg/kg body weight. According to another embodiment, the maximum daily adult dose of N-acetyl-L-cysteine (NAC) is about 74 mg/kg body weight. According to another embodiment, the maximum daily adult dose of N-acetyl-L-cysteine (NAC) is about 76 mg/kg body weight. According to another embodiment, the maximum daily adult dose of N-acetyl-L-cysteine (NAC) is about 78 mg/kg body weight. According to another embodiment, the maximum daily adult dose of N-acetyl-L-cysteine (NAC) is about 80 mg/kg body weight. According to another embodiment, the maximum daily adult dose of N-acetyl-L-cysteine (NAC) is about 82 mg/kg body weight. According to another embodiment, the maximum daily adult dose of N-acetyl-L-cysteine (NAC) is about 84 mg/kg body weight. According to another embodiment, the maximum daily adult dose of N-acetyl-L-cysteine (NAC) is about 86 mg/kg body weight. According to another embodiment, the maximum daily adult dose of N-acetyl-L-cysteine (NAC) is about 88 mg/kg body weight. According to another embodiment, the maximum daily adult dose of N-acetyl-L-cysteine (NAC) is about 90 mg/kg body weight. According to another embodiment, the maximum daily adult dose of N-acetyl-L-cysteine (NAC) is about 92 mg/kg body weight. According to another embodiment, the maximum daily adult dose of N-acetyl-L-cysteine (NAC) is about 94 mg/kg body weight. According to another embodiment, the maximum daily adult dose of N-acetyl-L-cysteine (NAC) is about 96 mg/kg body weight. According to another embodiment, the maximum daily adult dose of N-acetyl-L-cysteine (NAC) is about 98 mg/kg body weight. According to another embodiment, the maximum daily adult dose of N-acetyl-L-cysteine (NAC) is about 100 mg/kg body weight.

According to some embodiments, the maximum daily pediatric dose of N-acetyl-L-cysteine (NAC) is from about 0.1 mg/kg body weight to about 11 mg/kg body weight. According to one embodiment, the maximum daily pediatric dose of N-acetyl-L-cysteine (NAC) is about 0.1 mg/kg body weight. According to another embodiment, the maximum daily pediatric dose of N-acetyl-L-cysteine (NAC) is about 0.2 mg/kg body weight. According to another embodiment, the maximum daily pediatric dose of N-acetyl-L-cysteine (NAC) is about 0.4 mg/kg body weight. According to another embodiment, the maximum daily pediatric dose of N-acetyl-L-cysteine (NAC) is about 0.6 mg/kg body weight. According to another embodiment, the maximum daily pediatric dose of N-acetyl-L-cysteine (NAC) is about 0.8 mg/kg body weight. According to another embodiment, the maximum daily pediatric dose of N-acetyl-L-cysteine (NAC) is about 1.0 mg/kg body weight. According to another embodiment, the maximum daily pediatric dose of N-acetyl-L-cysteine (NAC) is about 1.2 mg/kg body weight. According to another embodiment, the maximum daily pediatric dose of N-acetyl-L-cysteine (NAC) is about 1.4 mg/kg body weight. According to another embodiment, the maximum daily pediatric dose of N-acetyl-L-cysteine (NAC) is about 1.6 mg/kg body weight. According to another embodiment, the maximum daily pediatric dose of N-acetyl-L-cysteine (NAC) is about 1.8 mg/kg body weight. According to another embodiment, the maximum daily pediatric dose of N-acetyl-L-cysteine (NAC) is about 2.0 mg/kg body weight. According to another embodiment, the maximum daily pediatric dose of N-acetyl-L-cysteine (NAC) is about 2.2 mg/kg body weight. According to another embodiment, the maximum daily pediatric dose of N-acetyl-L-cysteine (NAC) is about 2.4 mg/kg body weight. According to another embodiment, the maximum daily pediatric dose of N-acetyl-L-cysteine (NAC) is about 2.6 mg/kg body weight. According to another embodiment, the maximum daily pediatric dose of N-acetyl-L-cysteine (NAC) is about 2.8 mg/kg body weight. According to another embodiment, the maximum daily pediatric dose of N-acetyl-L-cysteine (NAC) is about 3.0 mg/kg body weight. According to another embodiment, the maximum daily pediatric dose of N-acetyl-L-cysteine (NAC) is about 3.2 mg/kg body weight. According to another embodiment, the maximum daily pediatric dose of N-acetyl-L-cysteine (NAC) is about 3.4 mg/kg body weight. According to another embodiment, the maximum daily pediatric dose of N-acetyl-L-cysteine (NAC) is about 3.6 mg/kg body weight. According to another embodiment, the maximum daily pediatric dose of N-acetyl-L-cysteine (NAC) is about 3.8 mg/kg body weight. According to another embodiment, the maximum daily pediatric dose of N-acetyl-L-cysteine (NAC) is about 4.0 mg/kg body weight. According to another embodiment, the maximum daily pediatric dose of N-acetyl-L-cysteine (NAC) is about 4.2 mg/kg body weight. According to another embodiment, the maximum daily pediatric dose of N-acetyl-L-cysteine (NAC) is about 4.4 mg/kg body weight. According to another embodiment, the maximum daily pediatric dose of N-acetyl-L-cysteine (NAC) is about 4.6 mg/kg body weight. According to another embodiment, the maximum daily pediatric dose of N-acetyl-L-cysteine (NAC) is about 4.8 mg/kg body weight. According to another embodiment, the maximum daily pediatric dose of N-acetyl-L-cysteine (NAC) is about 5.0 mg/kg body weight. According to another embodiment, the maximum daily pediatric dose of N-acetyl-L-cysteine (NAC) is about 5.2 mg/kg body weight. According to another embodiment, the maximum daily pediatric dose of N-acetyl-L-cysteine (NAC) is about 5.4 mg/kg body weight. According to another embodiment, the maximum daily pediatric dose of N-acetyl-L-cysteine (NAC) is about 5.6 mg/kg body weight. According to another embodiment, the maximum daily pediatric dose of N-acetyl-L-cysteine (NAC) is about 5.8 mg/kg body weight. According to another embodiment, the maximum daily pediatric dose of N-acetyl-L-cysteine (NAC) is about 6.0 mg/kg body weight. According to another embodiment, the maximum daily pediatric dose of N-acetyl-L-cysteine (NAC) is about 6.2 mg/kg body weight. According to another embodiment, the maximum daily pediatric dose of N-acetyl-L-cysteine (NAC) is about 6.4 mg/kg body weight. According to another embodiment, the maximum daily pediatric dose of N-acetyl-L-cysteine (NAC) is about 6.6 mg/kg body weight. According to another embodiment, the maximum daily pediatric dose of N-acetyl-L-cysteine (NAC) is about 6.8 mg/kg body weight. According to another embodiment, the maximum daily pediatric dose of N-acetyl-L-cysteine (NAC) is about 7.0 mg/kg body weight. According to another embodiment, the maximum daily pediatric dose of N-acetyl-L-cysteine (NAC) is about 7.2 mg/kg body weight. According to another embodiment, the maximum daily pediatric dose of N-acetyl-L-cysteine (NAC) is about 7.4 mg/kg body weight. According to another embodiment, the maximum daily pediatric dose of N-acetyl-L-cysteine (NAC) is about 7.6 mg/kg body weight. According to another embodiment, the maximum daily pediatric dose of N-acetyl-L-cysteine (NAC) is about 7.8 mg/kg body weight. According to another embodiment, the maximum daily pediatric dose of N-acetyl-L-cysteine (NAC) is about 8.0 mg/kg body weight. According to another embodiment, the maximum daily pediatric dose of N-acetyl-L-cysteine (NAC) is about 8.2 mg/kg body weight. According to another embodiment, the maximum daily pediatric dose of N-acetyl-L-cysteine (NAC) is about 8.4 mg/kg body weight. According to another embodiment, the maximum daily pediatric dose of N-acetyl-L-cysteine (NAC) is about 8.6 mg/kg body weight. According to another embodiment, the maximum daily pediatric dose of N-acetyl-L-cysteine (NAC) is about 8.8 mg/kg body weight. According to another embodiment, the maximum daily pediatric dose of N-acetyl-L-cysteine (NAC) is about 9.0 mg/kg body weight. According to another embodiment, the maximum daily pediatric dose of N-acetyl-L-cysteine (NAC) is about 9.2 mg/kg body weight. According to another embodiment, the maximum daily pediatric dose of N-acetyl-L-cysteine (NAC) is about 9.4 mg/kg body weight. According to another embodiment, the maximum daily pediatric dose of N-acetyl-L-cysteine (NAC) is about 9.5 mg/kg body weight. According to another embodiment, the maximum daily pediatric dose of N-acetyl-L-cysteine (NAC) is about 9.6 mg/kg body weight. According to another embodiment, the maximum daily pediatric dose of N-acetyl-L-cysteine (NAC) is about 9.7 mg/kg body weight. According to another embodiment, the maximum daily pediatric dose of N-acetyl-L-cysteine (NAC) is about 9.8 mg/kg body weight. According to another embodiment, the maximum daily pediatric dose of N-acetyl-L-cysteine (NAC) is about 9.9 mg/kg body weight. According to another embodiment, the maximum daily pediatric dose of N-acetyl-L-cysteine (NAC) is about 10 mg/kg body weight. According to another embodiment, the maximum daily pediatric dose of N-acetyl-L-cysteine (NAC) is about 10.1 mg/kg body weight. According to another embodiment, the maximum daily pediatric dose of N-acetyl-L-cysteine (NAC) is about 10.2 mg/kg body weight. According to another embodiment, the maximum daily pediatric dose of N-acetyl-L-cysteine (NAC) is about 10.3 mg/kg body weight. According to another embodiment, the maximum daily pediatric dose of N-acetyl-L-cysteine (NAC) is about 10.4 mg/kg body weight. According to another embodiment, the maximum daily pediatric dose of N-acetyl-L-cysteine (NAC) is about 10.5 mg/kg body weight. According to another embodiment, the maximum daily pediatric dose of N-acetyl-L-cysteine (NAC) is about 10.6 mg/kg body weight. According to another embodiment, the maximum daily pediatric dose of N-acetyl-L-cysteine (NAC) is about 10.7 mg/kg body weight. According to another embodiment, the maximum daily pediatric dose of N-acetyl-L-cysteine (NAC) is about 10.8 mg/kg body weight. According to another embodiment, the maximum daily pediatric dose of N-acetyl-L-cysteine (NAC) is about 10.9 mg/kg body weight. According to another embodiment, the maximum daily pediatric dose of N-acetyl-L-cysteine (NAC) is about 11 mg/kg body weight.

According to some embodiments, a compound of Formula I, or a combination thereof may be provided according to the present invention in any of a variety of useful forms, for example as pharmaceutically acceptable salts, as particular crystal forms, etc. According to some embodiments, a prodrug of one or more compounds of the present invention are provided. Various forms of prodrug are known in the art, for example as discussed in Bundgaard (ed.), Design of Prodrugs, Elsevier (1985); Widder et al. (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975), the entire disclosure of each of which is incorporated herein by reference.

According to one embodiment, the therapeutic amount of the N-acetyl-L-cysteine (NAC) is effective to reduce lupus disease activity in the subject compared to an untreated control. According to some such embodiments, the lupus disease activity is measured by a disease activity score selected from the group consisting of systemic lupus erythematosus disease activity index (SLEDAI) score, British Isles Lupus Assessment Group (BILAG) score, fatigue assessment scale (FAS) score, or a combination thereof.

According to some such embodiments, the therapeutic amount of N-acetyl-L-cysteine (NAC) is effective to reduce systemic lupus erythematosus disease activity index (SLEDAI) compared to an untreated control at least after at least 7 days of the administration, after at least 14 days of the administration, after at least 1 month of the administration, after at least 2 months of the administration, after at least 3 months of the administration, or after at least 4 months of the administration. According to some such embodiments, the therapeutic amount of N-acetyl-L-cysteine (NAC) is effective to reduce systemic lupus erythematosus disease activity index (SLEDAI) by at least 1 point compared to an untreated control after at least 1 month of the administration. According to some such embodiments, the therapeutic amount of N-acetyl-L-cysteine (NAC) is effective to reduce systemic lupus erythematosus disease activity index (SLEDAI) by at least 1.1 points compared to an untreated control after at least 1 month of the administration. According to some such embodiments, the therapeutic amount of N-acetyl-L-cysteine (NAC) is effective to reduce systemic lupus erythematosus disease activity index (SLEDAI) by at least 1.5 points compared to an untreated control after at least 1 month of the administration. According to some such embodiments, the therapeutic amount of N-acetyl-L-cysteine (NAC) is effective to reduce systemic lupus erythematosus disease activity index (SLEDAI) by at least 2.0 points compared to an untreated control after at least 1 month of the administration. According to some such embodiments, the therapeutic amount of N-acetyl-L-cysteine (NAC) is effective to reduce systemic lupus erythematosus disease activity index (SLEDAI) by at least 2.1 points compared to an untreated control after at least 1 month of the administration. According to some such embodiments, the therapeutic amount of N-acetyl-L-cysteine (NAC) is effective to reduce systemic lupus erythematosus disease activity index (SLEDAI) by at least 2.2 points compared to an untreated control after at least 1 month of the administration. According to some such embodiments, the therapeutic amount of N-acetyl-L-cysteine (NAC) is effective to reduce systemic lupus erythematosus disease activity index (SLEDAI) by at least 2.3 points compared to an untreated control after at least 1 month of the administration.

According to some such embodiments, the therapeutic amount of N-acetyl-L-cysteine (NAC) is effective to reduce British Isles Lupus Assessment Group (BILAG) score compared to an untreated control after at least 7 days of the administration, at least after 14 days of the administration, at least after 1 month of the administration, at least after 2 months of the administration, at least after 3 months of the administration, or at least after 4 months of the administration. According to some such embodiments, the therapeutic amount of N-acetyl-L-cysteine (NAC) is effective to reduce British Isles Lupus Assessment Group (BILAG) score by at least 1 point compared to an untreated control after at least 1 month of the administration. According to some such embodiments, the therapeutic amount of N-acetyl-L-cysteine (NAC) is effective to reduce British Isles Lupus Assessment Group (BILAG) score by at least 2.0 points compared to an untreated control after at least 1 month of the administration. According to some such embodiments, the therapeutic amount of N-acetyl-L-cysteine (NAC) is effective to reduce British Isles Lupus Assessment Group (BILAG) score by at least 2.5 points compared to an untreated control after at least 1 month of the administration. According to some such embodiments, the therapeutic amount of N-acetyl-L-cysteine (NAC) is effective to reduce British Isles Lupus Assessment Group (BILAG) score by at least 3.0 points compared to an untreated control after at least 1 month of the administration. According to some such embodiments, the therapeutic amount of N-acetyl-L-cysteine (NAC) is effective to reduce British Isles Lupus Assessment Group (BILAG) score by at least 3.5 points compared to an untreated control after at least 1 month of the administration. According to some such embodiments, the therapeutic amount of N-acetyl-L-cysteine (NAC) is effective to reduce British Isles Lupus Assessment Group (BILAG) score by at least 4.0 points compared to an untreated control after at least 1 month of the administration. According to some such embodiments, the therapeutic amount of N-acetyl-L-cysteine (NAC) is effective to reduce British Isles Lupus Assessment Group (BILAG) score by at least 4.5 points compared to an untreated control after at least 1 month of the administration. According to some such embodiments, the therapeutic amount of N-acetyl-L-cysteine (NAC) is effective to reduce British Isles Lupus Assessment Group (BILAG) score by at least 5.0 points compared to an untreated control after at least 1 month of the administration.

According to some such embodiments, the therapeutic amount of N-acetyl-L-cysteine (NAC) is effective to reduce fatigue assessment scale (FAS) score compared to an untreated control after at least 7 days of the administration, at least after 14 days of the administration, at least after 1 month of the administration, at least after 2 months of the administration, at least after 3 months of the administration, or at least after 4 months of the administration. According to some such embodiments, the therapeutic amount of N-acetyl-L-cysteine (NAC) is effective to reduce fatigue assessment scale (FAS) score by at least 1 point compared to an untreated control after at least 1 month of the administration. According to some such embodiments, the therapeutic amount of N-acetyl-L-cysteine (NAC) is effective to reduce fatigue assessment scale (FAS) score by at least 2.0 points compared to an untreated control after at least 1 month of the administration. According to some such embodiments, the therapeutic amount of N-acetyl-L-cysteine (NAC) is effective to reduce fatigue assessment scale (FAS) score by at least 2.5 points compared to an untreated control after at least 1 month of the administration. According to some such embodiments, the therapeutic amount of N-acetyl-L-cysteine (NAC) is effective to reduce fatigue assessment scale (FAS) score by at least 3.0 points compared to an untreated control after at least 1 month of the administration. According to some such embodiments, the therapeutic amount of N-acetyl-L-cysteine (NAC) is effective to reduce fatigue assessment scale (FAS) score by at least 3.5 points compared to an untreated control after at least 1 month of the administration. According to some such embodiments, the therapeutic amount of N-acetyl-L-cysteine (NAC) is effective to reduce fatigue assessment scale (FAS) score by at least 4.0 points compared to an untreated control after at least 1 month of the administration. According to some such embodiments, the therapeutic amount of N-acetyl-L-cysteine (NAC) is effective to reduce fatigue assessment scale (FAS) score by at least 4.5 points compared to an untreated control after at least 1 month of the administration. According to some such embodiments, the therapeutic amount of N-acetyl-L-cysteine (NAC) is effective to reduce fatigue assessment scale (FAS) score by at least 5.0 points compared to an untreated control after at least 1 month of the administration.

According to one embodiment, the therapeutic amount of N-acetyl-L-cysteine (NAC) is effective to increase mitochondrial mass of T cells of the subject compared to an untreated control. According to some embodiments, the therapeutic amount of N-acetyl-L-cysteine (NAC) is effective to increase mitochondrial mass of T cells of the subject compared to compared to an untreated control after at least 7 days of the administration, after at least 14 days of the administration, after at least 1 month of the administration, after at least 2 months of the administration, after at least 3 months of the administration, or after at least 4 months of the administration.

According to another embodiment, the therapeutic amount of N-acetyl-L-cysteine (NAC) is effective to increase mitochondrial membrane potential in double negative (DN) T cells of the subject compared to an untreated control. According to some embodiments, the therapeutic amount of N-acetyl-L-cysteine (NAC) is effective to increase mitochondrial membrane potential in double negative (DN) T cells of the subject compared to an untreated control after at least 7 days of the administration, after at least 14 days of the administration, after at least 1 month of the administration, after at least 2 months of the administration, after at least 3 months of the administration, or after at least 4 months of the administration.

According to another embodiment, the therapeutic amount of N-acetyl-L-cysteine (NAC) is effective to increase level of a reactive oxygen intermediate (ROI) in double negative (DN) T cells of the subject compared to an untreated control. According to one embodiment, the oxygen intermediate (ROI) is hydrogen peroxide. According to some embodiments, the therapeutic amount of N-acetyl-L-cysteine (NAC) is effective to increase level of a reactive oxygen intermediate (ROI) in double negative (DN) T cells of the subject compared to an untreated control after at least 7 days of the administration, after at least 14 days of the administration, after at least 1 month of the administration, after at least 2 months of the administration, after at least 3 months of the administration, or after at least 4 months of the administration.

According to another embodiment, the therapeutic amount of N-acetyl-L-cysteine (NAC) is effective to increase spontaneous apoptotic rate of double negative (DN) T cells of the subject compared to an untreated control. According to some embodiments, the therapeutic amount of N-acetyl-L-cysteine (NAC) is effective to increase spontaneous apoptotic rate of double negative (DN) T cells of the subject compared to an untreated control after at least 7 days of the administration, after at least 14 days of the administration, after at least 1 month of the administration, after at least 2 months of the administration, after at least 3 months of the administration, or after at least 4 months of the administration.

According to another embodiment, the therapeutic amount of N-acetyl-L-cysteine (NAC) is effective to increase activation-induced apoptotic rate of double negative (DN) T cells of the subject compared to an untreated control. According to some embodiments, the therapeutic amount of N-acetyl-L-cysteine (NAC) is effective to increase activation-induced apoptotic rate of double negative (DN) T cells of the subject compared to an untreated control after at least 7 days of the administration, after at least 14 days of the administration, after at least 1 month of the administration, after at least 2 months of the administration, after at least 3 months of the administration, or after at least 4 months of the administration.

According to another embodiment, the therapeutic amount of N-acetyl-L-cysteine (NAC) is effective to decrease activity of mammalian target of rapamycin (mTOR) compared to an untreated control. According to some embodiments, the therapeutic amount of N-acetyl-L-cysteine (NAC) is effective to decrease activity of mammalian target of rapamycin (mTOR) compared to an untreated control after at least 7 days of the administration, after at least 14 days of the administration, after at least 1 month of the administration, after at least 2 months of the administration, after at least 3 months of the administration, or after at least 4 months of the administration. According to some such embodiments, the therapeutic amount of N-acetyl-L-cysteine (NAC) is effective to decrease phosphorylated ribosomal protein S6 (p-RPS6^{high}) cells in double negative (DN) T cells of the subject by at least 2-fold compared to an untreated control.

According to another embodiment, the therapeutic amount of N-acetyl-L-cysteine (NAC) is effective to increase number of FoxP3+CD8+CD25+ T cells compared to an untreated control. According to some embodiments, the therapeutic amount of N-acetyl-L-cysteine (NAC) is effective to increase number of FoxP3+CD8+CD25+ T cells compared to an untreated control after at least 7 days of the administration, after at least 14 days of the administration, after at least 1 month of the administration, after at least 2 months of the administration, after at least 3 months of the administration, or after at least 4 months of the administration.

According to another embodiment, the therapeutic amount of N-acetyl-L-cysteine (NAC) is effective to reduce a cognitive/inattentive component of attention deficit and hyperactivity (ADHD) self-report scale (ASRS) compared to an untreated control. According to another embodiment, the therapeutic amount of N-acetyl-L-cysteine (NAC) is effective to reduce a cognitive/inattentive component of attention deficit and hyperactivity (ADHD) self-report scale (ASRS) compared to an untreated control after at least 7 days of the administration, after at least 14 days of the administration, after at least 1 month of the administration, after at least 2 months of the administration, after at least 3 months of the administration, or after at least 4 months of the administration.

According to some embodiments, the kit further comprises a second packaging material containing at least one additional therapeutic agent. According to another embodiment, the additional therapeutic agent is of a therapeutic amount effective to exert an additive effect in treating or alleviating one or more symptoms of the lupus condition. According to another embodiment, the additional therapeutic agent is of a therapeutic amount effective to exert a synergistic effect in treating or alleviating one or more symptoms of the lupus condition.

According to some embodiments, the additional therapeutic agent is selected from a group consisting of a non-steroidal anti-inflammatory agent, an antimalarial agent, a corticosteroid, a cytotoxic agent, an immunosuppressive agent, a biologic, or a combination thereof. According to one embodiment, the additional therapeutic agent is a non-steroidal anti-inflammatory agent. Exemplary non-steroidal anti-inflammatory agents include but are not limited to salicylate derivates (e.g. aspirin, arthopan), celecoxib (Celebrix®), diclofenac (Cataflam®, Voltaren®), etodolac (Lodine®), fenprofen (Nalfon®), flurbiprofen (Ansaid®), ibuprofen (Motrin®, Advil®, Nuprin®), ketoprofen (Orudis®, Actron®), meclofamate (Meclomen®), meloxicam (Mobic®), nabumetone (Relafen®), naproxen (Aleve®, Naprosyn®, Anaprox®), oxaprozin (Daypro®), piroxicam (Feldene®), rofecoxib (Vioxx®), sulindac (Clinoril®), tolmetin (Tolectin®) and acetaminophen (Tylenol®). According to another embodiment, the additional therapeutic agent is an antimalarial agent. Exemplary antimalarial agents include but are not limited to hydroxycloroquine (Plauenil®), chloroquine (Aralen®), quinicrine (Atabrine®). According to another embodiment, the additional therapeutic agent is a corticosteroid. Exemplary corticocorticosteroids include but are not limited to topical creams or ointments such as clobetasol (Temovate®), halobetasol (Ultravate®), hydrocortisone (Cortel®, Cortaid®), triamcinolone (Aristocort®, Kenalog®), betamethasone (Valisone®, Diprosone®), fluocinolone (Synalar®), fluocinonide (Lidex®); tablets such as prednisone (Deltasone®), prednisolone (Prelone®), ethylprednisone (Medrol®); and intravenous formulations such as methylprednisone (Solu-Medrol®), hydrocortisone (Solu-Cortel®). According to another embodiment, the additional therapeutic agent is a cytotoxic agent. Exemplary cytotoxic agents include but are not limited to azathioprine (Imuran®), cyclophosphamide (Cytoxan®), mycophenolate mofetil (Cellcept®), cyclosporine A (Sandimmune®, Neoral®), methotrexate (Rhematrex®), chlorambucil (Leukeran®). According to another embodiment, the additional therapeutic agent is an immunosuppressive agent. Exemplary immunosuppressive agents include but are not limited to azathioprine (Imuran®), cyclophosphamide (Cytoxan®), mycophenolate mofetil (Cellcept®), cyclosporine A (Sandimmune®, Neoral®), methotrexate (Rhematrex®), chlorambucil (Leukeran®). According to another embodiment, the additional therapeutic agent is a biologic. Exemplary biologics include but are not limited to a B-cell target biologic (Ezpratuzumab®, Rituximab®, Belimumab®), a T cell target biologic (Abatcept, rapamycin), a spleen tyrosine kinase antagonist (R788), a tumor necrosis factor (TNF) antagonist, an interferon antagonist, an interleukin-6-receptor antagonist.

According to some embodiments, the means for administering the composition is a syringe, a nebulizer, an inhaler, a dropper, a syringe, a nebulizer, an inhaler, a dropper, a tablet, a pill, a gel, a troche, a lozenge, an aqueous suspension, an oily suspension, a capsule, a syrup, an emulsion, a cream, a patch, an injectable solution, a granule, a bead, an implant, a suppository, an insert, or a combination thereof

According to some embodiments, the kit further comprises instructions for use. According to some embodiments, the kit further comprises packaging materials. According to some embodiments, the first or second packaging material is selected from the group consisting of a box, a pouch, a vial, a bottle, a tube, a blister pack, or a combination thereof.

According to some embodiments, the composition is in the form of a tablet, a pill, a gel, an injectable solution, an aerosol, a troche, a lozenge, an aqueous suspension, an oily suspension, a dispersible powder, a granule, a bead, an emulsion, an implant, a cream, a patch, a capsule, a syrup, a suppository or an insert. According to one embodiment, the composition is in the form of a tablet. According to another embodiment, the composition is in the form of a pill. According to another embodiment, the composition is in the form of a gel. According to another embodiment, the composition is in the form of an injectable solution. According to another embodiment, the composition is in the form of an aerosol. According to another embodiment, the composition is in the form of a troche. According to another embodiment, the composition is in the form of a lozenge. According to another embodiment, the composition is in the form of an aqueous suspension. According to another embodiment, the composition is in the form an oily suspension. According to another embodiment, the composition is in the form of a dispersible powder. According to another embodiment, the composition is in the form of a granule. According to another embodiment, the composition is in the form of a bead. According to another embodiment, the composition is in the form of an emulsion. According to another embodiment, the composition is in the form of an implant. According to another embodiment, the composition is in the form of a cream. According to another embodiment, the composition is in the form of a patch. According to another embodiment, the composition is in the form of a capsule. According to another embodiment, the composition is in the form of a syrup. According to another embodiment, the composition is in the form of a suppository. According to another embodiment, the composition is in the form of an insert.

### Equivalents

Unless defined otherwise, all technical and scientific terms used herein have the same meaning as commonly understood by one of ordinary skill in the art to which this invention belongs. Although any methods and materials similar or equivalent to those described herein also can be used in the practice or testing of the present invention, the preferred methods and materials are now described. All publications mentioned herein are incorporated herein by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto. Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

It must be noted that as used herein and in the appended claims, the singular forms “a”, “and”, and “the” include plural references unless the context clearly dictates otherwise. All technical and scientific terms used herein have the same meaning

The publications discussed herein are provided solely for their disclosure prior to the filing date of the present application and are incorporated herein by reference in their entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### NAC Treatment of SLE—Double-Blind Placebo-Controlled Pilot Study

### Clinical Study Design

36 patients with stable disease were enrolled in a double-blind placebo-controlled treatment trial with N-acetylcysteine (NAC; FDA approval, IND No: 101,320; clinicaltrials.gov identifier: NCT00775476). SLE patients were randomized to receive either placebo (placebo group—dextrose) or NAC (test group) in one of three treatment arms of increasing doses for three months: 600 mg twice daily (Dose 1), 1,200 mg twice daily (Dose 2), or 2,400 mg twice daily (Dose 3). 12 patients were enrolled per treatment arm, 9 received NAC while 3 received placebo. 6 of 8 active patients needed to tolerate each dose and showed no worsening of SLE as defined in the Data Safety and Monitoring Plan (DSMP) to proceed to the next higher dose.

Table 6 shows the demographic data of SLE patients enrolled into the placebo and three NAC treatment arms: 600 mg twice daily (Dose 1), 1,200 mg twice daily (Dose 2), or 2,400 mg twice daily (Dose 3).

TABLE 6
Demographic data of SLE patients enrolled into the placebo and
three NAC treatment arms: 600 mg twice daily (Dose 1), 1,200 mg
twice daily (Dose 2), or 2,400 mg twice daily (Dose 3).

| | | | | | | | | Age | |
|---|---|---|---|---|---|---|---|---|---|
| | | | | | | | | of | Disease |
| | | | Duration | Side | | Age | | Onset | Duration |
| GROUP | Patient # | Dose | (Days) | Effect | Gender | (yrs) | Ethnicity | (yrs) | (yrs) |
| PLACEBO | | | | | | | | | |
| | AIM1- | 0 | 90 | 0 | Female | 48 | White | 41 | 8 |
| | 003 | | | | | | | | |
| | AIM1- | 0 | 90 | 0 | Female | 35 | White | 25 | 10 |
| | 005 | | | | | | | | |
| | AIM1- | 0 | 90 | 0 | Female | 31 | White | 23 | 9 |
| | 009 | | | | | | | | |
| | AIM1- | 0 | 90 | 0 | Female | 61 | White | 52 | 9 |
| | 015 | | | | | | | | |
| | AIM1- | 0 | <30 | 0 | Female | 26 | White | 21 | 5 |
| | 017 | | | | | | | | |
| | AIM1- | 0 | 90 | 0 | Female | 53 | White | 37 | 17 |
| | 021 | | | | | | | | |
| | AIM1- | 0 | 90 | 0 | Female | 54 | White | 48 | 6 |
| | 027 | | | | | | | | |
| | AIM1- | 0 | 90 | 0 | Male | 51 | White | 49 | 3 |
| | 029 | | | | | | | | |
| | AIM1- | 0 | 90 | 0 | Female | 38 | White | 32 | 11 |
| | 033 | | | | | | | | |
| | Mean | | | | | 44.1 | | 36.44 | 8.67 |
| N-ACETYL | | | | | | | | | |
| CYSTEINE | | | | | | | | | |
| (NAC) | | | | | | | | | |
| | AIM1- | 1 | 90 | 0 | Female | 49 | White | 43 | 6 |
| | 001 | | | | | | | | |
| | AIM1- | 1 | 90 | 0 | Female | 33 | White | 21 | 12 |
| | 002 | | | | | | | | |
| | AIM1- | 1 | 90 | 0 | Female | 56 | White | 53 | 3 |
| | 004 | | | | | | | | |
| | AIM1- | 1 | 90 | 0 | Female | 42 | African | 33 | 9 |
| | 006 | | | | | | American | | |
| | AIM1- | 1 | 90 | 0 | Female | 33 | White | 22 | 11 |
| | 007 | | | | | | | | |
| | AIM1- | 1 | 90 | 0 | Female | 40 | White | 35 | 5 |
| | 008 | | | | | | | | |
| | AIM1- | 1 | 90 | 0 | Female | 54 | White | 46 | 8 |
| | 010 | | | | | | | | |
| | AIM1- | 1 | 90 | 0 | Female | 59 | White | 56 | 2 |
| | 011 | | | | | | | | |
| | AIM1- | 1 | 90 | 0 | Female | 44 | White | 39 | 6 |
| | 012 | | | | | | | | |
| | AIM1- | 2 | 90 | 0 | Female | 25 | White | 24 | 2 |
| | 013 | | | | | | | | |
| | AIM1- | 2 | 90 | 0 | Female | 41 | White- | 27 | 14 |
| | 014 | | | | | | Hispanic | | |
| | AIM1- | 2 | 60 | 0 | Female | 50 | White | 48 | 2 |
| | 016 | | | | | | | | |
| | AIM1- | 2 | 90 | 0 | Female | 46 | African | 30 | 5 |
| | 018 | | | | | | American- | | |
| | | | | | | | Hispanic | | |
| | AIM1- | 2 | 90 | 0 | Female | 64 | White | 60 | 3 |
| | 019 | | | | | | | | |
| | AIM1- | 2 | 90 | 0 | Female | 37 | White | 32 | 4 |
| | 020 | | | | | | | | |
| | AIM1- | 2 | 90 | 0 | Female | 52 | White | 48 | 4 |
| | 022 | | | | | | | | |
| | AIM1- | 2 | 90 | 0 | Female | 48 | African | 43 | 5 |
| | 23 | | | | | | American | | |
| | AIM1- | 2 | 90 | 0 | Female | 31 | White | 30 | 1 |
| | 024 | | | | | | | | |
| | AIM1- | 3 | 90 | 0 | Female | 33 | White | 31 | 1 |
| | 025 | | | | | | | | |
| | AIM1- | 3 | 19 | Heart | Female | 49 | White | 29 | 19 |
| | 026 | | | burn | | | | | |
| | AIM1- | 3 | 60 | Nausea | Male | 25 | White | 23 | 2 |
| | 028 | | | | | | | | |
| | AIM1- | 3 | 90 | 0 | Female | 43 | White | 24 | 14 |
| | 030 | | | | | | | | |
| | AIM1- | 3 | 90 | 0 | Female | 50 | White | 38 | 12 |
| | 031 | | | | | | | | |
| | AIM1- | 3 | 42 | Nausea | Female | 56 | White | 54 | 3 |
| | 032 | | | | | | | | |
| | AIM1- | 3 | 90 | 0 | Female | 60 | White | 56 | 2 |
| | 034 | | | | | | | | |
| | AIM1- | 3 | 90 | 0 | Female | 44 | White | 40 | 3 |
| | 035 | | | | | | | | |
| | AIM1- | 3 | 90 | 0 | Female | 45 | White | 41 | 5 |
| | 036 | | | | | | | | |
| | Mean | | | | | 44.8 | | 38 | 6.04 |

The mean (±SEM) age of patients was 44.6 (±1.8) years, ranging between 25-64 years (Table 6). 34 patients were females including 30 Caucasians, two African-Americans, and two Hispanic. 2 patients were Caucasian males. 42 healthy subjects were individually matched for each patient blood donation for age within ten years, gender, and ethnic background and freshly isolated cells were studied in parallel as controls for immunological studies. The mean (±SEM) age of controls was 44.4 (±1.7) years, ranging between 22-63 years. 39 controls were females including 36 Caucasians, two African-Americans, and one Hispanic. 3 controls were Caucasian males.

Inclusion criteria: patients who fulfilled the following criteria were included in the study:
a. age >18 yr, male or female;
b. SLE with ≧4 of eleven diagnostic criteria approved by the American College of Rheumatology (ACR) (Bombardier, C. et al., “Derivation of the SLEDAI. A disease activity index for lupus patients,” Arth. Rheum., 35: 630-640 (1992); and
c. clinically stable disease on prednisone (≦10 mg/day), anti-malarials, azathioprine or mycophenylate mofetil as allowable immunosuppressant medications.

Exclusion criteria: patients who showed any of the following criteria were excluded from the study:
a. pregnant or lactating;
b. moderately serious or serious co-morbidities (e.g., diabetes mellitus, congestive heart failure, chronic obstructive pulmonary disease, chronic renal insufficiency);
c. history of chronic infections (e.g., HIV, hepatitis B virus, hepatitis C virus, mycobacteria, bronchiectasis);
d. infections in the past month;
e. history of severe or recurrent infections;
f. smokers;
g. Patients taking over-the-counter antioxidants that can enhance the effect of NAC were excluded. Alternatively, patients taking acetaminophen (Tylenol) which is metabolized by hepatic cytochrome P450 enzymes, primarily CYP2E1, to a toxic intermediate compound (N-acetyl-para benzoquine imide), requiring detoxification by hepatic GSH (Benson, G. D. et al., “The therapeutic use of acetaminophen in patients with liver disease,” Am. J. Therapeut., 12: 133-141 (2005)) were excluded;
h. Patients with acute flare of SLE threatening vital organs and requiring intravenous cyclophosphamide treatment;
i. Patients receiving biologicals (rituximab, abatacept); and
j. enrolled in other clinical trials.

One daily dose of multivitamin, containing ≧500 mg of vitamin C and ≦30 IU of vitamin E was allowed for each patient.

### Study Materials

Identical appearing capsules containing NAC or placebo (dextrose) were manufactured by the compounding pharmacy within the Department of Pharmacy at SUNY Upstate Medical Center. Both NAC and dextrose were obtained from Spectrum Chemical Manufacturing Corporation (New Brunswick, N.J.).

Each capsule contained 600 mg NAC or placebo. All capsules were rolled in NAC to equalize odor. Each bottle contained capsules needed for 32 days. The pills were counted when the bottles were returned to ascertain compliance. A study biostatistician worked closely with the Department of Pharmacy to ensure blindness of researchers to participating patients' randomized conditions.

### Patient Visit Schedules

Each patient went through the following visit schedules:

A. Screening visit: patients were evaluated for inclusion and exclusion criteria.

B. Visit No. 1: Baseline assessment of clinical disease activity was performed. Blood was drawn for routine laboratory parameters and immunological parameters and GSH were performed before the first NAC dose. Additional blood samples were drawn 3 h and 6 h after the initial NAC dose for measurement of GSH by HPLC. First monthly supply of NAC or placebo was provided.

C. Visit No. 2: One-month visit: clinical assessment was performed and blood was drawn for routine laboratory tests and measurement of immunological parameters and GSH levels before morning NAC dose. Second monthly supply of NAC or placebo was provided.

D. Visit No. 3: Two-month visit: clinical assessment was performed and blood was drawn for routine laboratory tests and measurement of immunological parameters and GSH levels before morning NAC dose. Third monthly supply of NAC or placebo was provided

E. Visit No. 4: Three-month visit: clinical assessment was performed and blood was drawn for routine laboratory tests and measurement of immunological parameters and GSH levels before morning NAC dose.

F. Visit No. 5: Four-month visit (end of 1 month washout): clinical assessment was performed and blood was drawn for routine laboratory tests and measurement of immunological parameters and GSH levels.

For each patient visit, blood from healthy donors was obtained and matched for age (within one decade), gender, and ethnicity, to be used as control for flow cytometry measurement of mitochondrial function, T-cell activation and death pathway selection, Ca²⁺ flux, production of nitric oxide (NO) and reactive oxygen intermediates (ROI), activation of mTOR and expression of Foxp3 in subsets of T cells and B cells.

GSH was measured in whole blood and isolated peripheral blood lymphocytes (PBL) by HPLC. Each patient provided seven blood samples (visit 1/0h, visit ⅓ h, visit ⅙ h, visit 2 in 1 month, visit 3 in 2 months, visit 4 in 3 months, visit 5 in 4 months (after one month washout). 42 healthy controls also donated blood to use as control for HPLC analysis of GSH, flow cytometry of live cells as well as for the gene expression and signaling studies. ˜384 flow cytometry data points for each of the five patient visits were recorded, both for the patients and the matching controls. DNA, RNA, and protein lysates were saved and catalogued for each visit. Individual controls gave blood on multiple occasions.

### Clinical Outcomes and Assessments

### 1. Tolerance:

At each visit, patients were specifically asked about common side effects (nausea, bloating, bad taste) seen in prior trials These side effects were reviewed by the Data Safety and Monitoring Board (DSMB) bi-annually. Tolerance and safety were primary clinical outcomes.

All 24 patients in Dosing Group 1 (1.2 g/day NAC) and 2 (2.4 g/day NAC) completed the treatment and none of the 12 patients in Dosing Group 1 (1.2 g/day NAC) or 2 (2.4 g/day NAC) reported unpleasant smell or taste. Three of 12 patients in Dosing Group 3 (4.8 g/day) dropped out (Table 6) due to 1) heartburn after 26 days, which resolved after discontinuing the capsules; 2) recurrent nausea and one-time vomiting after 46 days, with resolution after discontinuing the capsules; 3) headaches, which were eliminated by halving the dose to 2.4 g/day. Since all three patients reporting intolerance received NAC, in accordance with the DSMP, no higher dose was initiated. Following the last Dosing Group 3 visit, the study was un-blinded.

This double-blind placebo-controlled phase I/pilot study provides evidence that NAC is safe and tolerated by all SLE patients up to 2.4 g/day with reversible nausea in 33% of patients receiving 4.8 g/day.

### 2. Blinding:

Odor or taste was specifically noted.

### 3. Clinical Assessments:

A complete physical examination of the cardiovascular, respiratory, gastrointestinal, musculoskeletal, neurological systems, skin, head, neck, sinuses, nasal and oral cavities were performed at each visit. SLE disease activity was assessed by using the British Isles Lupus Assessment Group (BILAG) (Isenberg, D. A. et al., “BILAG 2004. Development and initial validation of an updated version of the British Isles Lupus Assessment Group's disease activity index for patients with systemic lupus erythematosus,” Rheumatology 44(7): 902-906 (2005)) and SLE Disease Activity Index (SLEDAI) (Bombardier, C. et al., “Derivation of the SLEDAI. A disease activity index for lupus patients,” Arth. Rheum., 35: 630-640 (1992)). The concurrent use and dosage of prednisone and other medications were documented.

Fatigue was assessed by using a validated Fatigue Assessment Scale (FAS), a self-questionnaire that provides a subjective measurement of fatigue severity and has shown to have a high degree of internal consistency, validity, and sensitivity to changes in clinical condition, as described in Michielsen, H. J. et al., “Psychometric qualities of a brief self-rated fatigue measure: The Fatigue Assessment Scale,” J. Psychosom. Res., 54(4): 345-352 (2003), the entire content of which is incorporated by reference herein.

Lupus disease activity was measured by SLEDAI and BILAG and fatigue was evaluated by FAC at baseline (visit 1) as well as monthly during a 3-month intervention (visits 2-4) and after a 1-month washout period (visit 5). FIG. 1 shows the effect of NAC and placebo on disease activity, as measured by SLEDAI (FIG. 1A), BILAG (FIG. 1B), and FAS scores (FIG. 1C), in 36 SLE patients exposed to placebo (n=9), 1.2 g/day NAC (NAC Dose 1, n=9), 2.4 g/day NAC (NAC Dose 2, n=9), 4.8 g/day NAC (NAC Dose 3, n=9), or all doses of NAC considered together (n=27).

Placebo or NAC dose 1 (1.2 g/day NAC) did not influence SLE disease activity (as measured by SLEDAI, BILAG, or FAS scales) (FIG. 1). NAC dose 2 (2.4 g/day) and dose 3 (4.8 g/day) reduced SLEDAI after 1 month (p=0.0007), 2 months (p=0.0009), 3 months (p=0.0030) and 4 months (p=0.0046) (FIG. 1A). A significant improvement was observed in 2 of 9 patients in the placebo group, 3 of 9 patients in Dose 1 group, 4 of 9 patients in Dose 2 group and 5 of 6 patients in Dose 3 groups. A significant improvement was observed especially with patients who achieved improvements of SLEDAI scores of 3 or more in NAC dosing group 3. (p=0.0406 relative to placebo, using Fischer=exact test). In all patients treated with NAC, SLEDAI was improved from 5.3 at baseline (visit 1) to 3.5 after 1-month (visit 2; p=0.0013) and to 3.7 after 2-month treatment (visit 3; p=0.048; FIG. 1A). In patients treated with NAC doses 2 and 3 combined, SLEDAI improved from 5.78 at baseline on all follow-up visits (visit 2: 3.6, p=0.0007; visit 3: 4.0, p=0.0009; visit 4: 4.9, p=0.0030; visit 5: 4.4, p=0.0046). Using multilevel modeling, the reduction in SLEDAI was greater in the NAC than placebo group as indicated by a significant visit by drug interaction (XTMIXED z=−2.14, p=0.033).

NAC dose 2 (2.4 g/day) and dose 3 (4.8 g/day) reduced BILAG after 1 month (p=0.029) and 3 months (p=0.0009); and 3) FAS after 2 months (p=0.002) and 3 months (p=0.004). In all patients treated with NAC, BILAG improved from 26.2 at baseline (visit 1) to 22.3 after 1 month (visit 2; p=0.0158) and to 22.2 after 2 months (visit 3; p=0.0223; FIG. 1B). Among the BILAG components reflecting organ system involvement, swollen joint count was reduced after 3-month NAC treatment with dose 3 (XTMIXED z=−2.0; p=0.046) or all doses combined (XTMIXED z=−2.2; p=0.028). The reduction in BILAG was also greater for NAC groups relative to the placebo group as indicated by a significant visit by drug interaction in mixed model analysis (XTMIXED z=−2.62, p=0.009). This analysis also showed a significant reduction of BILAG by NAC dosing group 3 (4.8 g/day; XTMIXED z=−2.19, p=0.029).

In SLE patients treated with all NAC doses combined, FAS was improved from 28.5 at visit 1 to 24.1 at visit 3 (p=0.0006), 23.9 at visit 4 (p=0.005), and 24.8 at visit 5 (p=0.034; FIG. 1C). Mixed model analysis showed a reduction of FAS in NAC dosing group 2 relative to the placebo group (XTMIXED z=−2.08, p=0.038).

### 5. Routine Blood Tests

Routine blood tests included complete blood count, liver and kidney function test, urinalysis and lupus-relevant laboratory tests, such as anti-double-stranded DNA, C3, and C4.

Anti-DNA was reduced in patients exposed to all NAC doses considered together from 78.9±45.2 IU/ml at baseline to 19.5±6.0 IU/ml (p=0.049) after 1 month. C3 and C4 were not affected.

### 6. Compliance:

Compliance of patients was assessed based on self-reporting and pill counts. Pill counts in returned study drug vials indicated a compliance rate of 98.4±1.0%. The principal investigator (PI) did not participate in scoring of patients during enrollment or follow-up visits to avoid bias stemming from potential knowledge of GSH levels due to oversight of immuno-biological and HPLC studies. Upon review of source documents, the PI discovered that two patients received prednisone in excess of 10 mg/day during the study: patient AIM1-005 in the placebo group (for attacks of asthma): 15 mg (visit 2), 20 mg (visit 5); patient AIM 1-007 in the 1st NAC dosing group of 1.2 g/day: 15 mg (visit 1), 13 mg (visit 2), 13 mg (visit 3), 11 mg (visit 4), 11 mg (visit 5). No significant improvement in SLEDAI, BILAG, and FAS was observed upon data analysis leaving out these two patients. However, as tolerance and disease activity, but not prednisone dosage, were clinical outcomes, these two patients, both of whom well tolerated the study drug, were retained for the intent-to-treat analysis.

### Statistical Analysis

Sample size requirements for this study were based on a type I error rate of 0.05, two-tailed testing, and a minimal power level of 0.80, using Sample Power v2 software (SPSS Chicago, Ill.). Estimates of effect size were based on preliminary data (Gergely, P. J. et al., “Mitochondrial hyperpolarization and ATP depletion in patients with systemic lupus erythematosus,” Arth. Rheum., 46: 175-190 (2002)) and the relevant literature to assess/compare mean values of GSH across treatment groups (placebo, lowest NAC, medium NAC, highest NAC dose). This analysis suggested that administration of NAC to a minimum of 8 patients per treatment arm should have 83.7% power to detect a 42% elevation of intracellular GSH in SLE patients (3.60±0.30 ng/g protein) to reach the levels in normal donors (5.11±0.50 ng/g protein).

This study compared the longitudinal effects of three different doses of NAC and a placebo control condition, before (visit 1), during (visit 2, after 1 month; visit 3, after 2 months; visit 4, after 3 months) and following a 3-month intervention (visit 5, after 1 month washout). Thus, this study employed a double-blinded longitudinal trial design comparing 4 groups on observations collected at intervals pre, during and post intervention.

Overall clinical effectiveness of NAC relative to placebo was analyzed with multilevel modeling as implemented in the STATA routine XTMIXED (from StataCorp, College Station, Tex.), with the three nested levels being drug group, subject within drug group and study visit within subject. All models included fixed effects for drug group, study visit and the drug group by study visit interaction along with random intercepts at each design level. Our test for efficacy was the fixed effect for the drug group by study visit interaction which, if significant indicated that the change in outcome scores over time was significantly different about drug groups. The reduction in lupus disease activity SLEDAI scores was greater for the NAC than placebo group, as indicated by a significant visit by drug interaction (z=−2.14, p=0.033). The reduction in BILAG scores was also greater for NAC than placebo group. The overall effect was statistically significant as indicated by a significant visit by drug interaction (z=−2.62, p=0.009). Two tailed paired t-test was used to assess the effects of placebo and of each and all NAC doses on clinical indices and biomarkers recorded on visits 2-5 relative to visit 1; p<0.05 was considered significant. Patients and controls were compared with two-tailed unpaired t-test.

### Immunobiological Outcomes and Assessments

The primary immunobiological outcome was a measurable increase or normalization of GSH previously found to be diminished in PBL by HPLC. (Gergely, P. J. et al., “Mitochondrial hyperpolarization and ATP depletion in patients with systemic lupus erythematosus,” Arth. Rheum., 46: 175-190 (2002)). The secondary immunobiological outcomes were the modulation of ΔΨₘ, ROI production (oxidative stress), activation-induced apoptosis (Gergely, P. J. et al., “Mitochondrial hyperpolarization and ATP depletion in patients with systemic lupus erythematosus,” Arth. Rheum., 46: 175-190 (2002)), activation of mTOR (Fernandez, D. R. et al., “Activation of mTOR controls the loss of TCR in lupus T cells through HRES-1/Rab4-regulated lysosomal degradation, J. Immunol., 182: 2063-2073 (2009)) and expression of FoxP3 (Battaglia, M. et al., “Rapamycin Promotes Expansion of Functional CD4+CD25+FOXP3+Regulatory T Cells of Both Healthy Subjects and Type 1 Diabetic Patients,” J. Immunol., 177(12): 8338-8347 (2006)). The disclosures of each of these references are incorporated by reference herein in their entirety.

HPLC Assay of NAC and GSH:

Reduced glutathione (GSH) was measured by reverse phase ion-exchange HPLC using UV detection at 365 nm, as described in Gergely, P. J. et al., “Mitochondrial hyperpolarization and ATP depletion in patients with systemic lupus erythematosus,” Arth. Rheum., 46: 175-190 (2002); and Hanczko, R. et al., “Prevention of hepatocarcinogenesis and acetaminophen-induced liver failure in transaldolase-deficient mice by N-acetylcysteine,” J. Clin. Invest., 119: 1546-1557 (2009). The disclosures of each of these references are incorporated by reference herein in their entirety.

GSH was measured in whole blood and peripheral blood lymphocytes (PBL) before (visit 1; 0 h) and after the first NAC/placebo dose (visit 1; 3 h and 6 h) and upon each monthly follow-up visit (visits 2-4: between 9-11 am after having taken the last NAC/placebo capsule 8 pm the night before), and after 1 month wash-out (visit 5). HPLC analysis required ˜0.25 ml of whole blood and 5×10⁶ PBL which were obtained from a total of 10 ml of blood collected at each time point.

FIG. 2 shows the effect of NAC on GSH of whole blood (WB) and peripheral blood lymphocytes (PBL) in patients with SLE. FIG. 2A shows HPLC analysis of GSH in whole blood (WB) and peripheral blood lymphocytes (PBL) of untreated SLE patients (n=36) and healthy controls matched for age, gender, and ethnicity (n=42). FIG. 2B shows the effect of NAC and placebo on GSH levels in whole blood of lupus patients. FIG. 2C shows the effect of NAC and placebo on GSH levels in PBL of lupus patients.

At baseline (visit 1, 0 h), GSH was similar in whole blood of lupus and healthy donors. In contrast, GSH was reduced in lupus PBL (FIG. 2A). NAC treatment increased GSH in whole blood of SLE patients after 1 and 2 months (FIG. 2B). In SLE patients, GSH in PBL was increased by a single NAC dose of 1.2 g after 6 h (p=0.022; FIG. 2C) and 2.4 g after 3 h (p=0.032) and 6 h (p=0.003; FIG. 2C). Although GSH levels between the 0 h and 6 h time points in the placebo group were statistically not significant (p=0.053), we appreciated an upward trend that was attributed to the fact that the 0 h sample was obtained after fasting (between 8 am and 9 am) while the 6 h post-NAC sample was obtained after 1 or 2 meals (between 2 pm and 3 pm). These changes were consistent with diurnal variation and peaking of GSH levels in the early afternoon hours due to nutritional factors. (Blanco, R. A. et al., “Diurnal variation in glutathione and cysteine redox states in human plasma,” Am. J. Clin. Nutr., 86: 1016-1023 (2007)). GSH in PBL was increased in SLE patients after 3-month treatment with NAC doses 2 and 3 considered together (visit 4; p=0.027). After one month washout, GSH in PBL dropped below baseline in lupus PBL exposed to NAC dose 3 or doses 2 and 3 combined (FIG. 2C). Placebo did not influence GSH in whole blood or PBL (FIG. 2).

Assessment of Viability, Mitochondrial Transmembrane Potential (ΔΨₘ), Mitochondrial Mass, Ca²⁺ Levels, NO and ROI Production, mTOR Activity, and Foxp3 Expression in Resting and Activated T Cell Subsets and B Cells by Flow Cytometry.

Cell viability was monitored with annexin V conjugated to fluorescein isothiocyanate (annexin V-FITC conjugate), annexin V conjugated to phycoerythrin (annexin V-PE conjugate), or annexin V conjugated to fluorochrome Cy5 (Annexin V-Cy5 conjugate) matched with emission spectra of propidium iodide (PrI) to detect Annexin V+/PrI− apoptotic cells. Mitochondrial transmembrane potential (ΔΨₘ) was monitored with mitochondrial potentiometric dyes such as 3,3′-dihexyloxacarbocyanine iodide ((DiOC₆), using an excitation wavelength of 40 nM and emission at 488 nm, recorded at 525 nm in the FL-1 (green fluorescence) channel); and tetramethylrhodamine, methyl ester ((TMRM), using an excitation wavelength of 100 nM, and emission at 543 nm, recorded at 567 nm in the FL-2 (yellow/orange fluorescence) channel), potential-insensitive mitochondrial dyes such as MitoTracker Green-FM (MTG, 100 nM; excitation: 490 nm, emission: 516 nm recorded in FL-1) or nonyl acridine orange (NAO, 50 nM; excitation: 490 nm, emission: 540 nm recorded in FL-1), superoxide sensing hydroethidine (HE, 1 μM) and H₂O₂-sensing dichlorofluorescein diacetate (DCF-DA, 1 μM), nitric oxide sensor 4-amino-5-methylamino-2′,7′-difluoroflourescein diacetate (DAF-FM, 1 μM, excitation: 495, emission: 515 nm recorded in FL-1), or cytosolic probes (Fluo-3, 1 μM, excitation: 506 nm, emission: 526 nm recorded in FL-1) and mitochondrial Ca²⁺-sensitive fluorescent probes (Rhod-2, 1 μM), respectively.

All metabolic and mitochondrial sensor dyes were obtained from Invitrogen (Carlsbad, Calif.) and used as earlier described in Fernandez, D. R. et al., “Activation of mTOR controls the loss of TCR in lupus T cells through HRES-1/Rab4-regulated lysosomal degradation,” J. Immunol., 182: 2063-2073 (2009); Banki, K. et al., “Glutathione Levels and Sensitivity to Apoptosis Are Regulated by changes in Transaldolase expression,” J. Biol. Chem., 271: 32994-33001 (1996); Banki, K. et al., “Elevation of mitochondrial transmembrane potential and reactive oxygen intermediate levels are early events and occur independently from activation of caspases in Fas signaling,” J. Immunol., 162: 1466-1479 (1999); Nagy, G. et al., “T cell activation-induced mitochondrial hyperpolarization is mediated by Ca²⁺- and redox-dependent production of nitric oxide,” J. Immunol., 171: 5188-5197 (2003); and Perl, A. et al., “Apoptosis and mitochondrial dysfunction in lymphocytes of patients with systemic lupus erythematosus,” In: Perl, A., editor. Autoimmunity: Methods and Protocol. 1 ed. Totowa, N.J.: Humana; 2004 p. 87-114, the entire disclosures of each of which are incorporated by reference herein.

FIG. 3 shows the effect of NAC on Δψₘ (FIG. 3A: DiOC⁶ fluorescence), mitochondrial mass (FIG. 3B: NAO fluorescence), and H₂O₂ levels were measured in T cells rested in culture for 16 h (FIG. 3C: DCF fluorescence). NO production (FIG. 3D: DAF-FM fluorescence), and mitochondrial mass were measured in T cell subsets following CD3/CD28 stimulation for 16 h (FIG. 3E: NAO fluorescence). FIG. 3F: Spontaneous apoptosis rate was enumerated by the percentage of Ann V+/PrI− T cells after culture for 16 h. FIG. 3G: Activation-induced apoptosis was assessed following CD3/CD28 co-stimulation for 16 h. Visits: visit 1, before 1^{st} NAC dose; visit 2, after 1-month treatment; visit 3, after 2-month treatment; visit 4, after 3-month treatment; visit 5, after 1-month washout.

NAC increased Δψₘ (p=0.0001) in all T cells. Mitochondrial hyperpolarization (MHP) was detected in lupus T cells. Interestingly, NAC treatment progressively increased MHP of T cells during treatment, similar to previous findings (Gergely, P. J. et al., “Mitochondrial hyperpolarization and ATP depletion in patients with systemic lupus erythematosus,” Arth. Rheum., 46:175-190 (2002); Nagy, G. et al., “Nitric Oxide-Dependent Mitochondrial Biogenesis Generates Ca²⁺ Signaling Profile of Lupus T Cells,” J. Immunol., 173: 3676-3683 (2004)). (FIG. 3A). Mitochondrial mass (FIG. 3B) and H₂O₂ levels were increased in Double Negative (DN) T cells after 3-month NAC treatment and declined after washout (FIG. 3C). These changes were attributed to enhanced production of NO (1.61±0.17-fold after 3 months, p=0.002; FIG. 3D). Mitochondrial mass was also robustly increased in DN T cells following CD3/CD28 co-stimulation (FIG. 3E)

Spontaneous apoptosis rate of DN T cells was progressively increased in NAC-treated patients from 10.1±1.3% at baseline to 15.2±2.3% after 4 months (p=0.035; FIG. 3F). CD3/CD28-induced apoptosis was increased in NAC-treated patients from 16.0±1.6% at baseline to 25.4±2.6% after 2 months (p=0.0006), 23.5±2.1% after 3 months (p=0.0004), and 22.7±2.6% after 4 months (p=0.0313; FIG. 3G). NAC moderated the expansion of DN T cells from 6.2±0.5% at baseline to 5.3±0.5% after 3 months (p=0.043). Mitochondrial homeostasis, oxidative stress, and apoptosis in T cell subsets of lupus patients were not affected by placebo (data not shown).

Unstimulated cells and cells stimulated with CD3/CD28 for 16 h were examined and cell death pathway selection in T cells was measured by concurrent staining with annexin V-Alexa 647 and propidium iodide (PrI) as well as cell type-specific antigens. T-cell subsets were analyzed by staining with antibodies to CD4, CD8, and CD25. B cells were identified by CD 19 staining

mTOR activity was assessed by phosphorylation of its downstream substrate S6 ribosomal protein (pS6-RP) using a monoclonal antibody to pS6-RP (Cell Signaling; Beverly, Mass.; Cat. No. 4851) in cells permeabilized with Cytofix/CytopermPlus (BD Biosciences).

FIG. 4 shows the detection of increased mTOR activity via phosphorylation of S6 ribosomal protein (pS6-RP) in T-cell subsets from lupus and matched controls. FIG. 4A: Assessment of pS6-RP in CD3⁺, CD4⁺, CD8⁺, and DN T cells from control (blue histograms) and lupus donors (red histograms). Blue/red values show the percentage of cell populations with increased mTOR activity in control and lupus T-cell subsets, respectively. FIG. 4B: Cumulative analysis of mTOR activity in T-cell subsets of all lupus patients relative to all healthy controls. Values represent mean±SEM of cell populations with increased mTOR activity. p values reflect comparison of lupus and healthy donors with unpaired two-tailed t-test before treatment. FIG. 4C: Effect of NAC on mTOR activity measured by the prevalence of pS6-RP^{hi} T cells in lupus patients exposed to all doses considered together. p values reflect comparison to pre-treatment visit 1 using two-tailed paired t-test. FIG. 4D: Effect of NAC on CD3/CD28-induced mTOR activity in T cell subsets of lupus patients exposed to all doses considered together.

Suppression of mTOR by rapamycin has been shown to be associated with improved disease activity in SLE. (Fernandez, D. R. et al., “Activation of mTOR controls the loss of TCRζ [symbol] adation,” J. Immunol., 182: 2063-2073 (2009); Fernandez, D. et al., “Rapamycin reduces disease activity and normalizes T-cell activation-induced calcium fluxing in patients with systemic lupus erythematosus,” Arth. Rheum., 54: 2983-2988 (2006)). This Example shows increased mTOR activity in SLE as evidenced by 2.2±0.45-fold greater prevalence of pS6-RP^{hi} T cells in SLE patients (p=0.007). The absolute frequency of pS6-RP^{hi} T cells was greatest in the DN compartment. (FIGS. 4A and 4B). NAC depleted pS6-RP^{hi} T cells in the DN T-cell compartment from 22.5±3.7% at baseline to 16.9±2.5% after 2 months (p=0.0104), 16.4±2.4% after 3 months (p=0.0095), and 12.1±2.4% after 4 months (p=0.0009; FIG. 4C). NAC diminished CD3/CD28-induced mTOR activation in all T cells after 2 months and 3 months, which rebounded after washout (FIG. 4D). mTOR activity was not affected by placebo (data not shown). CD3/CD28-stimulated mTOR activity declined in DN T cells at visit 2 in the placebo group (data not shown), however, this effect did not show a sustained or progressive time course and, therefore, it was not considered biologically significant.

Foxp3 expression was measured in permeabilized cells using Alexa-647-conjugated antibody from BioLegend (San Diego, Calif.; cat No 320014). Up to 11 parameters were recorded simultaneously using a Becton Dickinson LSRII flow cytometer equipped with 20 mW solid-state Nd-YAG (emission at 355 nm), 20 mW argon (emission at 488 nm), 10 mW diode pumped solid state yellow-green (emission 561 nm) and 16 mW heliumneon lasers (emission at 634 nm). Each patient's cells were processed and analyzed in parallel with a matched control.

FIG. 5 shows the simulation of FoxP3 expression by NAC in lupus T cells. FIG. 5A: FoxP3 expression in CD4⁺/CD25⁺ and CD8⁺/CD25⁺ T cell subsets of lupus and control donors matched for age, gender, and ethnicity by flow cytometry. Red and blue values indicate percentage of FoxP3⁺ cells in lupus and control donors, respectively. FIG. 5B: Cumulative analysis of FoxP3 expression in CD25⁺ T-cell subsets in lupus subjects and matched controls. p values reflect comparison with two-tailed unpaired t-test. FIG. 5C: Effect of NAC on Foxp3 expression in CD25⁺ T cell subsets of lupus patients exposed to all doses considered together. p values reflect comparison with two-tailed paired t-test.

Before treatment, FoxP3⁺ cells were reduced within the CD25⁺ T-cell compartment of SLE patients relative to healthy controls (p=6.0×10⁻⁵; FIGS. 5A and B). FoxP3⁺ cells within the CD4⁺/CD25⁺ T-cell compartment were reduced at 37.8±2.4% in SLE patients relative to 47.2±2.3% in controls (p=9.1×10⁻⁵; FIG. 5B). FoxP3⁺ cells were also reduced within the CD8⁺/CD25⁺ T-cell compartment at 10.7±2.0% in SLE patients relative to 26.7±4.4% in controls (p=0.002; FIG. 5B). Since rapamycin expanded CD4⁺/CD25⁺/FoxP3⁺ T cells in patients with SLE, it was important to determine whether mTOR blockade by NAC affected FoxP3 expression. (Lai, Z. et al., “Reversal of CD3/CD4/CD25/Foxp3 Treg Depletion in Active SLE Patients with Rapamycin,” Arthritis & Rheumatism, 62 (Suppl. 10): 1185-DOI: 10.1002/art.28951. 2010). This Example shows that the percentage of FoxP3+ cells within the CD4⁺/CD25⁺ T-cell compartment was increased in all NAC-treated patients (p=0.045; FIG. 5C). FoxP3 expression was not affected in patients exposed to placebo (data not shown). CD4⁺/CD25⁺/FoxP3⁺ T cells were expanded in patients exposed to 4.8 g/day NAC from 3.2±0.6% at baseline to 5.2±0.9% after 2 months (p=0.018). FoxP3 expression was also induced in CD25⁺ DN T cells from 1.29±0.23% at baseline to 2.24±0.38% after 2-month NAC treatment (p=0.0160).

In summary, NAC increased GSH in PBL and, improved disease activity in SLE patients through the disruption of the MHP-mTOR pathway in T cells. Considered together, 2.4 g and 4.8 g NAC reduced: 1) SLEDAI after 1 month (p=0.0007), 2 months (p=0.0009), 3 months (p=0.0030) and 4 months (p=0.0046); 2) BILAG after 1 month (p=0.029) and 3 months (p=0.0009); and 3) FAS after 2 months (p=0.002) and 3 months (p=0.004). NAC increased Δψₘ (p=0.0001) in all T cells, it profoundly reduced mTOR activity (p=0.0001), enhanced apoptosis (p=0.0004) and reversed expansion of CD4⁻/CD8⁻ T cells (1.35V0.12-fold; p=0.008), stimulated Foxp3 expression in CD4⁺/CD25⁺ T cells (p=0.045), and reduced anti-DNA production (p=0.049).

The low GSH in PBL, but not in whole blood, suggests that the metabolic dysfunction in lupus is confined to the immune system. (Fernandez, D. and Perl, A. “Metabolic control of T cell activation and death in SLE,” Autoimmun. Rev., 8: 184-189 (2009)). FIG. 6 shows a schematic functional hierarchy of metabolic biomarkers of T-cell dysfunction in patients with SLE, depicting the proposed site of impact by NAC. MHP is caused by exposure to nitric oxide (NO). De novo synthesis of NO and maintenance of GSH in reduced form are both dependent on the production of NADPH by the pentose phosphate pathway (PPP). MHP causes mTOR activation which in turn controls the expression of the transcription factor FoxP3. This Example shows that NAC increased GSH in PBL and, improved disease activity in SLE patients through the disruption of the MHP-mTOR pathway in T cells (FIG. 6).

Δψₘ is subject to regulation by an oxidation-reduction equilibrium of ROI, pyridine nucleotides (NADH/NAD+NADPH/NADP) and GSH. (Fernandez, D. and Perl, A. “Metabolic control of T cell activation and death in SLE,” Autoimmun. Rev., 8: 184-189 (2009)). Without being bound by theory, it is believed that NAC can modulate mitochondrial hyperpolarization (MHP), directly, via neutralizing ROI or, indirectly, via sparing NADPH and promoting de novo GSH production. This Example shows that Δψₘ and mitochondrial mass were increased in T cells by NAC, particularly in the DN compartment, with reversal of these changes after washout. NAC-induced MHP occurred with a marked increase in NO production, which is required for mitochondrial biogenesis. (Nagy, G. et al., “Nitric Oxide-Dependent Mitochondrial Biogenesis Generates Ca²⁺ Signaling Profile of Lupus T Cells,” J. Immunol., 173: 3676-3683 (2004)). In turn, NO production depends on the availability of NADPH. Without being bound by theory, it is believed that increased NO production can result from sparing of NADPH by NAC. (Fernandez, D. and Perl, A. “Metabolic control of T cell activation and death in SLE,” Autoimmun. Rev., 8: 184-189 (2009)). Without being bound by theory, NAC acts by blocking mTOR, which is a sensor of Δψₘ and oxidative stress in lupus T cells. (Fernandez, D. R. et al., “Activation of mTOR controls the loss of TCRζ □ in lupus T cells through HRES-1/Rab4-regulated lysosomal degradation,” J. Immunol., 182: 2063-2073 (2009)). In addition, suppression of mTOR by NAC was accompanied by increased FoxP3 expression in CD4⁺/CD25⁺ T cells. These results suggest that the effect of NAC on the immune system is cell type-specific and it occurs by disconnecting the activation of mTOR from the elevation of Δψₘ in lupus T cells. Such direct inhibitory effect of NAC was confirmed by blocking of CD3/CD28 stimulation-induced mTOR activity in normal PBL upon pretreatment by NAC in vitro (data not shown).

### Example 2

### Attention Deficit and Hyperactivity Disorder Scores in SLE and Effect of NAC on the Same

### Human Subjects

The validated ADHD Self-Report Scale (ASRS) Symptom Checklist (Table 5) (Kessler, R. C. et al., “The World Health Organization Adult ADHD Self-Report Scale (ASRS): a short screening scale for use in the general population,” Psychol. Med., 35(2): 245-256 (2005)) was used to assess 49 SLE patients. A first cohort of 24 patients was enrolled in a treatment trial with NAC (IND No: 101,320; clinicaltrials.gov identifier: NCT00775476). A second cohort of 25 patients was not enrolled in this trial. As controls, healthy donors matched for ethnicity, gender, and age of the SLE patient within 10 years, were also asked to complete the ASRS checklist in parallel when donating blood for immunobiological studies. (See Example 1). In the SLE group, 44 of 49 patients were Caucasian, four was African-American, and one was Asian. In the control group, 45 of the 46 donors were Caucasian and one was African-American. Mean age was 45.9 years (standard deviation, SEM=1.8, range=20-67) in the SLE group. Mean age was 48.0 years (SEM=1.5, range=26-64) in the control group. Three SLE patients and one healthy control were Caucasian males, all other subjects were females.

### Clinical Study Design

The clinical trial design, eligibility criteria, randomization, blinding, monitoring of safety, tolerance and efficacy on NAC in patients with SLE was as described in Example 1, approved by the Food and Drug Administration (IND No: 101,320; clinicaltrials.gov identifier: NCT00775476). As an addendum to the Example 1 study, ASRS was evaluated as an exploratory outcome in 24 SLE patients randomized to receive orally either placebo or NAC in one of two treatment arms: 1,200 mg or 2,400 mg twice daily for three months. 12 patients were enrolled per dosing group, 9 received NAC while 3 received placebo. Due to missing data, 6 patients enrolled in the placebo arm and 8 patients enrolled into each of the two NAC treatment arms could be evaluated.

The ASRS is an 18-item scale that is used to assess the current status of the 18 DSM-IV symptoms of ADHD in adults. (Kessler, R. C. et al., “The World Health Organization Adult ADHD Self-Report Scale (ASRS): a short screening scale for use in the general population,” Psychol. Med., 35(2): 245-256 (2005)). Symptoms are rated on a frequency basis: 0=never, 1=rarely, 2=sometimes, 3=often, and 4=very often. Nine items assess inattention and nine assess hyperactivity-impulsivity. The 9 inattentive symptoms are summed to create the ASRS A subscale; the 9 hyperactive-impulsive symptoms are summed to compute the ASRS B subscale. These two scales are summed to compute the total score. For all scales, higher scores indicate more symptoms. The scale has high concurrent validity with a rater-administered ADHD symptom scale. (Adler, L. A. et al., “Validity of Pilot Adult ADHD Self-Report Scale (ASRS) to Rate Adult ADHD Symptoms,” Ann Clin. Psychiatry 18(3): 145-148 (2006)). As described in Example 1, SLE disease activity was assessed by using the British Isles Lupus Assessment Group (BILAG) (Isenberg, D. A. et al., “BILAG 2004. Development and initial validation of an updated version of the British Isles Lupus Assessment Group's disease activity index for patients with systemic lupus erythematosus,” Rheumatology, 44(7): 902-906 (2005)) and Systemic Lupus Erythematosus Disease Activity Index (SLEDAI) (Hochberg, M. C., “Updating the American College of Rheumatology revised criteria for the classification of systemic lupus erythematosus,” Arth. Rheum., 40(9): 1725 (1997)). Fatigue was estimated using the Fatigue Assessment Scale (FAS) (Michielsen, H. J. et al., “Psychometric qualities of a brief self-rated fatigue measure: The Fatigue Assessment Scale,” J. Psychosom. Res., 54(4): 345-352 (2003)).

### Statistical Analyses

Overall clinical effectiveness of NAC relative to placebo was analyzed with multilevel modeling as implemented in the STATA routine XTMIXED (from StataCorp, College Station, Tex.), with the three nested levels being drug group, subject within drug group and study visit within subject. All models included fixed effects for drug group, study visit and the drug group by study visit interaction along with random intercepts at each design level. Our test for efficacy was the fixed effect for the drug group by study visit interaction which, if significant indicated that the change in outcome scores over time was significantly different about drug groups. Two-tailed paired t-test was used to assess the effects of placebo and of each and all NAC doses on clinical indices recorded on visits 2-5 relative to visit 1; p<0.05 was considered significant. Patients and controls were compared with two-tailed unpaired t-test. Correlations and t-test were performed with Prism (GraphPad, San Diego, Calif.).

### Results

The ADHD Scores are Elevated and Correlate with Disease Activity in Patients with SLE.

FIG. 7 shows ASRS A (cognitive/inattentive), ASRS B (hyperactivity/impulsive), and total ASRS scores (ASRS Total) in patients with SLE and healthy controls matched for age within 10 years, gender, and ethnicity. Left panel, Analysis of cohort I comprising 24 SLE patients and 22 healthy subjects enrolled in a treatment trial of NAC (IND No: 101,320; clinicaltrials.gov identifier: NCT00775476). Middle panel: Analysis of cohort II comprising 25 SLE patients and 24 healthy subjects. Right panel, Analysis of cohorts I and II are combined. FIG. 8 shows the correlation of ASRS A and ASRS B scores with SLEDAI, BILAG, and FAS in 49 patients with SLE. Pearson's r values are shown for correlations with p<0.05.

The mean±SEM of cognitive/inattentive components of ASRS (ASRS A scores), hyperactivity/impulsive components of ASRS (ASRS B scores), and total ASRS scores (ASRS Total) were 10.41±1.02, 9.61±1.21 and 20.02±1.98, respectively, in the control population of 46 healthy subjects. As shown in FIG. 7, the mean±SEM of ASRS A, ASRS B and ASRS total scores were increased at 17.23±1.55 (p=0.0001), 14.36±1.32 (p=0.004) and 31.59±2.75 in the first SLE group of 24 patients enrolled in the NAC trial (see below) (p=0.0004). The mean±SEM of ASRS A, ASRS B and ASRS total scores were also increased at 18.24±0.90 (p=0.00003), 14.0±1.02 (p=0.034) and 32.35±1.62 in a second cohort of 25 patients outside the NAC trial (p=0.0004). In all 49 SLE patients combined, the mean±SEM of ASRS A, ASRS B and ASRS total scores were also increased at 17.37±1.03 (p=3×10⁻⁷), 14.51±0.89 (p=2×10⁻⁴) and 31.92±1.74 (p=8×10⁻⁷).

Using Pearson's correlation, fatigue (FAS) scores of SLE patients correlated with ASRS A (r=0.73, p<0.0001), ASRS B (r=0.47, p=0.0006) and ASRS total scores (r=0.67, p<0.0001; FIG. 8). SLEDAI correlated with ASRS A (r=0.53; p<0.0001) and ASRS total scores (r=0.45, p=0.0009; FIG. 8). BILAG also correlated with ASRS A (r=0.36; p=0.011) and ASRS total scores (r=0.31, p=0.025; FIG. 8). There were also significant correlations between SLEDAI and BILAG (r=0.51, p=0.0002), BILAG and FAS (r=0.40, p=0.0043), and SLEDAI and FAS (r=0.24, p=0.042; data not shown). 26/49 patients had fibromyalgia (FM) and exhibited elevated FAS scores (31.5) relative to 23 patients without FM (26.6; p=0.27). 70.8% of patients with FM and 30.2% of SLE patients without FM were unemployed (p=0.007). 23/26 (88.5%) of SLE patients with FM and none of the patients without FM had major neuropsychiatric manifestations, such as depression, stroke, or history of seizures (p=3.2×10⁻¹⁸). 24/49 patients were unemployed and exhibited increased prevalence of FM (68%) and fatigue scores (FAS: 31.4) relative to 22 patients having jobs (FM: 32%, p=0.007; FAS: 26.9, p=0.022). ASRS A, ASRS B, ASRS total scores, SLEDAI, or BILAG was not elevated in SLE patients with FM, depression, antidepressant use or lack of employment relative patients without these conditions (data not shown).

The elevated ASRS scores indicate the presence of clinically significant symptoms of ADHD in patients with SLE relative to healthy controls matched for age, gender, and ethnic background. Elevated ASRS scores were noted in two independent SLE cohorts, evaluated both separately and together. ASRS scores correlated with SLEDAI, BILAG and FAS scores but not with FM, employment, or existing diagnosis of depression. FAS scores positively correlated with FM, unemployment, and depression. Without being bound by theory, ADHD symptoms can be a source of cognitive impairment in SLE, which could lead to functional disability.

NAC Treatment Reduces ADHD Scores in Patients with SLE.

As described in Example 1, NAC significantly improved lupus disease activity, as measured by SLEDAI, BILAG, and FAS scores in a double-blind placebo-controlled randomized pilot study of 36 SLE patients. Example 2 shows the effect of 2.4 g/day NAC (Dose 2) and 4.8 g/day NAC (Dose 3) relative to placebo on ASRS scores of SLE patients. FIG. 9 shows the effect of NAC and placebo on ASRS scores (ASRS total, left panel; ASRS A inattentive components, right panel) in 24 SLE patients exposed to placebo (n=6), 2.4 g/day NAC (NAC Dose 2; n=9), 4.8 g/day NAC (NAC Dose 3; n=9), or NAC Doses 2 and 3 considered together (NAC All doses; n=18). Data represent mean±SEM. p values reflect comparison of pretreatment values (visit 1) to values after treatment for 1 month (visit 2), 2 months (visit 3), 3 months (visit 4), or 4 months (visit 5, 3 months of treatment followed by 1 month washout) using two-tailed paired t-test.

ASRS A, ASRS B and ASRS Total scores were similar at visit 1 between the placebo and NAC dosing groups. Patients exposed to NAC Dose 3 had diminished ASRS Total scores after 2 and 3 months of treatment, while ASRS Total scores were reduced in patients treated with NAC Doses 2 and 3 combined after 3 months (FIG. 9). Using multilevel modeling, the reduction in ASRS Total scores was greater in the NAC (Doses 2 and 3 combined) than placebo group as indicated by a significant visit by drug interaction (XTMIXED z=−2.09, p=0.037). The cognitive/inattentive components ASRS, ASRS A scores, were influenced by NAC. Dose 2 (z=−3.31, p=0.001) and Dose 3 of NAC (z=−4.04, p<0.0001) as well as NAC Doses 2 and 3 combined reduced the cognitive/inattentive ASRS A scores (z=−3.41, p=0.001). The hyperactivity/impulsive components of ASRS scores (ASRS B scores) were not affected by NAC Dose 2 and 3, alone or combined (data not shown). At the end of 3-month treatment, the standardized mean difference effect size for NAC Dose 3 (Cohen's D) was 0.72 for ASRS Total scores, 0.71 for ASRS A scores and 0.44 for ASRS B scores.

The results suggest that ASRS may be a useful instrument to detect cognitive dysfunction, an important neuropsychiatric manifestation in SLE. (Liang, M. H. et al., “The American College of Rheumatology nomenclature and case definitions for neuropsychiatric lupus syndromes,” Arth. Rheum., 42(4): 599-608 (1999)). Longitudinal studies indicate that ADHD symptoms predict the subsequent onset of severe neuropsychiatric disorders that frequently follow the onset of idiopathic ADHD in children. (Biederman, J. et al., “Adult psychiatric outcomes of girls with attention deficit hyperactivity disorder: 11-year follow-up in a longitudinal case-control study.,” Am. J. Psychiatry, 167(4): 409-417 (2010)).

### EQUIVALENTS

The present invention may be embodied in other specific forms without departing from the spirit or essential attributes thereof and, accordingly, reference should be made to the appended claims, rather than to the foregoing specification, as indicating the scope of the invention.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed:

1. A method of treating a lupus condition in a subject in need thereof, comprising:
(a) providing a pharmaceutical composition comprising a therapeutic amount of a compound N-acetyl-L-cysteine (NAC) of Formula I: [chemical structure]
or a pharmaceutically acceptable salt, solvate, prodrug, or a derivative thereof; and a pharmaceutically acceptable carrier; and
(b) administering the pharmaceutical composition to the subject, wherein the therapeutic amount is effective to decrease activity of mammalian target of rapamycin (mTOR) and to treat one or more symptoms of the lupus condition.

2. The method according to claim 1, wherein the lupus condition is systemic lupus erythematosus (SLE).

3. The method according to claim 1, wherein the therapeutic amount of N-acetyl-L-cysteine (NAC) for an adult is a maximum daily dose of about 4800 mg/day to about 8000 mg/day.

4. The method according to claim 1, wherein the therapeutic amount of the N-acetyl-L-cysteine (NAC) is effective to reduce lupus disease activity in the subject compared to an untreated control.

5. The method according to claim 4, wherein the lupus disease activity is measured by a disease activity score selected from the group consisting of systemic lupus erythematosus disease activity index (SLEDAI) score, British Isles Lupus Assessment Group (BILAG) score, fatigue assessment scale (FAS) score, or a combination thereof.

6. The method according to claim 5, wherein the systemic lupus erythematosus disease activity index (SLEDAI) score of the subject is reduced compared to an untreated control at least after at least 7 days of the administration, after at least 14 days of the administration, after at least 1 month of the administration, after at least 2 months of the administration, after at least 3 months of the administration, or after at least 4 months of the administration.

7. The method according to claim 6, wherein the systemic lupus erythematosus disease activity index (SLEDAI) score of the subject is reduced by at least 1 point to at least 2.3 points compared to an untreated control after at least 1 month of the administration.

8. The method according to claim 5, wherein the British Isles Lupus Assessment Group (BILAG) score of the subject is reduced compared to an untreated control at least after at least 7 days of the administration, after at least 14 days of the administration, after at least 1 month of the administration, after at least 2 months of the administration, after at least 3 months of the administration, or after at least 4 months of the administration.

9. The method according to claim 8, wherein the British Isles Lupus Assessment Group (BILAG) score of the subject is reduced by at least 1.0 point to at least 5.0 points compared to an untreated control after at least 1 month of the administration.

10. The method according to claim 5, wherein the fatigue assessment scale (FAS) score of the subject is reduced compared to an untreated control at least after at least 7 days of the administration, after at least 14 days of the administration, after at least 1 month of the administration, after at least 2 months of the administration, after at least 3 months of the administration, or after at least 4 months of the administration.

11. The method according to claim 10, wherein the fatigue assessment scale (FAS) score of the subject is reduced by at least 1.0 point to at least 5.0 points compared to an untreated control after at least 1 month of the administration.

12. The method according to claim 1, wherein the therapeutic amount of the N-acetyl-L-cysteine (NAC) is effective to increase activation-induced apoptotic rate of double negative (DN) T cells of the subject compared to an untreated control after at least 7 days of the administration, after at least 14 days of the administration, after at least 1 month of the administration, after at least 2 months of the administration, after at least 3 months of the administration, or after at least 4 months of the administration.

13. The method according to claim 1, wherein the therapeutic amount of the N-acetyl-L-cysteine (NAC) is effective to decrease activity of mammalian target of rapamycin (mTOR) compared to an untreated control after at least 7 days of the administration, after at least 14 days of the administration, after at least 1 month of the administration, after at least 2 months of the administration, after at least 3 months of the administration, or after at least 4 months of the administration.

14. The method according to claim 1, wherein the therapeutic amount of the N-acetyl-L-cysteine (NAC) is effective to increase number of FoxP3+CD8+CD25+ T cells compared to an untreated control after at least 7 days of the administration, after at least 14 days of the administration, after at least 1 month of the administration, after at least 2 months of the administration, after at least 3 months of the administration, or after at least 4 months of the administration.

15. The method according to claim 1, wherein the therapeutic amount of the N-acetyl-L-cysteine (NAC) is effective to reduce a cognitive/inattentive component of attention deficit and hyperactivity (ADHD) self-report scale (ASRS) compared to an untreated control after at least 7 days of the administration, after at least 14 days of the administration, after at least 1 month of the administration, after at least 2 months of the administration, after at least 3 months of the administration, or after at least 4 months of the administration.

16. The method according to claim 1, wherein the pharmaceutical composition further comprises at least one additional therapeutic agent selected from the group consisting of a non-steroidal anti-inflammatory agent, an antimalarial agent, a corticosteroid, a cytotoxic agent, an immunosuppressive agent, a biologic, or a combination thereof.

17. The method according to claim 16, wherein the non-steroidal anti-inflammatory agent is selected from the group consisting of aspirin, arthopan, celecoxib, diclofenac, etodolac, fenprofen, flurbiprofen, ibuprofen, ketoprofen, meclofamate, meloxicam, nabumetone, naproxen, oxaprozin, piroxicam, rofecoxib, sulindac, tolmetin, acetaminophen, or a combination thereof.

18. The method according to claim 16, wherein the antimalarial agent is selected from the group consisting of hydroxycloroquine, chloroquine, quinicrine, or a combination thereof.

19. The method according to claim 16, wherein the corticosteroid is in the form of a topical cream or ointment, a tablet, or an intravenous formulation.

20. The method according to claim 19, wherein the topical cream is selected from the group consisting of clobetasol, halobetasol, hydrocortisone, triamcinolone, betamethasone, fluocinolone, fluocinonide, or a combination thereof.

21. The method according to claim 19, wherein the tablet is selected from the group consisting of prednisone, prednisolone, ethylprednisone, or a combination thereof.

22. The method according to claim 19, wherein the intravenous formulation is selected from the group consisting of methylprednisone, hydrocortisone, or a combination thereof.

23. The method according to claim 16, wherein the cytotoxic agent is selected from the group consisting of azathioprine, cyclophosphamide, mycophenolate mofetil, cyclosporine A, methotrexate, chlorambucil, or a combination thereof.

24. The method according to claim 16, wherein the immunosuppressive agent is selected from the group consisting of azathioprine, cyclophosphamide, mycophenolate mofetil, cyclosporine A, methotrexate, chlorambucil, or a combination thereof.

25. The method according to claim 16, wherein the biologic is selected from the group consisting of a B-cell target biologic, a T cell target biologic, a spleen tyrosine kinase antagonist, a tumor necrosis factor (TNF) antagonist, an interferon antagonist, an interleukin-6-receptor antagonist, or a combination thereof.

26. The method according to claim 1, wherein the administering in step (b) is orally, topically, parenterally, buccally, sublingually, by inhalation, or rectally.

27. The method according to claim 26, wherein the administering in step (b) is orally.

28. The method according to claim 27, wherein the pharmaceutical composition is in form of a tablet, a pill, a gel, a troche, a lozenge, an aqueous suspension, an oily suspension, a capsule, or a syrup.

29. The method according to claim 26, wherein the administering in step (b) is topically.

30. The method according to claim 29, wherein the pharmaceutical composition is in the form of an aqueous suspension, an oily suspension, an emulsion, a cream, or a patch.

31. The method according to claim 26, wherein the administering in step (b) is parenterally.

32. The method according to claim 31, wherein the pharmaceutical composition is in the form of an injectable solution, a gel, an aqueous suspension, an oily suspension, a granule, a bead, an emulsion, or an implant.

33. The method according to claim 26, wherein the administering in step (b) is buccally.

34. The method according to claim 33, wherein the pharmaceutical composition is in the form of a tablet, a pill, a gel, a troche, a lozenge, an aqueous suspension, an oily suspension, a capsule, or a syrup.

35. The method according to claim 26, wherein the administering in step (b) is sublingually.

36. The method according to claim 35, wherein the pharmaceutical composition is in the form of a tablet, a pill, a gel, a troche, a lozenge, an aqueous suspension, an oily suspension, a capsule, or a syrup.

37. The method according to claim 26, wherein the administering in step (b) is rectally.

38. The method according to claim 37, wherein the pharmaceutical composition is in the form of a suppository or an insert.

39. A kit for treating a lupus condition in a subject in need thereof, comprising:
(a) a first packaging material containing a pharmaceutical composition comprising a therapeutic amount of a compound N-acetyl-L-cysteine (NAC) of Formula I: [chemical structure]
or a pharmaceutically acceptable salt, solvate, prodrug, or a derivative thereof; and a pharmaceutically acceptable carrier; and
(b) a means for administering the composition.

40. The kit according to claim 39, wherein the lupus condition is systemic lupus erythematosus (SLE).

41. The kit according to claim 39, wherein the therapeutic amount of N-acetyl-L-cysteine (NAC) in the kit for an adult is a maximum daily dose of about 4800 mg/day to about 8000 mg/day.

42. The kit according to claim 39, wherein the therapeutic amount of the N-acetyl-L-cysteine (NAC) is effective to reduce lupus disease activity in the subject compared to an untreated control.

43. The kit according to claim 42, wherein the lupus disease activity is measured by a disease activity score selected from the group consisting of systemic lupus erythematosus disease activity index (SLEDAI) score, British Isles Lupus Assessment Group (BILAG) score, fatigue assessment scale (FAS) score, or a combination thereof.

44. The kit according to claim 43, wherein the systemic lupus erythematosus disease activity index (SLEDAI) score of the subject is reduced compared to an untreated control at least after at least 7 days of the administration, after at least 14 days of the administration, after at least 1 month of the administration, after at least 2 months of the administration, after at least 3 months of the administration, or after at least 4 months of the administration.

45. The kit according to claim 44, wherein the systemic lupus erythematosus disease activity index (SLEDAI) score of the subject is reduced by at least 1 point to at least 2.3 points compared to an untreated control after at least 1 month of the administration.

46. The kit according to claim 43, wherein the British Isles Lupus Assessment Group (BILAG) score of the subject is reduced compared to an untreated control at least after at least 7 days of the administration, after at least 14 days of the administration, after at least 1 month of the administration, after at least 2 months of the administration, after at least 3 months of the administration, or after at least 4 months of the administration.

47. The kit according to claim 46, wherein the British Isles Lupus Assessment Group (BILAG) score of the subject is reduced by at least 1.0 point to at least 5.0 points compared to an untreated control after at least 1 month of the administration.

48. The kit according to claim 43, wherein the fatigue assessment scale (FAS) score of the subject is reduced compared to an untreated control at least after at least 7 days of the administration, after at least 14 days of the administration, after at least 1 month of the administration, after at least 2 months of the administration, after at least 3 months of the administration, or after at least 4 months of the administration.

49. The kit according to claim 48, wherein the fatigue assessment scale (FAS) score of the subject is reduced by at least 1.0 point to at least 5.0 points compared to an untreated control after at least 1 month of the administration.

50. The kit according to claim 39, wherein the therapeutic amount of the N-acetyl-L-cysteine (NAC) is effective to increase activation-induced apoptotic rate of double negative (DN) T cells of the subject compared to an untreated control after at least 7 days of the administration, after at least 14 days of the administration, after at least 1 month of the administration, after at least 2 months of the administration, after at least 3 months of the administration, or after at least 4 months of the administration.

51. The kit according to claim 39, wherein the therapeutic amount of the N-acetyl-L-cysteine (NAC) is effective to decrease activity of mammalian target of rapamycin (mTOR) compared to an untreated control after at least 7 days of the administration, after at least 14 days of the administration, after at least 1 month of the administration, after at least 2 months of the administration, after at least 3 months of the administration, or after at least 4 months of the administration.

52. The kit according to claim 39, wherein the therapeutic amount of the N-acetyl-L-cysteine (NAC) is effective to increase number of FoxP3+CD8+CD25+ T cells compared to an untreated control after at least 7 days of the administration, after at least 14 days of the administration, after at least 1 month of the administration, after at least 2 months of the administration, after at least 3 months of the administration, or after at least 4 months of the administration.

53. The kit according to claim 39, wherein the therapeutic amount of the N-acetyl-L-cysteine (NAC) is effective to reduce cognitive/inattentive component of attention deficit and hyperactivity (ADHD) self-report scale (ASRS) compared to an untreated control after at least 7 days of the administration, after at least 14 days of the administration, after at least 1 month of the administration, after at least 2 months of the administration, after at least 3 months of the administration, or after at least 4 months of the administration.

54. The kit according to claim 39, wherein the pharmaceutical composition further a second packaging material containing at least one additional therapeutic agent selected from the group consisting of a non-steroidal anti-inflammatory agent, an antimalarial agent, a corticosteroid, a cytotoxic agent, an immunosuppressive agent, a biologic, or a combination thereof.

55. The kit according to claim 54, wherein the non-steroidal anti-inflammatory agent is selected from the group consisting of aspirin, arthopan, celecoxib, diclofenac, etodolac, fenprofen, flurbiprofen, ibuprofen, ketoprofen, meclofamate, meloxicam, nabumetone, naproxen, oxaprozin, piroxicam, rofecoxib, sulindac, tolmetin, acetaminophen, or a combination thereof.

56. The kit according to claim 54, wherein the antimalarial agent is selected from the group consisting of hydroxycloroquine, chloroquine, quinicrine, or a combination thereof.

57. The kit according to claim 54, wherein the corticosteroid is in the form of a topical cream or ointment, a tablet, or an intravenous formulation.

58. The kit according to claim 57, the topical cream is selected from the group consisting of clobetasol, halobetasol, hydrocortisone, triamcinolone, betamethasone, fluocinolone, fluocinonide, or a combination thereof.

59. The kit according to claim 57, the tablet is selected from the group consisting of prednisone, prednisolone, ethylprednisone, or a combination thereof.

60. The kit according to claim 57, the intravenous formulation is selected from the group consisting of methylprednisone, hydrocortisone, or a combination thereof.

61. The kit according to claim 54, wherein the cytotoxic agent is selected from the group consisting of azathioprine, cyclophosphamide, mycophenolate mofetil, cyclosporine A, methotrexate, chlorambucil, or a combination thereof.

62. The kit according to claim 54, wherein the immunosuppressive agent is selected from the group consisting of azathioprine, cyclophosphamide, mycophenolate mofetil, cyclosporine A, methotrexate, chlorambucil, or a combination thereof.

63. The kit according to claim 54, wherein the biologic is selected from the group consisting of a B-cell target biologic, a T cell target biologic, a spleen tyrosine kinase antagonist, a tumor necrosis factor (TNF) antagonist, an interferon antagonist, an interleukin-6-receptor antagonist, or a combination thereof.

64. The kit according to claim 39, wherein the means (b) for administering the composition is a syringe, a nebulizer, an inhaler, a dropper, a tablet, a pill, a gel, a troche, a lozenge, an aqueous suspension, an oily suspension, a capsule, a syrup, an emulsion, a cream, a patch, an injectable solution, a granule, a bead, an implant, a suppository, an insert, or a combination thereof.

65. The kit according to claim 39, wherein the kit further comprises instructions for use.

66. The kit according to claim 39, wherein at least one of the first or second packaging material is selected from the group consisting of a box, a pouch, a vial, a bottle, a tube, a blister pack, or a combination thereof.
